(19) United States
(12) Patent Application Publication
(10) Pub. No.: US 2010/0047261 A1
(43) Pub. Date: Feb. 25, 2010

(54) BASE-MODIFIED RNA FOR INCREASING THE EXPRESSION OF A PROTEIN

(75) Inventors: Ingmar Hoerr, Tubingen (DE); Florian Von Der Mulbe, Tubingen (DE)

Correspondence Address: CONNOLLY BOVE LODGE & HUTZ, LLP, P O BOX 2207, WILMINGTON, DE, 19899, US

(73) Assignee: CureVac GmbH, Tubingen (DE)

(21) Appl. No.: 12/446,912

(22) PCT Filed: Oct. 31, 2007
(86) PCT No.: PCT/EP07/09469

§ 371 Date: Oct. 27, 2009

(30) Foreign Application Priority Data
Oct. 31, 2006 (DE) 102006051516.1-41

(51) Int. Cl. A61K 39/00; C12P 19/34; A61K 35/12; C40B 40/08; A61P 35/00; A61P 31/00

(52) U.S. Cl. 424/184.1; 435/91.2; 424/93.7; 506/17; 536/23.1

(57) ABSTRACT

The present application describes a base-modified RNA and the use thereof for increasing the expression of a protein and for the preparation of a pharmaceutical composition, especially a vaccine, for the treatment of tumours and cancer diseases, heart and circulatory diseases, infectious diseases, autoimmune diseases or monogenetic diseases, for example in gene therapy. The present invention further describes an in vitro transcription method, in vitro methods for increasing the expression of a protein using the base-modified RNA, and an in vivo method.

The present application describes a base-modified RNA and the use thereof for increasing the expression of a protein and for the preparation of a pharmaceutical composition, especially a vaccine, for the treatment of tumours and cancer diseases, heart and circulatory diseases, infectious diseases, autoimmune diseases or monogenetic diseases, for example in gene therapy. The present invention further describes an in vitro transcription method, in vitro methods for increasing the expression of a protein using the base-modified RNA, and an ex vivo and in vivo method.

Apart from heart and circulatory diseases and infectious diseases, the occurrence of tumours and cancer diseases is one of the most frequent causes of death in modern society and in most cases is associated with considerable costs in terms of therapy and subsequent rehabilitation measures. The treatment of tumours and cancer diseases is greatly dependent, for example, on the type of tumour that occurs and is nowadays, conventionally carried out by the use of radiation therapy or chemotherapy in addition to invasive operations. However, such therapies place extraordinary stress on the immune system and in some cases can be used to only a limited extent. In addition, most of these forms of therapy require long intervals between the individual treatments in order for the immune system to regenerate. In recent years, therefore, in addition to such “conventional measures”, in particular gene therapeutic approaches or genetic vaccination have been found to be highly promising for treatment or for supporting such therapies. In the case of gene therapeutic approaches, monogenetic diseases are also to the fore, that is to say (inherited) diseases that are caused by a single gene defect and are inherited according to Mendel's laws. The most well known representatives of monogenetic diseases include inter alia mucoviscidosis (cystic fibrosis) and sickle cell anaemia.

Gene therapy and genetic vaccination are molecular medical methods whose use generally in the therapy and prevention of diseases has considerable effects on medical practice. Both methods are based on the introduction of nucleic acids into the patient's cells or tissue and the subsequent processing by the cells or tissue of the information coded for by the nucleic acids that have been introduced, that is to say the expression of the desired polypeptides.

The conventional procedure in current methods of gene therapy and genetic vaccination is the use of DNA for inserting the required genetic information into the cell. Various methods have been developed in this connection for introducing DNA into cells, such as, for example, calcium phosphate transfection, polyprene transfection, protoplast fusion, electroporation, microinjection and lipofection, lipofection in particular having been found to be a suitable method.

A further method that has been proposed in particular in genetic vaccination methods is the use of DNA viruses as DNA vehicles. Such viruses have the advantage that a very high rate of transfection is to be achieved owing to their infectious properties. The viruses that are used are genetically altered so that no functional infectious particles are formed in the transfected cell. Despite this precautionary measure, however, a certain risk of the uncontrolled propagation of the gene-therapeutically active and viral genes that have been introduced cannot be ruled out owing to possible recombination events.

The DNA introduced into the cell is usually integrated to a certain extent into the genome of the transfected cell. On the one hand, this phenomenon can exert a desired effect, because a long-lasting action of the DNA that has been introduced can be achieved thereby. On the other hand, integration into the genome brings a substantial risk for gene therapy. For example, it is possible that the introduced DNA will be inserted into an intact gene, which in turn represents a mutation which impedes or even totally eliminates the function of the endogenous gene. As a result of such integration events, vital enzyme systems for the cell can be eliminated on the one hand, and on the other hand there is also the risk of transformation of the cell so altered into a degenerate state, if a gene critical for the regulation of cell growth is changed by integration of the foreign DNA. For that reason, when using DNA viruses as gene therapeutic agents and as vaccines, a risk, for example of cancer formation, cannot be ruled out. It is also to be noted in this connection that for effective expression of the genes introduced into the cell, the corresponding DNA vehicles contain a strong promoter, for example the viral CMV promoter. The integration of such promoters into the genome of the treated cell can lead to undesirable changes in the regulation of gene expression in the cell.

A further disadvantage of the use of DNA as gene therapeutic agents and as vaccines is the induction of undesired anti-DNA antibodies in the patient, triggering a possible fatal immune response.

In contrast to DNA, the use of RNA as a gene therapeutic agent or as a vaccine is to be categorised as substantially safer. In particular, RNA does not involve the risk of being stably integrated into the genome of the transfected cell. Furthermore, no viral sequences, such as promoters, are required for effective transcription. Moreover, RNA is degraded substantially more simply in vivo. No anti-RNA antibodies have hitherto been detected, presumably because of the relatively short half-life of RNA in the blood circulation as compared with DNA. RNA can therefore be regarded as the molecule of choice for molecular medical methods of therapy.

However, expression systems based on the introduction of nucleic acids into the patient's cells or tissue and the subsequent expression of the desired polypeptides coded for thereby in many cases do not exhibit the desired, or even the required, level of expression in order to enable an effective therapy to be carried out, irrespective of whether DNA or RNA is used.

In the prior art, various different attempts have hitherto been made to increase the yield of the protein expression of expression systems in vitro and/or in vivo. Methods for increasing expression described generally in the prior art are conventionally based on the use of expression cassettes containing specific promoters and correspondingly usable regulation elements. Most such expression cassettes exhibit clear restrictions in transfection owing to their size (independently of the insert used). Furthermore, expression cassettes are typically limited to particular cell systems, so that new expression systems have to be cloned and transfected into the cells in dependence on the cells to be treated. Preference is therefore primarily given first to those nucleic acid molecules which are able to express the encoded proteins in a target cell by systems inherent in the cell, independently of promoters and regulation elements introduced onto expression cassettes.

DE 101 19 005 (Roche Diagnostics GmbH), for example, describes methods of protein expression based on DNA molecules, wherein an improvement in the stability of the linear short DNA is achieved by various measures and consequently improved expression takes place owing to reduced degradation by exonucleases. Accordingly, DE 101 19 005 describes the incorporation of exonuclease-resistant nucleotide analogues or other molecules at the 3′ end of the linear short DNA. In addition, DE 101 19 005 also describes the binding of large molecules to the ends of the linear short DNA, such as, for example, biotin, avidin or streptavidin. Finally, in DE 101 19 005 exonucleases can also be inactivated or inhibited by the addition of competitive or non-competitive inhibitors. However, DE 101 19 005 describes an increase in the expression of the protein only by improving the stability of the linear short DNA that is used. DE 101 19 005 does not show any modifications for RNA, however.

Some measures have additionally been proposed in the prior art for increasing the stability of RNA and thereby permitting its use as a gene therapeutic agent or RNA vaccine. EP-A-1083232 proposes, for example, for solving the problem of the instability of RNA ex vivo, a method for introducing RNA, especially mRNA, into cells and organisms, in which the RNA is present in the form of a complex with a cationic peptide or protein.

Alternatively, WO 99/14346 describes methods for stabilising mRNA, especially modifications of the mRNA, which stabilise the mRNA species against degradation by RNases. Such modifications relate on the one hand to stabilisation by sequence modifications, in particular the reduction of the C and/or U content by base elimination or base substitution. On the other hand, chemical modifications are proposed, such as, for example, the use of nucleotide analogues, as well as 5′- and 3′-blocking groups, an increased length of the poly-A tail and the complexing of the mRNA with stabilising agents, and combinations of the mentioned measures, but without achieving an increase in the expression of the proteins coded for by the mRNAs.

In U.S. Pat. No. 5,580,859 and U.S. Pat. No. 6,214,804, mRNA vaccines and therapeutic agents are disclosed inter alia within the scope of “transient gene therapy” (TGT). Various measures for increasing the translation efficiency and the mRNA stability are described, which measures are based especially on the non-translated sequence regions. However, such modifications require an expression vector that contains a comparatively long untranslated sequence compared with the translated mRNA sequence. This increases the expression vector considerably, however, and may consequently impair the transfection. Furthermore, the sequences described in U.S. Pat. No. 5,580,859 and U.S. Pat. No. 6,214,804 do not exhibit increased expression of the proteins coded for thereby.

Optimised mRNAs are also described in application WO 02/098443 (CureVac GmbH). For example, WO 02/098443 describes mRNAs that are stabilised in general form and optimised for translation in their coding regions and discloses, for example, a method for determining sequence modifications. WO 02/098443 further describes possibilities for substitution of adenosine and uracil nucleotides in mRNA sequences in order to increase the G/C content of the sequences. According to WO 02/098443, such substitutions and adaptations for increasing the G/C content can be used in gene therapeutic applications and also as genetic vaccines for the treatment of cancer. As the base sequence for these modifications, WO 02/098443 generally mentions sequences in which the modified mRNA codes for at least one biologically active peptide or polypeptide which is formed in the patient to be treated, for example, either not at all or inadequately or with faults. Alternatively, WO 02/098443 proposes mRNAs coding for a cancer antigen as the base sequence for such modifications.

Furthermore, it is often found in many methods of the prior art that modifications have to be introduced into gene sequences first by complex and in most cases expensive processes, for example by means of replacement of nucleotides in nucleotide sequences by means of nucleic acid syntheses using DNA/RNA synthesis devices, etc. This generally increases the costs both for studying the stability and expression of modified gene sequences and for the in vitro and in vivo use thereof for the expression of the proteins coded for thereby.

In summary, apart from the use of DNA expression vectors, the prior art does not exhibit a targeted method or uses which deliberately increase the expression of proteins starting from RNA template molecules in vitro or in vivo with a sensible cost/benefit ratio and at the same time maximum variability of the reaction. The object underlying the present invention is, therefore, to provide a method and uses for gene therapy and genetic vaccination which avoid the disadvantages of the use of DNA as a gene therapeutic agent or vaccine and nevertheless, on the basis of mRNA, achieve increased protein expression in the target cell system.

This object is achieved by the use of a base-modified RNA sequence for increasing the expression of a protein, the base-modified RNA sequence containing at least one base modification and coding for a protein. While the present invention relates to the use of the base-modified RNA for increasing the expression level of the encoded protein/peptide, the base-modified RNA as such (containing the (preferred) features disclosed herein alone or in any combination) is also subject-matter of the present invention.

In connection with the present invention, a base-modified RNA used according to the invention comprises any RNA that codes for at least one protein/peptide. The base-modified RNA used according to the invention can be single-stranded or double-stranded, linear or circular or can be in the form of mRNA. The base-modified RNA used according to the invention is particularly preferably in the form of single-stranded RNA, more preferably in the form of mRNA. A base-modified RNA used according to the invention preferably has a length of from 50 to 15,000 nucleotides, more preferably a length of from 50 to 10,000 nucleotides, yet more preferably a length of from 500 to 10,000 nucleotides and most preferably a length of from 500 to 5000 nucleotides. Most preferably, the inventive base-modified RNA codes for at least one protein/peptide sequence. In this context, a coding RNA is typically an mRNA, which is composed of several structural elements, e.g. an optional 5′-UTR region, an upstream positioned ribosomal binding site followed by a coding region, an optional 3′-UTR region, which may be followed by a poly-A tail (and/or a poly-C-tail).

The base-modified RNA sequence used according to the invention typically contains at least one base modification, which is preferably suitable for increasing the expression of the protein coded for by the RNA significantly as compared with the unaltered, i.e. natural (=native), RNA sequence. Significant in this case means an increase in the expression of the protein compared with the expression of the native RNA sequence by at least 20%, preferably at least 30%, 40%, 50% or 60%, more preferably by at least 70%, 80%, 90% or even 100% and most preferably by at least 150%, 200% or even 300%. In connection with the present invention, a nucleotide having a base modification of the base-modified RNA used according to the invention is preferably selected from the group of the base-modified nucleotides consisting of:
2-amino-6-chloropurineriboside-5′-triphosphate
2-aminoadenosine-5′-triphosphate
2-thiocytidine-5′-triphosphate
2-thiouridine-5′-triphosphate
4-thiouridine-5′-triphosphate
5-aminoallylcytidine-5′-triphosphate
5-aminoallyluridine-5′-triphosphate
5-bromocytidine-5′-triphosphate
5-bromouridine-5′-triphosphate
5-iodocytidine-5′-triphosphate
5-iodouridine-5′-triphosphate
5-methylcytidine-5′-triphosphate
5-methyluridine-5′-triphosphate
6-azacytidine-5′-triphosphate
6-azauridine-5′-triphosphate
6-chloropurineriboside-5′-triphosphate
7-deazaadenosine-5′-triphosphate
7-deazaguanosine-5′-triphosphate
8-azaadenosine-5′-triphosphate
8-azidoadenosine-5′-triphosphate
benzimidazole-riboside-5′-triphosphate
N1-methyladenosine-5′-triphosphate
N1-methylguanosine-5′-triphosphate
N6-methyladenosine-5′-triphosphate
O6-methylguanosine-5′-triphosphate
pseudouridine-5′-triphosphate
puromycin-5′-triphosphate
xanthosine-5′-triphosphate

Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In this connection, without being limited thereto, the inventors attribute an increase in the expression of the protein coded for by the base-modified RNA inter alia to the improvement in the stabilisation of secondary structures and optionally to the resulting “more rigid” structure of the RNA and the increased “base stacking”. For example, pseudouridine-5′-triphosphate is known to occur naturally in structural RNAs (tRNA, rRNA and snRNA) in both eukaryotes and prokaryotes. It is assumed in this connection that pseudouridine is necessary in rRNA for stabilising secondary structures. In the course of evolution, the amount of pseudouridine in the RNA has increased and it has been possible to show, surprisingly, that translation is dependent on the presence of pseudouridine in the tRNA and rRNA, the interaction between tRNA and mRNA presumably being increased thereby. The conversion of uridine to pseudouridine takes place posttranscriptionally by pseudouridine synthase. In the case of 5-methylcytidine-5′-triphosphate, a posttranscriptional modification of RNA also takes place, which is catalysed by methyltransferases. A further increase in the amount of pseudouridine and the base modification of other nucleotides presumably leads to similar effects, which, unlike the naturally occurring increased amounts of pseudouridine in the sequence, can be carried out in a targeted manner and with substantially greater variability. A similar mechanism as for pseudouridine-5′-triphosphate is therefore assumed for 5-methylcytidine-5′-triphosphate and the other base modifications mentioned herein, that is to say an improved stabilisation of secondary structures and, based thereon, an improved translation efficiency. In addition to this structurally based increase in expression, however, a positive effect on translation is also supposed independently of the stabilisation of secondary structures and a “more rigid” structure of the RNA. Further causes of increased expression are optionally also the lower rate of degradation of the mRNA sequences by RNAses in vitro or in vivo.

The base modification(s) of the RNA used according to the invention can be introduced into the RNA by means of methods known to a person skilled in the art. Suitable methods are, for example, synthesis methods using (automatic or semi-automatic) oligonucleotide synthesis devices, biochemical methods, such as, for example, in vitro transcription methods, etc. In this connection there can preferably be used in the case of (relatively short) sequences, whose length generally does not exceed from 50 to 100 nucleotides, synthesis methods using (automatic or semi-automatic) oligonucleotide synthesis devices as well as in vitro transcription methods. In the case of (relatively long) sequences, for example sequences having a length of more than 50 to 100 nucleotides, biochemical methods are preferably suitable, such as, for example, in vitro transcription methods, preferably an in vitro transcription method according to the invention as described hereinbelow. However, even longer base-modified RNA molecules may be synthesized synthetically by coupling various synthesized fragments covalently.

Base modifications of base-modified RNA sequences used according to the invention typically occur on at least one (base-modifiable) nucleotide of the base-modified RNA sequence, preferably on at least 2, 3, 4, 5, 6, 7, 8, 9 or 10 (base-modifiable) nucleotides, more preferably on at least 10 to 20 (base-modifiable) nucleotides, yet more preferably on at least 10 to 100 (base-modifiable) nucleotides and most preferably on at least 10 to 200 or more (base-modifiable) nucleotides. In other words, base modifications in a base-modified RNA sequence used according to the invention typically occur on at least one (base-modifiable) nucleotide of the base-modified RNA sequence, preferably on at least 10% of all (base-modifiable) nucleotides, more preferably on at least 25% of all (base-modifiable) nucleotides, yet more preferably on at least 50% of all (base-modifiable) nucleotides, even more preferably on at least 75% of all (base-modifiable) nucleotides and most preferably on 100% of the (base-modifiable) nucleotides contained in the base-modified RNA sequence used according to the invention. The above preferred percentage values may also hold for the coding region(s) of the base-modified RNA, that is e.g. preferably 10%, more preferably 25%, more preferably at least 50%, more preferably at least 75% and etc. of the nucleotides of the coding region of the base-modified RNA may be substituted.

A “base-modifiable nucleotide” in this connection is any (preferably naturally occurring (natural, native) and hence unmodified) nucleotide that is to be replaced by a base-modified nucleotide as described above. It is thereby possible for all the nucleotides of the RNA sequence to be base-modified, or only specific chosen nucleotides of the RNA sequence. If all the nucleotides of the RNA sequence are to be base-modified, then 100% of the “base-modifiable nucleotides” of the RNA sequence are all the nucleotides of the RNA sequence used. If, on the other hand, only particular chosen nucleotides of the RNA sequence are base-modified, then the chosen nucleotides are, for example, adenosine, cytidine, guanosine or uridine. Thus, for example, an adenosine of the native sequence can be replaced by a base-modified adenosine, a cytidine can be replaced by a base-modified cytidine, a uridine by a base-modified uridine and a guanosine by a base-modified guanosine. In this case, 100% of the “base-modifiable nucleotides” of the RNA sequence are 100% of the adenosines, cytidines, guanosines or uridines contained in the RNA sequence used.

Preferred embodiments of the base-modified RNA of the present invention may e.g. contain at least 10% of all RNA cytidine-5′-triphosphate nucleotides (or all cytidine-5′-triphosphate nucleotides of the coding region) modified to base-modified cytidine nucleotides, e.g. 5-methylcytidine-5′-triphosphate and/or 5-bromocytidine-5′-triphosphate nucleotides, and/or at least 10% of all guanosine-5′-triphosphate nucleotides (or all guanosine-5′-triphosphate nucleotides of the coding region) modified to base-modified guanosine nucleotides, e.g. 7-deazaguanosine-5′-triphosphate nucleotides, and/or at least 10% of all uridine-5′-triphosphate nucleotides (or all uridine-5′-triphosphate nucleotides of the coding region) modified to base-modified uridine nucleotides, e.g. pseudouridine-5′-triphosphate nucleotides. Another preferred embodiment of the base-modified RNA of the present invention may e.g. contain at least 25% of all RNA cytidine-5′-triphosphate nucleotides (or all cytidine-5′-triphosphate nucleotides of the coding region) modified to base-modified cytidine nucleotides, e.g. 5-methylcytidine-5′-triphosphate and/or 5-bromocytidine-5′-triphosphate nucleotides, and/or at least 25% of all guanosine-5′-triphosphate nucleotides (or all guanosine-5′-triphosphate nucleotides of the coding region) modified to base-modified guanosine nucleotides, e.g. 7-deazaguanosine-5′-triphosphate nucleotides, and/or at least 25% of all uridine-5′-triphosphate nucleotides (or all uridine-5′-triphosphate nucleotides of the coding region) modified to base-modified uridine nucleotides, e.g. pseudouridine-5′-triphosphate nucleotides. Another preferred embodiment of the base-modified RNA of the present invention may e.g. contain at least 50% of all RNA cytidine-5′-triphosphate nucleotides (or all cytidine-5′-triphosphate nucleotides of the coding region) modified to base-modified cytidine nucleotides, e.g. 5-methylcytidine-5′-triphosphate and/or 5-bromocytidine-5′-triphosphate nucleotides, and/or at least 50% of all guanosine-5′-triphosphate nucleotides (or all guanosine-5′-triphosphate nucleotides of the coding region) modified to base-modified guanosine nucleotides, e.g. 7-deazaguanosine-5′-triphosphate nucleotides, and/or at least 50% of all uridine-5′-triphosphate nucleotides (or all uridine-5′-triphosphate nucleotides of the coding region) modified to base-modified uridine nucleotides, e.g. pseudouridine-5′-triphosphate nucleotides. Another preferred embodiment of the base-modified RNA of the present invention may e.g. contain at least 75% of all RNA cytidine-5′-triphosphate nucleotides (or all cytidine-5′-triphosphate nucleotides of the coding region) modified to base-modified cytidine nucleotides, e.g. 5-methylcytidine-5′-triphosphate and/or 5-bromocytidine-5′-triphosphate nucleotides, and/or at least 75% of all guanosine-5′-triphosphate nucleotides (or all guanosine-5′-triphosphate nucleotides of the coding region) modified to base-modified guanosine nucleotides, e.g. 7-deazaguanosine-5′-triphosphate nucleotides, and/or at least 75% of all uridine-5′-triphosphate nucleotides (or all uridine-5′-triphosphate nucleotides of the coding region) modified to base-modified uridine nucleotides, e.g. pseudouridine-5′-triphosphate nucleotides. Specifically preferred embodiments are those, wherein the coding region of the base-modified RNA contain at least 75%, more preferably at least 85% more preferably at least 90% and most preferably at least 95% base-modified nucleotides of one specific type, that means that e.g. at least 75%, 85%, 90%, 95% of all uridine nucleotides are substituted by base-modified uridine nucleotides, e.g. pseudouridine-5′-triphosphate nucleotides or combinations of pseudouridine-5′-triphosphate nucleotides with at least one other type of base-modified uridine nucleotides, or that e.g. at least 75%, 85%, 90%, 95% of all cytidine nucleotides are substituted by base-modified cytidine nucleotides, e.g. 5-methylcytidine-5′-triphosphate and/or 5-bromocytidine-5′-triphosphate nucleotides or combinations of 5-methylcytidine-5′-triphosphate and/or 5-bromocytidine-5′-triphosphate nucleotides with at least one other type of base-modified cytidine nucleotides, or that e.g. at least 75%, 85%, 90%, 95% of all adenosine nucleotides are substituted by base-modified adenosine nucleotides or combinations of at least two types of base-modified adenosine nucleotides or that e.g. at least 75%, 85%, 90%, 95% of all guanosine nucleotides are substituted by base-modified guanosine nucleotides, e.g. 7-deazaguanosine-5′-triphosphate nucleotides or combinations of deazaguanosine-5′-triphosphate nucleotides with at least one other type of base-modified guanosine nucleotides.

Base-modified RNA sequences used according to the invention can further also contain backbone modifications. A backbone modification in connection with the present invention is a modification in which phosphates of the backbone of the nucleotides contained in the RNA are chemically modified. Such backbone modifications typically include, without implying any limitation, modifications from the group consisting of methylphosphonates, phosphoramidates and phosphorothioates (e.g. cytidine-5′-O-(1-thiophosphate)).

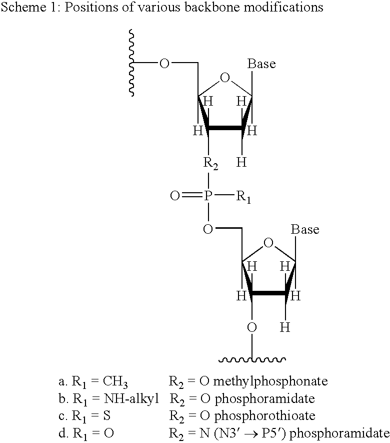

Base-modified RNA sequences used according to the invention can likewise also contain sugar modifications. A sugar modification in connection with the present invention is a chemical modification of the sugar of the nucleotides present and typically includes, without implying any limitation, sugar modifications selected from the group consisting of 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), or azidotriphosphate (2′-azido-2′-deoxycytidine-5′-triphosphate, 2′-azido-2′-deoxyuridine-5′-triphosphate).

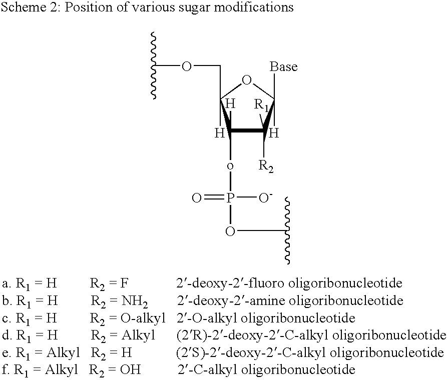

The base-modified RNA sequence used according to the invention preferably does not contain any sugar modifications or backbone modifications, however. The reason for this preferred exclusion is that particular backbone modifications and sugar modifications of RNA sequences can on the one hand prevent or at least greatly reduce their in vitro transcription. Thus, an in vitro transcription of eGFP carried out by way of example functions, for example, only with the sugar modifications 2′-amino-2′-deoxyuridine-5′-phosphate, 2′-fluoro-2′-deoxyuridine-5′-phosphate and 2′-azido-2′-deoxyuridine-5′-phosphate. In addition, the translation of the protein, that is to say protein expression in vitro or in vivo, is typically considerably reduced by backbone modifications and, independently thereof, by sugar modifications of RNA sequences. It has been possible to demonstrate this, for eGFP, for example, in connection with the backbone modifications and sugar modifications chosen above.

According to an preferred embodiment, the base-modified RNA used according to the invention has a GC content that has been changed as compared with the native sequence. According to a first alternative of the base-modified RNA used according to the invention, the G/C content for the coding region of the base-modified RNA is greater than the G/C content for the coding region of the native RNA sequence, the amino acid sequence that is coded for being unchanged as compared with the wild type, that is to say the amino acid sequence coded for by the native RNA sequence. The composition and the sequence of the various nucleotides play a large part here. In particular, sequences having an increased G(guanine)/C(cytosine) content are more stable than sequences having an increased A(adenine)/U(uracil) content. Therefore, according to the invention, the codons are varied as compared with the wild type, while retaining the translated amino acid sequence, in such a manner that they contain an increased number of G/C nucleotides. Because several codons code for the same amino acid (degeneracy of the genetic code), the codons advantageous for stability can be determined (alternative codon usage).

In dependence on the amino acid sequence to be coded for by the base-modified RNA used according to the invention, different possibilities for modifying the native sequence of the base-modified RNA used according to the invention are possible. In the case of amino acids coded for by codons that contain solely G or C nucleotides, no modification of the codon is required. Accordingly, the codons for Pro (CCC or CCG), Arg (CGC or CGG), Ala (GCC or GCG) and Gly (GGC or GGG) do not require any change because no A or U is present.

In the following cases, the codons containing A and/or U nucleotides are changed by substitution with different codons that code for the same amino acids but do not contain A and/or U. Examples are:

the codons for Pro can be changed from CCU or CCA to CCC or CCG;
the codons for Arg can be changed from CGU or CGA or AGA or AGG to CGC or CGG;
the codons for Ala can be changed from GCU or GCA to GCC or GCG;
the codons for Gly can be changed from GGU or GGA to GGC or GGG.

In other cases, although A or U nucleotides cannot be eliminated from the codons, it is possible to reduce the A and U content by the use of codons containing fewer A and/or U nucleotides. For example:

the codons for Phe can be changed from UUU to UUC;
the codons for Leu can be changed from UUA, CUU or CUA to CUC or CUG;
the codons for Ser can be changed from UCU or UCA or AGU to UCC, UCG or AGC;
the codon for Tyr can be changed from UAU to UAC;
the stop codon UAA can be changed to UAG or UGA;
the codon for Cys can be changed from UGU to UGC;
the codon His can be changed from CAU to CAC;
the codon for Gln can be changed from CAA to CAG;
the codons for Ile can be changed from AUU or AUA to AUC;
the codons for Thr can be changed from ACU or ACA to ACC or ACG;
the codon for Asn can be changed from AAU to AAC;
the codon for Lys can be changed from AAA to AAG;
the codons for Val can be changed from GUU or GUA to GUC or GUG;
the codon for Asp can be changed from GAU to GAC;
the codon for Glu can be changed from GAA to GAG.

In the case of the codons for Met (AUG) and Trp (UGG), on the other hand, there is no possibility of sequence modification.

The substitutions listed above can, of course, be used individually or in all possible combinations for increasing the G/C content of the base-modified RNA used according to the invention as compared with the native RNA sequence (or nucleic acid sequence). For example, all the codons for Thr occurring in the native RNA sequence can be changed to ACC (or ACG). Preferably, however, combinations of the above substitution possibilities are used, for example:

substitution of all codons coding for Thr in the native RNA sequence by ACC (or ACG) and
substitution of all codons originally coding for Ser by UCC (or UCG or AGC);
substitution of all codons coding for Ile in the native RNA sequence by AUC and substitution of all codons originally coding for Lys by AAG and substitution of all codons originally coding for Tyr by UAC;
substitution of all codons coding for Val in the native RNA sequence by GUC (or GUG) and
substitution of all codons originally coding for Glu by GAG and substitution of all codons originally coding for Ala by GCC (or GCG) and substitution of all codons originally coding for Arg by CGC (or CGG);
substitution of all codons coding for Val in the native RNA sequence by GUC (or GUG) and
substitution of all codons originally coding for Glu by GAG and substitution of all codons originally coding for Ala by GCC (or GCG) and substitution of all codons originally coding for Gly by GGC (or GGG) and substitution of all codons originally coding for Asn by AAC;
substitution of all codons coding for Val in the native RNA sequence by GUC (or GUG) and substitution of all codons originally coding for Phe by UUC and substitution of all codons originally coding for Cys by UGC and substitution of all codons originally coding for Leu by CUG (or CUC) and substitution of all codons originally coding for Gln by CAG and substitution of all codons originally coding for Pro by CCC (or CCG);
etc.

The G/C content of the coding region of the base-modified RNA used according to the invention is preferably increased as compared with the G/C content of the coding region of the native RNA in such a manner that at least 5%, at least 10%, at least 15%, at least 20%, at least 25% or more preferably at least 30%, at least 35%, at least 40%, at least 45%, at least 50% or at least 55%, yet more preferably at least 60%, at least 65%, at least 70% or at least 75% and most preferably at least 80%, at least 85%, at least 90%, at least 95% or at least 100% of the possible modifiable codons of the coding region of the native RNA (or nucleic acid) are modified.

It is particularly preferred in this connection to increase the G/C content of the base-modified RNA used according to the invention, in particular in the coding region, as much as possible compared with the native RNA sequence. The G/C modified RNA may preferably be provided such that at least 10%, preferably at least 20%, more preferably at least 59%, more preferably at least 75% and more preferably at least 90% of the substituted G/C nucleotides introduced according to this modification are base-modified G and/or C nucleotides, e.g. 7-deazaguanosine-5′-triphosphate nucleotides and/or 5-methylcytidine-5′-triphosphate and/or 5-bromocytidine-5′-triphosphate nucleotides.

A second alternative of the base-modified RNA used according to the invention is based on the finding that the translation efficiency of the RNA is also determined by a varying frequency in the occurrence of tRNAs in cells. If, therefore, so-called “rare” codons are present in an increased number in a RNA sequence, then the corresponding RNA is translated markedly more poorly than in the case where codons coding for relative “frequent” tRNAs are present.

According to this second alternative of the base-modified RNA used according to the invention, therefore, the coding region of the base-modified RNA used according to the invention is changed as compared with the coding region of the native RNA in such a manner that at least one codon of the native RNA coding for a tRNA that is relatively rare in the cell is replaced by a codon coding for a tRNA that is relatively frequent in the cell and that carries the same amino acid as the relatively rare tRNA.

By means of this modification, the base-modified RNA sequence used according to the invention is modified in such a manner that codons for which frequently occurring tRNAs are available are inserted. Which tRNAs occur relatively frequently in the cell and which, by contrast, are relatively rare is known to a person skilled in the art; see, for example, Akashi, Curr. Opin. Genet. Dev. 2001, 11(6): 660-666.

By means of this modification, all the codons of the base-modified RNA sequence used according to the invention that code for a tRNA that is relatively rare in the cell can be replaced according to the invention by a codon that codes for a tRNA that is relatively frequent in the cell and that carries the same amino acid as the relatively rare tRNA.

It is particularly preferred to link the increased, especially maximum, sequential G/C content in the base-modified RNA used according to the invention with the “frequent” codons, without changing the amino acid sequence coded for by the base-modified RNA used according to the invention. This preferred embodiment represents a particularly efficient translated and stabilised base-modified RNA used according to the invention (for example for a pharmaceutical composition according to the invention).

The above-mentioned embodiments of the base-modified RNA used according to the invention can be combined with one another in a suitable manner. Determination of the optimum base-modified RNA used according to the invention can be carried out by methods known to the person skilled in the art, for example manually and/or by means of an automated method, as disclosed according to WO 02/098443. Adaptation of the RNA sequences can thereby be carried out with the additional different optimisation aims described above: On the one hand with maximum G/C content, on the other hand while taking the best possible account of the frequency of the tRNAs according to codon usage. In the first step of the method, a virtual translation of any desired RNA (or DNA) sequence is carried out in order to generate the corresponding amino acid sequence. Starting from the amino acid sequence, a virtual reverse translation is carried out which, on the basis of the degeneracy of the genetic code, yields choice possibilities for the corresponding codons. Depending on the required optimisation or modification, corresponding selection lists and optimisation algorithms are used to choose the suitable codons. The algorithms are typically executed by means of suitable software on a computer. For example, the optimised RNA sequence is prepared and can be given out by means of a suitable display device, for example, and compared with the original (wild-type) sequence. The same is also true of the frequency of the individual nucleotides. The changes as compared with the original nucleotide sequence are preferably emphasised. Furthermore, according to a preferred embodiment, stable sequences known in nature are read in, which sequences can form the basis for a RNA stabilised according to native sequence motifs. It is likewise possible to provide a secondary structural analysis, which is able to analyse stabilising and destabilising properties or regions of the RNA on the basis of structural calculations.

In the sequences of eukaryotic RNAs there are typically destabilising sequence elements (DSEs), to which signal proteins bind and regulate the enzymatic degradation of the RNA in vivo. Therefore, in order further to stabilise the base-modified RNA used according to the invention, optionally in the region coding for the protein, one or more changes are preferably made as compared with the corresponding region of the native RNA, so that no destabilising sequence elements are present. Of course, it is likewise preferred according to the invention to eliminate DSEs optionally present in the untranslated regions (3′- and/or 5′-UTR) from the RNA.

Examples of such destabilising sequences are AU-rich sequences (“AURES”), which occur in 3′-UTR sections of numerous unstable RNAs (Caput et al., Proc. Natl. Acad. Sci. USA 1986, 83: 1670 to 1674). The base-modified RNA used according to the invention is therefore preferably changed as compared with the native RNA in such a manner that it does not contain any such destabilising sequences. This is also true of those sequence motifs that are recognised by possible endonucleases, for example the sequence GAACAAG, which is contained in the 3′-UTR segment of the gene coding for the transferrin receptor (Binder et al., EMBO J. 1994, 13: 1969 to 1980). Such sequence motifs are preferably also eliminated from the base-modified RNA used according to the invention.

Various methods are known to a person skilled in the art that are suitable for the substitution of codons in RNAs, that is to say for the substitution of codons in the base-modified RNA used according to the invention. In the case of relatively short coding regions (that code for biologically active or antigenic peptides), it is possible, for example, to synthesise the entire base-modified RNA used according to the invention chemically using standard techniques as are known to a person skilled in the art.

It is preferred, however, to introduce base substitutions using a DNA matrix for preparing the base-modified RNA used according to the invention by means of techniques of targeted mutagenesis (see e.g. Maniatis et al., Molecular Cloning: A Laboratory Manual, Cold Spring Harbor Laboratory Press, 3rd Ed., Cold Spring Harbor, N.Y., 2001). In this process, a corresponding DNA molecule is therefore transcribed in vitro (see below) to produce the base-modified RNA used according to the invention. This DNA matrix optionally possesses a suitable promoter, for example a T3, T7 or SP6 promoter, for in vitro transcription, followed by the desired nucleotide sequence for the base-modified RNA to be prepared and a termination signal for the in vitro transcription. The DNA molecule that forms the matrix of the base-modified RNA construct to be produced can then be prepared by fermentative propagation and subsequent isolation as part of a plasmid replicable in bacteria. As plasmids suitable therefor there may be mentioned, for example, the plasmids pT7Ts (GenBank accession number U26404; Lai et al., Development 1995, 121: 2349 to 2360), pGEM® series, for example pGEM®-1 (GenBank accession number X65300; from Promega) and pSP64 (GenBank accession number X65327); see also Mezei and Storts, Purification of PCR Products, in: Griffin and Griffin (eds.), PCR Technology: Current Innovation, CRC Press, Boca Raton, Fla., 2001.

It is thus possible using short synthetic DNA oligonucleotides that have short single-stranded transitions at the cleavage sites, or genes prepared by chemical synthesis, to clone the desired nucleotide sequence into a suitable plasmid by molecular biological methods known to a person skilled in the art (see Maniatis et al., (2001) supra). The DNA molecule is then cut out of the plasmid, in which it can be present in a single copy or multiple copies, by digestion with restriction endonucleases.

According to a particular embodiment of the present invention, the base-modified RNA used according to the invention can additionally have a 5′-cap structure (a modified guanosine nucleotide). As examples of cap structures there may be mentioned, without being limited thereto, m7G(5′)ppp(5′(A,G(5′)ppp(5′)A and G(5′)ppp(5′)G.

According to a further preferred embodiment of the present invention, the base-modified RNA used according to the invention contains a poly-A tail of at least about 50 nucleotides, preferably of at least about 70 nucleotides, more preferably of at least about 100 nucleotides and yet more preferably of at least about 200 nucleotides.

According to another preferred embodiment of the present invention, the base-modified RNA used according to the invention contains a poly-C tail of at least about 20 nucleotides, preferably of at least about 30 nucleotides, more preferably of at least about 40 nucleotides and yet more preferably of at least about 50 nucleotides.

According to a further embodiment, the base-modified RNA used according to the invention, as described above, can further contain a nucleic acid section that codes for a tag for purification. Such tags include, without implying any limitation, for example a hexahistidine tag (HIS tag, polyhistidine tag), a streptavidin tag (strep tag), a SBP tag (streptavidin binding tag), a GST (glutathione S-transferase) tag, etc. The base-modified RNA can further code for a tag for purification via an antibody epitope (antibody binding tag), for example a Myc tag, a Swal 1 epitope, FLAG tag, a Ha tag, etc., that is to say by recognition of the epitope via the (immobilised) antibody.

For an efficient translation of RNA, in particular mRNA, effective binding of the ribosomes to the ribosome binding site (Kozak sequence: GCCGCCACCAUGG (SEQ ID NO: 1), the AUG forms the start codon) is also necessary. It has been noted in this respect that an increased A/U content around this site permits more efficient ribosome binding to the mRNA. Therefore, according to another preferred embodiment of the present invention, the base-modified RNA used according to the invention can have an increased A/U content around the ribosome binding site, preferably an A/U content increased by from 5 to 50%, more preferably by from 25 to 50% or more, as compared with the native RNA.

Furthermore, it is possible according to an embodiment of the base-modified RNA used according to the invention to introduce one or more so-called IRESs (internal ribosomal entry side) into the RNA. An IRES can thus function as the only ribosomal binding site, but it can also serve to provide a base-modified RNA used according to the invention that codes for a plurality of proteins which are to be translated independently of one another by the ribosomes (“multicistronic RNA”). Examples of IRES sequences which can be used according to the invention are those from picorna viruses (e.g. FMDV), plague viruses (CFFV), polio viruses (PV), encephalo-myocarditis viruses (ECMV), foot-and-mouth viruses (FMDV), hepatitis C viruses (HCV), conventional swine fever viruses (CSFV), murine leukoma virus (MLV), simean immune deficiency virus (SIV) or cricket paralysis viruses (CrPV).

According to a further preferred embodiment of the present invention, the base-modified RNA used according to the invention contains in its 5′- and/or 3′-untranslated regions stabilising sequences that are capable of increasing the half-life of the RNA in the cytosol. These stabilising sequences can exhibit 100% sequence homology with naturally occurring sequences that occur in viruses, bacteria and eukaryotes, but they can also be partially or wholly of synthetic nature. As examples of stabilising sequences which can be used in the present invention there may be mentioned the untranslated sequences (UTR) of the β-globin gene, for example of Homo sapiens or Xenopus laevis. Another example of a stabilising sequence has the general formula (C/U)CCANₓCCC(U/A)PyₓUC(C/U)CC (SEQ ID NO: 2), which is contained in the 3′-UTR of the very stable RNA that codes for α-globin, α-(I)-collagen, 15-lipoxygenase or for tyrosine-hydroxylase (see Holcik et al., Proc. Natl. Acad. Sci. USA 1997, 94: 2410 to 2414). Of course, such stabilising sequences can be used individually or in combination with one another as well as in combination with other stabilising sequences known to a person skilled in the art.

Furthermore, in a preferred embodiment the effective transfer of the base-modified RNA used according to the invention into the cells to be treated or the organism to be treated can be improved by associating the base-modified RNA used according to the invention with a cationic peptide or protein or binding it thereto. In particular the use of protamine, histone, spermin or nucleoline or derivatives of those sequences containing the basic nucleic acid binding sequence as the polycationic, nucleic-acid-binding protein is particularly effective.

Furthermore, the use of other cationic peptides or proteins, such as poly-L-lysine or histones, is likewise possible. This procedure for stabilising the modified RNA is described, for example, in EP-A-1083232, the disclosure of which is incorporated by reference into the present invention in its entirety.

In connection with the present invention, the protein coded for by the base-modified RNA used according to the invention can be selected preferably from all therapeutically useful proteins, for example from all proteins known to a person skilled in the art that are produced by recombinant methods or occur naturally and that are used for therapeutic purposes, for diagnostic purposes. In addition the present invention provides a system by the base-modified RNA which allows to express protein with an increase expression rate which is useful for almost any purpose, e.g. for diagnostic or for research purposes. Accordingly, the inventive base-modified RNA may encode almost any protein, which shall be expressed with a higher expression rate in an in vitro or in vivo expression system than the corresponding naturally occurring RNA (without base-modified nucleotides).

The protein to be encoded by the base-modified inventive RNA may e.g. be selected from any of the proteins given in the following: 0ATL3, 0FC3, 0PA3, 0PD2, 4-1BBL, 5T4, 6Ckine, 707-AP, 9D7, A2M, AA, AAAS, AACT, AASS, ABAT, ABCA1, ABCA4, ABCB1, ABCB11, ABCB2, ABCB4, ABCB7, ABCC2, ABCC6, ABCC8, ABCD1, ABCD3, ABCG5, ABCG8, ABL1, ABO, ABR ACAA1, ACACA, ACADL, ACADM, ACADS, ACADVL, ACAT1, ACCPN, ACE, ACHE, ACHM3, ACHM1, ACLS, ACPI, ACTA1, ACTC, ACTN4, ACVRL1, AD2, ADA, ADAMTS13, ADAMTS2, ADFN, ADH1B, ADH1C, ADLDH3A2, ADRB2, ADRB3, ADSL, AEZ, AFA, AFD1, AFP, AGA, AGL, AGMX2, AGPS, AGS1, AGT, AGTR1, AGXT, AH02, AHCY, AHDS, AHHR, AHSG, AIC, AIED, AIH2, AIH3, AIM-2, AIPL1, AIRE, AK1, ALAD, ALAS2, ALB, HPG1, ALDH2, ALDH3A2, ALDH4A1, ALDH5A1, ALDH1A1, ALDOA, ALDOB, ALMS1, ALPL, ALPP, ALS2, ALX4, AMACR, AMBP, AMCD, AMCD1, AMCN, AMELX, AMELY, AMGL, AMH, AMHR2, AMPD3, AMPD1, AMT, ANC, ANCR, ANK1, ANOP1, AOM, AP0A4, AP0C2, AP0C3, AP3B1, APC, aPKC, APOA2, APOA1, APOB, APOC3, APOC2, APOE, APOH, APP, APRT, APS1, AQP2, AR, ARAF1, ARG1, ARHGEF12, ARMET, ARSA, ARSB, ARSC2, ARSE, ART-4, ARTC1/m, ARTS, ARVD1, ARX, AS, ASAH, ASAT, ASD1, ASL, ASMD, ASMT, ASNS, ASPA, ASS, ASSP2, ASSP5, ASSP6, AT3, ATD, ATHS, ATM, ATP2A1, ATP2A2, ATP2C1, ATP6B1, ATP7A, ATP7B, ATP8B1, ATPSK2, ATRX, ATXN1, ATXN2, ATXN3, AUTS1, AVMD, AVP, AVPR2, AVSD1, AXIN1, AXIN2, AZF2, B2M, B4GALT7, B7H4, BAGE, BAGE-1, BAX, BBS2, BBS3, BBS4, BCA225, BCAA, BCH, BCHE, BCKDHA, BCKDHB, BCL10, BCL2, BCL3, BCL5, BCL6, BCPM, BCR, BCR/ABL, BDC, BDE, BDMF, BDMR, BEST1, beta-Catenin/m, BF, BFHD, BFIC, BFLS, BFSP2, BGLAP, BGN, BHD, BHR1, BING-4, BIRC5, BJS, BLM, BLMH, BLNK, BMPR2, BPGM, BRAF, BRCA1, BRCA1/m, BRCA2, BRCA2/m, BRCD2, BRCD1, BRDT, BSCL, BSCL2, BTAA, BTD, BTK, BUB1, BWS, BZX, C0L2A1, C0L6A1, C1NH, C1QA, C1QB, C1QG, C1S, C2, C3, C4A, C4B, C5, C6, C7, C7orf2, C8A, C8B, C9, CA125, CA15-3/CA 27-29, CA195, CA19-9, CA72-4, CA2, CA242, CA50, CABYR, CACD, CACNA2D1, CACNA1A, CACNA1F, CACNA1S, CACNB2, CACNB4, CAGE, CA1, CALB3, CALCA, CALCR, CALM, CALR, CAM43, CAMEL, CAP-1, CAPN3, CARD15, CASP-5/m, CASP-8, CASP-8/m, CASR, CAT, CATM, CAV3, CB1, CBBM, CBS, CCA1, CCAL2, CCAL1, CCAT, CCL-1, CCL-11, CCL-12, CCL-13, CCL-14, CCL-15, CCL-16, CCL-17, CCL-18, CCL-19, CCL-2, CCL-20, CCL-21, CCL-22, CCL-23, CCL-24, CCL-25, CCL-27, CCL-3, CCL-4, CCL-5, CCL-7, CCL-8, CCM1, CCNB1, CCND1, CCO, CCR2, CCR5, CCT, CCV, CCZS, CD1, CD19, CD20, CD22, CD25, CD27, CD27L, cD3, CD30, CD30, CD30L, CD33, CD36, CD3E, CD3G, CD3Z, CD4, CD40, CD40L, CD44, CD44v, CD44v6, CD52, CD55, CD56, CD59, CD80, CD86, CDAN1, CDAN2, CDAN3, CDC27, CDC27/m, CDC2L1, CDH1, CDK4, CDK4/m, CDKN1C, CDKN2A, CDKN2A/m, CDKN1A, CDKN1C, CDL1, CDPD1, CDR1, CEA, CEACAM1, CEACAM5, CECR, CECR9, CEPA, CETP, CFNS, CFTR, CGF1, CHAC, CHED2, CHED1, CHEK2, CHM, CHML, CHR39C, CHRNA4, CHRNA1, CHRNB1, CHRNE, CHS, CHS1, CHST6, CHX10, CIAS1, CIDX, CKN1, CLA2, CLA3, CLA1, CLCA2, CLCN1, CLCN5, CLCNKB, CLDN16, CLP, CLN2, CLN3, CLN4, CLN5, CLN6, CLN8, C1QA, C1QB, C1QG, C1R, CLS, CMCWTD, CMDJ, CMD1A, CMD1B, CMH2, MH3, CMH6, CMKBR2, CMKBR5, CML28, CML66, CMM, CMT2B, CMT2D, CMT4A, CMT1A, CMTX2, CMTX3, C-MYC, CNA1, CND, CNGA3, CNGA1, CNGB3, CNSN, CNTF, COA-1/m, COCH, COD2, COD1, COH1, COL10A, COL2A2, COL11A2, COL17A1, COL1A1, COL1A2, COL2A1, COL3A1, COL4A3, COL4A4, COL4A5, COL4A6, COL5A1, COL5A2, COL6A1, COL6A2, COL6A3, COL7A1, COL8A2, COL9A2, COL9A3, COL11A1, COL1A2, COL23A1, COL1A1, COLQ, COMP, COMT, CORD5, CORD1, COX10, COX-2, CP, CPB2, CPO, CPP, CPS1, CPT2, CPT1A, CPX, CRAT, CRB1, CRBM, CREBBP, CRH, CRHBP, CRS, CRV, CRX, CRYAB, CRYBA1, CRYBB2, CRYGA, CRYGC, CRYGD, CSA, CSE, CSF1R, CSF2RA, CSF2RB, CSF3R, CSF1R, CST3, CSTB, CT, CT7, CT-9/BRD6, CTAA1, CTACK, CTEN, CTH, CTHM, CTLA4, CTM, CTNNB1, CTNS, CTPA, CTSB, CTSC, CTSK, CTSL, CTS1, CUBN, CVD1, CX3CL1, CXCL1, CXCL10, CXCL1-1, CXCL12, CXCL13, CXCL16, CXCL2, CXCL3, CXCL4, CXCL5, CXCL6, CXCL7, CXCL8, CXCL9, CYB5, CYBA, CYBB, CYBB5, CYFRA 21-1, CYLD, CYLD1, CYMD, CYP11B1, CYP11B2, CYP17, CYP17A1, CYP19, CYP19A1, CYP1A2, CYP1B1, CYP21A2, CYP27A1, CYP27B1, CYP2A6, CYP2C, CYP2C19, CYP2C9, CYP2D, CYP2D6, CYP2D7P1, CYP3A4, CYP7B1, CYPB1, CYP11B1, CYP1A1, CYP1B1, CYRAA, D40, DAD1, DAM, DAM-10/MAGE-B1, DAM-6/MAGE-B2, DAX1, DAZ, DBA, DBH, DBI, DBT, DCC, DC-CK1, DCK, DCR, DCX, DDB1, DDB2, DDIT3, DDU, DECR1, DEK-CAN, DEM, DES, DF, DFN2, DFN4, DFN6, DFNA4, DFNA5, DFNB5, DGCR, DHCR7, DHFR, DHOF, DHS, DIA1, DIAPH2, DIAPH1, DIH1, DIO1, DISC1, DKC1, DLAT, DLD, DLL3, DLX3, DMBT1, DMD, DM1, DMPK, DMWD, DNAI1, DNASE1, DNMT3B, DPEP1, DPYD, DPYS, DRD2, DRD4, DRPLA, DSCR1, DSG1, DSP, DSPP, DSS, DTDP2, DTR, DURS1, DWS, DYS, DYSF, DYT2, DYT3, DYT4, DYT2, DYT1, DYX1, EBAF, EBM, EBNA, EBP, EBR3, EBS1, ECA1, ECB2, ECE1, ECGF1, ECT, ED2, ED4, EDA, EDAR, ECA1, EDN3, EDNRB, EEC1, EEF1A1L14, EEGV1, EFEMP1, EFTUD2/m, EGFR, EGFR/Her1, EGI, EGR2, EIF2AK3, eIF4G, EKV, E1 IS, ELA2, ELF2, ELF2M, ELK1, ELN, ELONG, EMD, EML1, EMMPRIN, EMX2, ENA-78, ENAM, END3, ENG, ENO1, ENPP1, ENUR2, ENUR1, EOS, EP300, EPB41, EPB42, EPCAM, EPD, EphA1, EphA2, EphA3, EphrinA2, EphrinA3, EPHX1, EPM2A, EPO, EPOR, EPX, ERBB2, ERCC2 ERCC3, ERCC4, ERCC5, ERCC6, ERVR, ESR1, ETFA, ETFB, ETFDH, ETM1, ETV6-AML1, ETV1, EVC, EVR2, EVR1, EWSR1, EXT2, EXT3, EXT1, EYA1, EYCL2, EYCL3, EYCL1, EZH2, F10, F11, F12, F13A1, F13B, F2, F5, F5F8D, F7, F8, F8C, F9, FABP2, FACL6, FAH, FANCA, FANCB, FANCC, FANCD2, FANCF, FasL, FBN2, FBN1, FBP1, FCG3RA, FCGR2A, FCGR2B, FCGR3A, FCHL, FCMD, FCP1, FDPSL5, FECH, FEO, FEOM1, FES, FGA, FGB, FGD1, FGF2, FGF23, FGF5, FGFR2, FGFR3, FGFR1, FGG, FGS1, FH, FIC1, FIH, F2, FKBP6, FLNA, FLT4, FMO3, FMO4, FMR2, FMR1, FN, FN1/m, FOXC1, FOXE1, FOXL2, FOXO1A, FPDMM, FPF, Fra-1, FRAXF, FRDA, FSHB, FSHMD1A, FSHR, FTH1, FTHL17, FTL, FTZF1, FUCA1, FUT2, FUT6, FUT1, FY, G250, G250/CAIX, G6PC, G6PD, G6PT1, G6PT2, GAA, GABRA3, GAGE-1, GAGE-2, GAGE-3, GAGE-4, GAGE-5, GAGE-6, GAGE-7b, GAGE-8, GALC, GALE, GALK1, GALNS, GALT, GAMT, GAN, GAST, GASTRIN17, GATA3, GATA, GBA, GBE, GC, GCDH, GCGR, GCH1, GCK, GCP-2, GCS1, G-CSF, GCSH, GCSL, GCY, GDEP, GDF5, GDI1, GDNF, GDXY, GFAP, GFND, GGCX, GGT1, GH2, GH1, GHR, GHRHR, GHS, GIF, GINGF, GIP, GJA3, GJA8, GJB2, GJB3, GJB6, GJB1, GK, GLA, GLB, GLB1, GLC3B, GLC1B, GLC1C, GLDC, GLI3, GLP1, GLRA1, GLUD1, GM1 (fuc-GM1), GM2A, GM-CSF, GMPR, GNAI2, GNAS, GNAT1, GNB3, GNE, GNPTA, GNRH, GNRH1, GNRHR, GNS, GnT-V, gp100, GP1BA, GP1BB, GP9, GPC3, GPD2, GPDS1, GP1, GP1BA, GPN1LW, GPNMB/m, GPSC, GPX1, GRHPR, GRK1, GROα, GROβ, GROγ, GRPR, GSE, GSM1, GSN, GSR, GSS, GTD, GTS, GUCA1A, GUCY2D, GULOP, GUSB, GUSM, GUST, GYPA, GYPC, GYS1, GYS2, H0KPP2, H0MG2, HADHA, HADHB, HAGE, HAGH, HAL, HAST-2, HB 1, HBA2, HBA1, HBB, HBBP1, HBD, HBE1, HBG2, HBG1, HBHR, HBP1, HBQ1, HBZ, HBZP, HCA, HCC-1, HCC-4, HCF2, HCG, HCL2, HCL1, HCR, HCVS, HD, HPN, HER2, HER2/NEU, HER3, HERV-K-MEL, HESX1, HEXA, HEXB, HF1, HFE, HF1, HGD, HHC2, HHC3, HHG, HK1 HLA-A, HLA-A*0201-R170I, HLA-A11/m, HLA-A2/m, HLA-DPB1 HLA-DRA, HLCS, HLXB9, HMBS, HMGA2, HMGCL, HMI, HMN2, HMOX1, HMS1 HMW-MAA, HND, HNE, HNF4A, HOAC, HOMEOBOX NKX 3.1, HOM-TES-14/SCP-1, HOM-TES-85, HOXA1 HOXD13, HP, HPC1, HPD, HPE2, HPE1, HPFH, HPFH2, HPRT1, HPS1, HPT, HPV-E6, HPV-E7, HR, HRAS, HRD, HRG, HRPT2, HRPT1, HRX, HSD11B2, HSD17B3, HSD17B4, HSD3B2, HSD3B3, HSN1, HSP70-2M, HSPG2, HST-2, HTC2, HTC1, hTERT, HTN3, HTR2C, HVBS6, HVBS1, HVEC, HV1S, HYAL1, HYR, I-309, IAB, IBGC1, IBM2, ICAM1, ICAM3, iCE, ICHQ, ICR5, ICR1, ICS 1, IDDM2, IDDM1, IDS, IDUA, IF, IFNa/b, IFNGR1, IGAD1, IGER, IGF-1R, IGF2R, IGF1, IGH, IGHC, IGHG2, IGHG1, IGHM, IGHR, IGKC, IHG1, IHH, IKBKG, IL1, IL-1 RA, IL10, IL-11, IL12, IL12RB1, IL13, IL-13Rα2, IL-15, IL-16, IL-17, IL18, IL-1a, IL-1α, IL-1b, IL-1β, IL1RAPL1, IL2, IL24, IL-2R, IL2RA, IL2RG, IL3, IL3RA, IL4, IL4R, IL4R, IL-5, IL6, IL-7, IL7R, IL-8, IL-9, Immature laminin receptor, IMMP2L, INDX, INFGR1, INFGR2, INFα, IFN [symbol] INFγ, INS, INSR, INVS, IP-10, IP2, IPF1, IP1, IRF6, IRS1, ISCW, ITGA2, ITGA2B, ITGA6, ITGA7, ITGB2, ITGB3, ITGB4, ITIH1, ITM2B, IV, IVD, JAG1, JAK3, JBS, JBTS1, JMS, JPD, KAL1, KAL2, KAL1, KLK2, KLK4, KCNA1, KCNE2, KCNE1, KCNH2, KCNJ1, KCNJ2, KCNJ1, KCNQ2, KCNQ3, KCNQ4, KCNQ1, KCS, KERA, KFM, KFS, KFSD, KHK, ki-67, KIAA0020, KIAA0205, KIAA0205/m, KIF1B, KIT, KK-LC-1, KLK3, KLKB1, KM-HN-1, KMS, KNG, KNO, K-RAS/m, KRAS2, KREV1, KRT1, KRT10, KRT12, KRT13, KRT14, KRT14L1, KRT14L2, KRT14L3, KRT16, KRT16L1, KRT16L2, KRT17, KRT18, KRT2A, KRT3, KRT4, KRT5, KRT6 A, KRT6B, KRT9, KRTHB1, KRTHB6, KRT1, KSA, KSS, KWE, KYNU, L0H19CR1, L1CAM, LAGE, LAGE-1, LALL, LAMA2, LAMA3, LAMB3, LAMB1, LAMC2, LAMP2, LAP, LCA5, LCAT, LCCS, LCCS 1, LCFS2, LCS1, LCT, LDHA, LDHB, LDHC, LDLR, LDLR/FUT, LEP, LEWISY, LGCR, LGGF-PBP, LGI1, LGMD2H, LGMD1A, LGMDIB, LHB, LHCGR, LHON, LHRH, LHX3, LIF, LIG1, LIMM, LIMP2, LIPA, LIPA, LIPB, LIPC, LIVIN, LICAM, LMAN1, LMNA, LMX1B, LOLR, LOR, LOX, LPA, LPL, LPP, LQT4, LRP5, LRS 1, LSFC, LT-β, LTBP2, LTC4S, LYL1, XCL1, LYZ, M344, MA50, MAA, MADH4, MAFD2, MAFD1, MAGE, MAGE-A1, MAGE-A10, MAGE-A12, MAGE-A2, MAGE-A3, MAGE-A4, MAGE-A6, MAGE-A9, MAGEB1, MAGE-B10, MAGE-B16, MAGE-B17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2, MGB1, MGB2, MAN2A1, MAN2B1, MANBA, MANBB, MAOA, MAOB, MAPK8IP1, MAPT, MART-1, MART-2, MART2/m, MAT1A, MBL2, MBP, MBS1, MC1R, MC2R, MC4R, MCC, MCCC2, MCCC1, MCDR1, MCF2, MCKD, MCL1, MC1R, MCOLN1, MCOP, MCOR, MCP-1, MCP-2, MCP-3, MCP-4, MCPH2, MCPH1, MCS, M-CSF, MDB, MDCR, MDM2, MDRV, MDS 1, ME1, ME1/m, ME2, ME20, ME3, MEAX, MEB, MEC CCL-28, MECP2, MEFV, MELANA, MELAS, MEN1 MSLN, MET, MF4, MG50, MG50/PXDN, MGAT2, MGAT5, MGC1 MGCR, MGCT, MG1, MGP, MHC2TA, MHS2, MHS4, MIC2, MIC5, MIDI, MIF, MIP, MIP-5/HCC-2, MITF, MJD, MKI67, MKKS, MKS1, MLH1, MLL, MLLT2, MLLT3, MLLT7, MLLT1, MLS, MLYCD, MMA1a, MMP 11, MMVP1, MN/CA IX-Antigen, MNG1, MN1, MOC31, MOCS2, MOCS1, MOG, MORC, MOS, MOV18, MPD1, MPE, MPFD, MPI, MPIF-1, MPL, MPO, MPS3C, MPZ, MRE11A, MROS, MRP1, MRP2, MRP3, MRSD, MRX14, MRX2, MRX20, MRX3, MRX40, MRXA, MRX1, MS, MS4A2, MSD, MSH2, MSH3, MSH6, MSS, MSSE, MSX2, MSX1, MTATP6, MTC03, MTCO1, MTCYB, MTHFR, MTM1, MTMR2, MTND2, MTND4, MTND5, MTND6, MTND1, MTP, MTR, MTRNR2, MTRNR1, MTRR, MTTE, MTTG, MTTI, MTTK, MTTL2, MTTL1, MTTN, MTTP, MTTS1, MUC1, MUC2, MUC4, MUC5AC, MUM-1, MUM-1/m, MUM-2, MUM-2/m, MUM-3, MUM-3/m, MUT, mutant p21 ras, MUTYH, MVK, MX2, MXI1, MY05A, MYB, MYBPC3, MYC, MYCL2, MYH6, MYH7, MYL2, MYL3, MYMY, MYO15A, MYO1G, MYOSA, MYO7A, MYOC, Myosin/m, MYP2, MYP1, NA88-A, N-acetylglucosaminyltransferase-V, NAGA, NAGLU, NAMSD, NAPB, NAT2, NAT, NBIA1, NBS1, NCAM, NCF2, NCF1, NDN, NDP, NDUFS4, NDUFS7, NDUFS8, NDUFV1, NDUFV2, NEB, NEFH, NEM1, Neo-PAP, neo-PAP/m, NEU1, NEUROD1, NF2, NF1, NFYC/m, NGEP, NHS, NKS1, NKX2E, NM, NME1, NMP22, NMTC, NODAL, NOG, NOS3, NOTCH3, NOTCH1, NP, NPC2, NPC1, NPHL2, NPHP1, NPHS2, NPHS1, NPM/ALK, NPPA, NQO1, NR2E3, NR3C1, NR3C2, NRAS, NRAS/m, NRL, NROB1, NRTN, NSE, NSX, NTRK1, NUMA1, NXF2, NY-CO1, NY-ESO1, NY-ESO-B, NY-LU-12, ALDOA, NYS2, NYS4, NY-SAR-35, NYS1, NYX, OA3, OA1, OAP, OASD, OAT, OCA1, OCA2, OCD1, OCRL, OCRL1, OCT, ODDD, ODT1, OFC1, OFD1, OGDH, OGT, OGT/m, OPA2, OPA1, OPD1, OPEM, OPG, OPN, OPN1LW, OPN1MW, OPN1SW, OPPG, OPTB1, TTD, ORM1, ORP1, OS-9, OS-9/m, OSM LIF, OTC, OTOF, OTSC1, OXCT1, OYTES1, P15, P190 MINOR BCR-ABL, P2RY12, P3, P16, P40, P4HB, P-501, P53, P53/m, P97, PABPN1, PAFAH1B1, PAFAH1P1, PAGE-4, PAGE-5, PAH, PAI-1, PAI-2, PAK3, PAP, PAPPA, PARK2, PART-1, PATE, PAX2, PAX3, PAX6, PAX7, PAX8, PAX9, PBCA, PBCRA1, PBT, PBX1, PBXP1, PC, PCBD, PCCA, PCCB, PCK2, PCK1, PCLD, PCOS1, PCSK1, PDB1, PDCN, PDE6A, PDE6B, PDEF, PDGFB, PDGFR, PDGFRL, PDHA1, PDR, PDX1, PECAM1, PEE1, PEO1, PEPD, PEX10, PEX12, PEX13, PEX3, PEX5, PEX6, PEX7, PEX1, PF4, PFBI, PFC, PFKFB1, PFKM, PGAM2, PGD, PGK1, PGK1P1, PGL2, PGR, PGS, PHA2A, PHB, PHEX, PHGDH, PHKA2, PHKA1, PHKB, PHKG2, PHP, PHYH, PI, PI3, PIGA, PIM1-KINASE, PIN1, PIP5K1B, PITX2, PITX3, PKD2, PKD3, PKD1, PKDTS, PKHD1, PKLR, PKP1, PKU1, PLA2G2A, PLA2G7, PLAT, PLEC1, PLG, PLI, PLOD, PLP1, PMEL17, PML, PML/RARα, PMM2, PMP22, PMS2, PMS1, PNKD, PNLIP, POF1, POLA, POLH, POMC, PON2, PON1, PORC, POTE, POU1F1, POU3F4, POU4F3, POU1F1, PPAC, PPARG, PPCD, PPGB, PPH1, PPKB, PPMX, PPOX, PPP1R3A, PPP2R2B, PPT1, PRAME, PRB, PRB3, PRCA1, PRCC, PRD, PRDX5/m, PRF1, PRG4, PRKAR1A, PRKCA, PRKDC, PRKWNK4, PRNP, PROC, PRODH, PROM1, PROP1, PROS1, PRST, PRP8, PRPF31, PRPF8, PRPH2, PRPS2, PRPS1, PRS, PRSS7, PRSS1, PRTN3, PRX, PSA, PSAP, PSCA, PSEN2, PSEN1, PSG1, PSGR, PSM, PSMA, PSORS1, PTC, PTCH, PTCH1, PTCH2, PTEN, PTGS1, PTH, PTHR1, PTLAH, PTOS1, PTPN12, PTPNI1, PTPRK, PTPRK/m, PTS, PUJO, PVR, PVRL1, PWCR, PXE, PXMP3, PXR1, PYGL, PYGM, QDPR, RAB27A, RAD54B, RAD54L, RAG2, RAGE, RAGE-1, RAG1, RAP1, RARA, RASA1, RBAF600/m, RB1, RBP4, RBP4, RBS, RCA1, RCAS1, RCCP2, RCD1, RCV1, RDH5, RDPA, RDS, RECQL2, RECQL3, RECQL4, REG1A, REHOBE, REN, RENBP, RENS1, RET, RFX5, RFXANK, RFXAP, RGR, RHAG, RHAMM/CD168, RHD, RHO, Rip-1, RLBP1, RLN2, RLN1, RLS, RMD1, RMRP, ROM1, ROR2, RP, RP1, RP14, RP17, RP2, RP6, RP9, RPD1, RPE65, RPGR, RPGRIP1, RP1, RP10, RPS19, RPS2, RPS4X, RPS4Y, RPS6KA3, RRAS2, RS1, RSN, RSS, RU1, RU2, RUNX2, RUNX1, RS, RTR1, S-100, SAA1, SACS, SAG, SAGE, SALL1, SARDH, SART1, SART2, SART3, SAS, SAX1, SCA2, SCA4, SCA5, SCA7, SCA8, SCA1, SCC, SCCD, SCF, SCLC1, SCN1A, SCN1B, SCN4A, SCN5A, SCNN1A, SCNN1B, SCNN1G, SCO2, SCP1, SCZD2, SCZD3, SCZD4, SCZD6, SCZD1, SDF-1 [symbol] / [symbol] SDHA, SDHD, SDYS, SEDL, SERPENA7, SERPINA3, SERPINA6, SERPINA1, SERPINC1, SERPIND1, SERPINE1, SERPINF2, SERPING1, SERPINI1, SFTPA1, SFTPB, SFTPC, SFTPD, SGCA, SGCB, SGCD, SGCE, SGM1, SGSH, SGY-1, SH2D1A, SHBG, SHFM2, SHFM3, SHFM1, SHH, SHOX, SI, SIAL, SIALYL LEWISX, SIASD, S11, SIM1, SIRT2/m, SIX3, SJS1, SKP2, SLC10A2, SLC12A1, SLC12A3, SLC17A5, SLC19A2, SLC22A1L, SLC22A5, SLC25A13, SLC25A15, SLC25A20, SLC25A4, SLC25A5, SLC25A6, SLC26A2, SLC26A3, SLC26A4, SLC2A1, SLC2A2, SLC2A4, SLC3A1, SLC4A1, SLC4A4, SLC5A1, SLC5A5, SLC6A2, SLC6A3, SLC6A4, SLC7A7, SLC7A9, SLC11A1, SLOS, SMA, SMAD1, SMAL, SMARCB1, SMAX2, SMCR, SMCY, SM1, SMN2, SMN1, SMPD1, SNCA, SNRPN, SOD2, SOD3, SOD1, SOS1, SOST, SOX9, SOX10, Sp17, SPANXC, SPG23, SPG3A, SPG4, SPG5A, SPG5B, SPG6, SPG7, SPINK1, SPINK5, SPPK, SPPM, SPSMA, SPTA1, SPTB, SPTLC1, SRC, SRD5A2, SRPX, SRS, SRY, βhCG, SSTR2, SSX1, SSX2 (HOM-MEL-40/SSX2), SSX4, ST8, STAMP-1, STAR, STARP1, STATH, STEAP, STK2, STK11, STn/KLH, STO, STOM, STS, SUOX, SURF1, SURVIVIN-2B, SYCP1, SYM1, SYN1, SYNS1, SYP, SYT/SSX, SYT-SSX-1, SYT-SSX-2, TA-90, TAAL6, TACSTD1, TACSTD2, TAG72, TAF7L, TAF1, TAGE, TAG-72, TALI, TAM, TAP2, TAP1, TAPVR1, TARC, TARP, TAT, TAZ, TBP, TBX22, TBX3, TBX5, TBXA2R, TBXAS1, TCAP, TCF2, TCF1, TCIRG1, TCL2, TCL4, TCL1A, TCN2, TCOF1, TCR, TCRA, TDD, TDFA, TDRD1, TECK, TECTA, TEK, TEL/AML1, TELAB1, TEX15, TF, TFAP2B, TFE3, TFR2, TG, TGFA, TGF-β, TGFBI, TGFB1, TGFBR2, TGFBRE, TGFβ, TGFβRII, TGIF, TGM-4, TGM1, TH, THAS, THBD, THC, THC2, THM, THPO, THRA, THRB, TIMM8A, TIMP2, TIMP3, TIMP1, TITF1, TKCR, TKT, TLP, TLR1, TLR10, TLR2, TLR3, TLR4, TLR4, TLR5, TLR6, TLR7, TLR8, TLR9, TLX1, TM4SF1, TM4SF2, TMC1, TMD, TMIP, TNDM, TNF, TNFRSF11A, TNFRSF1A, TNFRSF6, TNFSF5, TNFSF6, TNFα, TNFβ, TNNI3, TNNT2, TOC, TOP2A, TOP1, TP53, TP63, TPA, TPBG, TPI, TPI/m, TPI1, TPM3, TPM1, TPMT, TPO, TPS, TPTA, TRA, TRAG3, TRAPPC2, TRC8, TREH, TRG, TRH, TRIM32, TRIM37, TRP1, TRP2, TRP-2/6b, TRP-2/INT2, Trp-p8, TRPS1, TS, TSC2, TSC3, TSC1, TSG101, TSHB, TSHR, TSP-180, TST, TTGA2B, TTN, TTPA, TTR, TU M2-PK, TULP1, TWIST, TYH, TYR, TYROBP, TYROBP, TYRP1, TYS, UBE2A, UBE3A, UBE1, UCHL1, UFS, UGT1A, ULR, UMPK, UMPS, UOX, UPA, UQCRC1, URO5, UROD, UPK1B, UROS, USH2A, USH3A, USH1A, USH1C, USP9Y, UV24, VBCH, VCF, VDI, VDR, VEGF, VEGFR-2, VEGFR-1, VEGFR-2/FLK-1, VHL, VIM, VMD2, VMD1, VMGLOM, VNEZ, VNF, VP, VRNI, VWF, VWS, WAS, WBS2, WFS2, WFS1, WHCR, WHN, WISP3, WMS, WRN, WS2A, WS2B, WSN, WSS, WT2, WT3, WT1, WTS, WWS, XAGE, XDH, XIC, XIST, XK, XM, XPA, XPC, XRCC9, XS, ZAP70, ZFHX1B, ZFX, ZFY, ZIC2, ZIC3, ZNF145, ZNF261, ZNF35, ZNF41, ZNF6, ZNF198, ZWS1. The base-modified RNA of the invention may also contain two or more coding regions for the above proteins. Accordingly, the inventive RNA may e.g. be bi- or multicistronic.

Preferably, the protein encoded by the inventive RNA is selected (without implying any limitation) from e.g. growth hormones or growth factors, for example for promoting growth in a (transgenic) living being, such as, for example, TGFα and the IGFs (insulin-like growth factors), proteins that influence the metabolism and/or haematopoiesis, such as, for example, α-anti-trypsin, LDL receptor, erythropoietin (EPO), insulin, GATA-1, etc., or proteins such as, for example, factors VIII and XI of the blood coagulation system, etc. Such proteins further include enzymes, such as, for example, β-galactosidase (lacZ), DNA restriction enzymes (e.g. EcoRI, HindIII, etc.), lysozymes, etc., or proteases, such as, for example, papain, bromelain, keratinases, trypsin, chymotrypsin, pepsin, renin (chymosin), suizyme, nortase, etc. These proteins may be provided by the inventive base-modified RNA, which is characterized by an increased level of expression. Accordingly, the invention provides a technology which allows to substitute proteins which are defective in the organism to be treated (e.g. either due to mutations, due to defective or missing expression). Accordingly, the invention allows to provide effective and increased expression of proteins, which are not functional in the organism to be treated, as e.g. occurring in monogenetic disorders.

Alternatively, the present invention may also provide therapeutic proteins, e.g. antibodies or proteases etc. which allow to cure a specific disease due to e.g. (over)expression of a dysfunctional or exogenous proteins causing disorders or diseases. Accordingly, the invention may be used to therapeutically introduce the inventive RNA into the organism, which attacks a pathogenic organism (virus, bacteria etc). E.g. RNA encoding therapeutic proteases may be used to cleave viral proteins which are essential to the viral assembly or other essential steps of virus production.

Alternatively, the proteins coded for by the base-modified RNA used according to the invention may be used to stimulate an adaptive immune response by providing efficiently expressed antigens which elicit an adaptive immune response, whereas the underlying base-modified RNA does not provoke any immune reaction as such. Insofar, the invention may allow to provide vaccines based on the base-modified RNA, which expresses increased levels of the antigenic protein or peptide. These vaccines may be used for the provision of tumour vaccines providing tumour antigens or antigens derived from pathogenic microorganisms causing e.g. infectious diseases. Specifically preferred proteins coded for by the base-modified RNA used according to the invention can be selected from the following antigens: tumour-specific surface antigens (TSSAs), for example 5T4, α5β1-integrin, 707-AP, AFP, ART-4, B7H4, BAGE, β-catenin/m, Bcr-abl, MN/C IX antigen, CA125, CAMEL, CAP-1, CASP-8, β-catenin/m, CD4, CD19, CD20, CD22, CD25, CDC27/m, CD 30, CD33, CD52, CD56, CD80, CDK4/m, CEA, CT, Cyp-B, DAM, EGFR, ErbB3, ELF2M, EMMPRIN, EpCam, ETV6-AML1, G250, GAGE, GnT-V, Gp100, HAGE, HER-2/new, HLA-A*0201-R170I, HPV-E7, HSP70-2M, HAST-2, hTERT (or hTRT), iCE, IGF-1R, IL-2R, IL-5, KIAA0205, LAGE, LDLR/FUT, MAGE, MART-1/melan-A, MART-2/Ski, MC1R, myosin/m, MUC1, MUM-1, -2, -3, NA88-A, PAP, NY-ESO1, proteinase-3, p190 minor bcr-abl, Pml/RARα, PRAME, PSA, PSM, PSMA, RAGE, RU1 or RU2, SAGE, SART-1 or SART-3, survivin, TEL/AML1, TGFβ, TPI/m, TRP-1, TRP-2, TRP-2/INT2, VEGF and WT1, or from sequences such as, for example, NY-Eso-1 or NY-Eso-B.

Another class of proteins, which may be expressed by the inventive base-modified RNA may include proteins which modulate various intracellular pathways by e.g. signal transmission modulation (inhibition or stimulation) which may influence pivotal intracellular processes like apoptosis, cell growth etc, in particular with respect to the organism's immune system. Accordingly, immune modulators, e.g. cytokines, lymphokines, monokines, interferones etc. may be expressed efficiently by the base-modified RNA. Preferably, these proteins therefore also include, for example, cytokines of class I of the cytokine family that contain 4 position-specific conserved cysteine residues (CCCC) and a conserved sequence motif Trp-Ser-X-Trp-Ser (WSXWS), wherein X represents an unconserved amino acid. Cytokines of class I of the cytokine family include the GM-CSF sub-family, for example IL-3, IL-5, GM-CSF, the IL-6 sub-family, for example IL-6, IL-11, IL-12, or the IL-2 sub-family, for example IL-2, IL-4, IL-7, IL-9, IL-15, etc., or the cytokines IL-1α, IL-1β, IL-10 etc. By analogy, such proteins can also include cytokines of class II of the cytokine family (interferon receptor family), which likewise contain 4 position-specific conserved cysteine residues (CCCC) but no conserved sequence motif Trp-Ser-X-Trp-Ser (WSXWS). Cytokines of class II of the cytokine family include, for example, IFN-α, IFN-β, IFN-γ, etc. Proteins coded for by the base-modified RNA used according to the invention can further include also cytokines of the tumour necrosis family, for example TNF-α, TNF-β, TNF-RI, TNF-RII, CD40, Fas, etc., or cytokines of the chemokine family, which contain 7 transmembrane helices and interact with G-protein, for example IL-8, MIP-1, RANTES, CCR5, CXR4, etc. Such proteins can also be selected from apoptosis factors or apoptosis-related or -linked proteins, including AIF, Apaf, for example Apaf-1, Apaf-2, Apaf-3, or APO-2 (L), APO-3 (L), apopain, Bad, Bak, Bax, Bcl-2, Bcl-x_{L}, Bcl-x_{S}, bik, CAD, calpain, caspases, for example caspase-1, caspase-2, caspase-3, caspase-4, caspase-5, caspase-6, caspase-7, caspase-8, caspase-9, caspase-10, caspase-11, ced-3, ced-9, c-Jun, c-Myc, crm A, cytochrome C, CdR1, DcR1, DD, DED, DISC, DNA-PK_{CS}, DR3, DR4, DR5, FADD/MORT-1, FAK, Fas (Fas ligand CD95/fas (receptor)), FLICE/MACH, FLIP, fodrin, fos, G-actin, Gas-2, gelsolin, granzymes A/B, ICAD, ICE, JNK, lamin A/B, MAP, MCL-1, Mdm-2, MEKK-1, MORT-1, NEDD, NF-_{κ}B, NuMa, p53, PAK-2, PARP, perforin, PITSLRE, PKCδ, pRb, presenilin, prICE, RAIDD, Ras, RIP, sphingomyelinase, thymidine kinase from Herpes simplex, TRADD, TRAF2, TRAIL, TRAIL-R1, TRAIL-R2, TRAIL-R3, transglutaminase, etc.

Finally, the base-modified RNA may also code for antigen specific T cell receptors. The T cell receptor or TCR is a molecule found on the surface of T lymphocytes (or T cells) that is generally responsible for recognizing antigens bound to major histocompatibility complex (MHC) molecules. It is a heterodimer consisting of an alpha and beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors and specialized accessory molecules. Hence, these proteins allow to specifically target specific antigen and may support the functionality of the immune system due to their targeting properties. Accordingly, transfection of cells in vivo by administering base-modified RNA coding for these receptors or, preferably, an ex vivo cell transfection approach (e.g. by transfecting specifically certain immune cells), may be pursued. The T cell receptor molecules introduced recognize specific antigens on MHC molecule and may thereby support the immune system's awareness of antigens to be attacked.

Proteins that can be coded for by the base-modified RNA used according to the invention further include also those proteins or protein sequences that have a sequence identity of at least 80% or 85%, preferably at least 90%, more preferably at least 95% and most preferably at least 99%, with one of the proteins described above, e.g. their native sequence. The base-modified nucleotides and their native (non base-modified) analog are considered to be “identical” herein.

The term “identity” in the present application means that the sequences are compared with one another, as hereinbelow. In order to determine the percentage identity of two nucleic acid sequences, the sequences can first be arranged relative to one another (alignment) in order to permit subsequent comparison of the sequences. To this end, for example, gaps can be introduced into the sequence of the first nucleic acid sequence and the nucleotides can be compared with the corresponding position of the second nucleic acid sequence. When a position in the first nucleic acid sequence is occupied with the same nucleotide as in a position in the second sequence, then the two sequences are identical at that position. The percentage identity between two sequences is a function of the number of identical positions divided by the number of all compared positions of the studied sequences. If, for example, a specific sequence identity is assumed for a particular nucleic acid (e.g. a nucleic acid coding for a protein as described above) in comparison with a reference nucleic acid (e.g. a nucleic acid of the prior art) having a defined length, then this percentage identity is indicated relatively in relation to the reference nucleic acid. Therefore, starting, for example, from a nucleic acid that has 50% sequence identity with a reference nucleic acid having a length of 100 nucleotides, that nucleic acid can represent a nucleic acid having a length of 50 nucleotides that is wholly identical with a section of the reference nucleic acid having a length of 50 nucleotides. It can, however, also represent a nucleic acid having a length of 100 nucleotides that has 50% identity, that is to say in this case 50% identical nucleic acids, with the reference nucleic acid over its entire length. Alternatively, that nucleic acid can be a nucleic acid having a length of 200 nucleotides that, in a section of the nucleic acid having a length of 100 nucleotides, is wholly identical with the reference nucleic acid having a length of 100 nucleotides. Other nucleic acids naturally fulfil these criteria equally. The comments made regarding the identity of nucleic acids apply equally to proteins or peptide sequences.

The determination of the percentage identity of two sequences can be carried out by means of a mathematical algorithm. A preferred but non-limiting example of a mathematical algorithm which can be used for comparing two sequences is the algorithm of Karlin et al. (1993), PNAS USA, 90:5873-5877. Such an algorithm is integrated into the NBLAST program, with which sequences having a desired identity with the sequences of the present invention can be identified. In order to obtain a gapped alignment as described above, the “Gapped BLAST” program can be used, as described in Altschul et al. (1997), Nucleic Acids Res, 25:3389-3402. When using BLAST and Gapped BLAST programs, the default parameters of the particular program (e.g. NBLAST) can be used. The sequences can further be aligned using version 9 of GAP (global alignment program) from “Genetic Computing Group”, using the default (BLOSUM62) matrix (values −4 to +11) with a gap open penalty of −12 (for the first zero of a gap) and a gap extension penalty of −4 (for each additional successive zero in the gap). After the alignment, the percentage identity is calculated by expressing the number of correspondences as a percentage of the nucleic acids in the claimed sequence. The described methods for determining the percentage identity of two nucleic acid sequences can also be applied correspondingly to amino acid sequences, if required.

According to a preferred embodiment, the base-modified RNA used according to the invention, as well as containing the section coding for the protein, can additionally contain at least one further functional section on the RNA sequence that codes for another therapeutic component. This other therapeutic component may be selected according to the disease to be treated. While this other component may have e.g. immunosuppressive properties when treating e.g. autoimmune diseases (e.g. coding for an immunosuppressant), it may alternatively have immunostimulating properties (enhancing the adaptive immune response elicited by the immunogenic tumour or pathogenic antigen), if the base-modified RNA is used for vaccination purposes (for example for treating infectious or tumour diseases). Accordingly, the immunostimulating component additionally being encoded on the base-modified RNA may be selected, for example from a cytokine (monokine, lymphokine, interleukin or chemokine) that promotes the immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-α, IFN-β, INF-γ, GM-CSF, G-CSF, M-CSF, LT-β or TNF-α, growth factors, such as hGH. This at least one additional component of the base-modified RNA may is typically combined with an IRES thereby forming bi- or multicistronic base-modified RNAs.

In a further preferred embodiment, the base-modified RNA used according to the invention can code for a secretory signal peptide in addition to the protein as described above. Such signal peptides are (signal) sequences that conventionally comprise a length of from 15 to 30 amino acids and are located preferably at the N-terminus of the (poly)peptide that is coded for. Signal peptides typically allow the transport of a protein fused thereto (in this case, for example, a therapeutically active protein) into a defined cellular compartment, preferably the cell surface, the endoplasmic reticulum or the endosomal-lysosomal compartment. Examples of signal sequences which can be used according to the invention are, for example, signal sequences of conventional and non-conventional MHC molecules, cytokines, immunoglobulins, of the invariant chain, Lamp1, tapasin, Erp57, calreticulin and calnexin, and all further membrane-located endosomal-lysosomal- or endoplasmatic-reticulum-associated proteins. Preference is given to the use of the signal peptide of the human MHC class I molecule HLA-A*0201.

According to a particular embodiment, the base-modified RNA used according to the invention can contain a lipid modification. Such a lipid-modified RNA typically consists of a base-modified RNA used according to the invention, as described above, at least one linker covalently linked with that RNA, and at least one lipid covalently linked with the respective linker. Alternatively, the lipid-modified base-modified RNA used according to the invention consists of (at least) one base-modified RNA used according to the invention, as described above, and at least one (bifunctional) lipid covalently linked with that RNA. According to a third alternative, the lipid-modified base-modified RNA used according to the invention consists of a base-modified RNA used according to the invention, as described above, at least one linker linked with that RNA, and at least one lipid linked covalently with the respective linker and at least one (bifunctional) lipid covalently linked (without a linker) with the base-modified RNA used according to the invention.

The lipid used for the lipid modification of the base-modified RNA used according to the invention is typically a lipid or a lipophilic radical that preferably is itself biologically active. Such lipids preferably include natural substances or compounds such as, for example, vitamins, e.g. α-tocopherol (vitamin E), including RRR-α-tocopherol (formerly D-α-tocopherol), L-α-tocopherol, the racemate D,L-α-tocopherol, vitamin E succinate (VES), or vitamin A and its derivatives, e.g. retinoic acid, retinol, vitamin D and its derivatives, e.g. vitamin D and also the ergosterol precursors thereof, vitamin E and its derivatives, vitamin K and its derivatives, e.g. vitamin K and related quinone or phytol compounds, or steroids, such as bile acids, for example cholic acid, deoxycholic acid, dehydrocholic acid, cortisone, digoxygenin, testosterone, cholesterol or thiocholesterol. Further lipids or lipophilic radicals within the scope of the present invention include, without implying any limitation, polyalkylene glycols (Oberhauser et al., Nucl. Acids Res., 1992, 20, 533), aliphatic groups such as, for example, C₁-C₂₀-alkanes, C₁-C₂₀-alkenes or C₁-C₂₀-alkanol compounds, etc., such as, for example, dodecanediol, hexadecanol or undecyl radicals (Saison-Behmoaras et al., EMBO J, 1991, 10, 111; Kabanov et al., FEBS Lett., 1990, 259, 327; Svinarchuk et al., Biochimie, 1993, 75, 49), phospholipids such as, for example, phosphatidylglycerol, diacylphosphatidylglycerol, phosphatidylcholine, dipalmitoylphosphatidylcholine, distearoylphosphatidylcholine, phosphatidylserine, phosphatidylethanolamine, di-hexadecyl-rac-glycerol, sphingolipids, cerebrosides, gangliosides, or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), polyamines or polyalkylene glycols, such as, for example, polyethylene glycol (PEG) (Manoharan et al., Nucleosides & Nucleotides, 1995, 14, 969), hexaethylene glycol (HEG), palmitin or palmityl radicals (Mishra et al., Biochim. Biophys. Acta, 1995, 1264, 229), octadecylamines or hexylaminocarbonyloxycholesterol radicals (Crooke et al., J. Pharmacol. Exp. Ther., 1996, 277, 923), and also waxes, terpenes, alicyclic hydrocarbons, saturated and mono- or poly-unsaturated fatty acid radicals, etc.

Linking between the lipid and the base-modified RNA used according to the invention can in principle take place at any nucleotide, at the base or the sugar radical of any nucleotide, at the 3′ and/or 5′ end, and/or at the phosphate backbone of the base-modified RNA used according to the invention. Particular preference is given according to the invention to a terminal lipid modification of the base-modified RNA at the 3′ and/or 5′ end thereof. A terminal modification has a number of advantages over modifications within the sequence. On the one hand, modifications within the sequence can influence the hybridisation behaviour, which may have an adverse effect in the case of sterically demanding radicals. Modifications within the sequence (sterically demanding modifications) very often also interfere with the translation, which can frequently lead to termination of the protein synthesis. On the other hand, in the case of the synthetic preparation of a lipid-modified base-modified RNA used according to the invention that is modified only terminally, the synthesis of the base-modified RNA can be carried out with commercially available monomers that are obtainable in large quantities, and synthesis protocols known in the prior art can be used.

According to a first preferred embodiment, linking between the base-modified RNA used according to the invention and at least one lipid that is used is effected via a “linker” (covalently linked with the base-modified RNA). Linkers within the scope of the present invention typically have at least two and optionally 3, 4, 5, 6, 7, 8, 9, 10, 10-20, 20-30 or more reactive groups, in each case selected from, for example, a hydroxy group, an amino group, an alkoxy group, etc. One reactive group preferably serves to bind the above-described base-modified RNA used according to the invention. This reactive group can be present in protected form, for example as a DMT group (dimethoxytrityl chloride), as a Fmoc group, as a MMT (monomethoxytrityl) group, as a TFA (trifluoroacetic acid) group, etc. Furthermore, sulfur groups can be protected by disulfides, for example alkylthiols such as, for example, 3-thiopropanol, or by activated components such as 2-thiopyridine. One or more further reactive groups serve according to the invention for the covalent binding of one or more lipids. According to the first embodiment, therefore, a base-modified RNA used according to the invention can bind via the covalently bound linker preferably at least one lipid, for example 1, 2, 3, 4, 5, 5-10, 10-20, 20-30 or more lipid(s), particularly preferably at least 3-8 or more lipid(s) per base-modified RNA. The bound lipids can thereby be bound separately from one another at different positions of the base-modified RNA used according to the invention, or they can be present in the form of a complex at one or more positions of the base-modified RNA. An additional reactive group of the linker can be used for direct or indirect (cleavable) binding to a carrier material, for example a solid phase. Preferred linkers according to the present invention are, for example, glycol, glycerol and glycerol derivatives, 2-aminobutyl-1,3-propanediol and 2-aminobutyl-1,3-propanediol derivatives/skeleton, pyrrolidine linkers or pyrrolidine-containing organic molecules (in particular for a modification at the 3′ end), etc. Glycerol or glycerol derivatives (C₃ anchor) or a 2-aminobutyl-1,3-propanediol derivative/skeleton (C₇ anchor) are particularly preferably used according to the invention as linkers. A glycerol derivative (C₃ anchor) as linker is particularly preferred when the lipid modification can be introduced via an ether bond. If the lipid modification is to be introduced via an amide or a urethane bond, for example, a 2-aminobutyl-1,3-propanediol skeleton (C₇ anchor), for example, is preferred. In this connection, the nature of the bond formed between the linker and the base-modified RNA used according to the invention is preferably such that it is compatible with the conditions and chemicals of amidite chemistry, that is to say it is preferably neither acid- nor base-labile. Preference is given in particular to bonds that are readily obtainable synthetically and are not hydrolysed by the ammoniacal cleavage procedure of a nucleic acid synthesis process. Suitable bonds are in principle all correspondingly suitable bonds, preferably ester bonds, amide bonds, urethane and ether bonds. In addition to the good accessibility of the starting materials (few synthesis steps), particular preference is given to the ether bond owing to its relatively high biological stability towards enzymatic hydrolysis.

According to a second preferred embodiment, the (at least one) base-modified RNA used according to the invention is linked directly with at least one (bifunctional) lipid as described above, that is to say without the use of a linker as described above. In this case, the (bifunctional) lipid used according to the invention preferably contains at least two reactive groups or optionally 3, 4, 5, 6, 7, 8, 9, 10 or more reactive groups, a first reactive group serving to bind the lipid directly or indirectly to a carrier material described herein and at least one further reactive group serving to bind the base-modified RNA. According to the second embodiment, a base-modified RNA used according to the invention can therefore preferably bind at least one lipid (directly without a linker), for example 1, 2, 3, 4, 5, 5-10, 10-20, 20-30 or more lipid(s), particularly preferably at least 3-8 or more lipid(s) per base-modified RNA. The bound lipids can be bound separately from one another at different positions of the base-modified RNA, or they can be present in the form of a complex at one or more positions of the base-modified RNA. Alternatively, at least one base-modified RNA used according to the invention, for example optionally 3, 4, 5, 6, 7, 8, 9, 10, 10-20, 20-30 or more base-modified RNAs, can be bound according to the second embodiment to a lipid as described above via its reactive groups. Lipids that can be used for this second embodiment particularly preferably include those (bifunctional) lipids that permit coupling (preferably at their termini or optionally intramolecularly), such as, for example, polyethylene glycol (PEG) and derivatives thereof, hexaethylene glycol (HEG) and derivatives thereof, alkanediols, aminoalkane, thioalkanols, etc. The nature of the bond between a (bifunctional) lipid and a base-modified RNA as described above is preferably as described for the first preferred embodiment.

According to a third embodiment, linking between the base-modified RNA used according to the invention and at least one lipid as described above can take place via both of the above-mentioned embodiments simultaneously. For example, the base-modified RNA used according to the invention can be linked at one position of the RNA with at least one lipid via a linker (analogously to the first embodiment) and at a different position of the base-modified RNA directly with at least one lipid without the use of a linker (analogously to the second embodiment). For example, at the 3′ end of the base-modified RNA, at least one lipid as described above can be covalently linked with the RNA via a linker, and at the 5′ end of the base-modified RNA, a lipid as described above can be covalently linked with the RNA without a linker. Alternatively, at the 5′ end of a base-modified RNA used according to the invention, at least one lipid as described above can be covalently linked with the base-modified RNA via a linker, and at the 3′ end of the base-modified RNA, a lipid as described above can be covalently linked with the base-modified RNA without a linker. Likewise, covalent linking can take place not only at the termini of the base-modified RNA used according to the invention but also intramolecularly, as described above, for example at the 3′ end and intramolecularly, at the 5′ end and intramolecularly, at the 3′ and 5′ end and intramolecularly, only intramolecularly, etc.

The above-described base-modified RNA used according to the invention can be prepared by preparation processes known in the prior art, for example automatically or manually via known synthetic nucleic acid syntheses (see e.g. Maniatis et al. (2001) supra).

According to a further object of the present invention, the base-modified RNA used according to the invention can be used for the preparation of a pharmaceutical composition for the treatment of tumours and cancer diseases, heart and circulatory diseases, infectious diseases or autoimmune diseases, as well as for the treatment of monogenetic diseases, for example in gene therapy.

A pharmaceutical composition within the scope of the present invention contains a base-modified RNA as described above and optionally a pharmaceutically acceptable carrier and/or further auxiliary substances and additives and/or adjuvants. The pharmaceutical composition used according to the present invention typically comprises a safe and effective amount of a base-modified RNA as described above. As used here, “safe and effective amount” means an amount of the base-modified RNA used according to the invention that is sufficient to significantly induce a positive change in a condition to be treated, for example a tumour or cancer disease, a heart or circulatory disease or an infectious disease, as described hereinbelow. At the same time, however, a “safe and effective amount” is small enough to avoid serious side-effects in the therapy of these diseases, that is to say to permit a sensible relationship between advantage and risk. The determination of these limits typically lies within the scope of sensible medical judgment. The concentration of the base-modified RNA used according to the invention in such pharmaceutical compositions can therefore vary, for example, without implying any limitation, within a wide range of, for example, from 0.1 μg to 100 mg/ml. Such a “safe and effective amount” of a base-modified RNA used according to the invention can vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The pharmaceutical composition described here can be used for human and also for veterinary medical purposes.

If it is required to increase the immunogenicity of the pharmaceutical composition (due to its use for the treatment of e.g. tumours or infectious diseases as a vaccine), the composition can additionally contain one or more auxiliary substances. A synergistic action of the base-modified RNA used according to the invention and of an auxiliary substance optionally additionally contained in the pharmaceutical composition is preferably achieved thereby. Depending on the various types of auxiliary substances, various mechanisms can come into consideration in this respect. For example, compounds that permit the maturation of dendritic cells (DCs), for example lipopolysaccharides, TNF-α or CD40 ligand, form a first class of suitable auxiliary substances. In general, it is possible to use as auxiliary substance any agent that influences the immune system in the manner of a “danger signal” (LPS, GP96, etc.) or cytokines, such as GM-CSF, which allow an immune response produced by the base-modified RNA used according to the invention to be enhanced and/or influenced in a targeted manner and/or an immune reaction to be initiated at the same time. Particularly preferred auxiliary substances are cytokines, such as monokines, lymphokines, interleukins or chemokines, for example IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-α, IFN-β, INF-γ, GM-CSF, G-CSF, M-CSF, LT-β or TNF-α, or interferons, for example IFN-γ, or growth factors, for example hGH.

The above-described pharmaceutical composition if provided as a vaccine to treat tumours or infectious diseases can further additionally contain an adjuvant known in the prior art. In connection with the present invention, adjuvants known in the prior art include, without implying any limitation, stabilising cationic peptides or polypeptides as described above, such as protamine, nucleoline, spermine or spermidine, and cationic polysaccharides, in particular chitosan, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium oxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyldodecanoyl-amide hydroacetate); CALCITRIOL™ (1α,25-dihydroxy-vitamin D3); calcium phosphate gel; CAPTM (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-γ; interleukin-1β; interleukin-2; interleukin-7; interleukin-12; ISCOMS™ (“Immune Stimulating Complexes”); ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MF59™; (squalene-water emulsion); MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH₃); MURAPALMITINE™ and D-MURAPALMITINE™ (Nac-Mur-L-Thr-D-isoGIn-sn-glyceroldipalmitoyl); NAGO (neuraminidase-galactose oxidase); nanospheres or nanoparticles of any composition; NISVs (non-ionic surfactant vesicles); PLEURAN™ (β-glucan); PLGA, PGA and PLA (homo- and co-polymers of lactic acid and glycolic acid; micro-/nano-spheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON™ (QS-21); Quil-A (Quil-A saponin); S-28463 (4-amino-otec-dimethyl-2-ethoxymethyl-1H-imidazo[4,5-c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and the like, etc. Lipopeptides, such as Pam3Cys, are likewise particularly suitable for combining with the pharmaceutical composition described herein (see Deres et al., Nature 1989, 342: 561-564). It is likewise possible for the above-described pharmaceutical composition to contain as (additional) adjuvant a nucleic-acid-based adjuvant, for example CpG and RNA oligonucleotides, etc., or Toll-like receptor ligands, for example ligands of TLR1, TLR2, TLR3, TLR4, TLR5, TLR6, TLR7, TLR8, TLR9, TLR10, TLR11, TLR12 or TLR13 or homologues thereof.

The pharmaceutical composition (of whatever therapeutic use) according to the invention described herein can optionally contain a pharmaceutically acceptable carrier. The expression “pharmaceutically acceptable carrier” used here preferably includes one or more compatible solid or liquid fillers or diluents or encapsulating compounds, which are suitable for administration to a person. The term “compatible” as used here means that the constituents of the composition are capable of being mixed with the base-modified RNA used according to the invention, with the adjuvant that is optionally additionally present, and with one another in such a manner that no interaction occurs which would substantially reduce the pharmaceutical effectiveness of the composition under usual use conditions, such as, for example, reduce the pharmaceutical activity of the encoded pharmaceutically active protein or even inhibit or impair the expression of the pharmaceutically active protein. Pharmaceutically acceptable carriers must, of course, have sufficiently high purity and sufficiently low toxicity to make them suitable for administration to a person to be treated. Some examples of compounds which can be used as pharmaceutically acceptable carriers or constituents thereof are sugars, such as, for example, lactose, glucose and sucrose; starches, such as, for example, corn starch or potato starch; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid; emulsifiers, such as, for example, Tween®; wetting agents, such as, for example, sodium lauryl sulfate; colouring agents; taste-imparting agents, pharmaceutical carriers; tablet-forming agents; stabilisers; antioxidants; preservatives; pyrogen-free water; isotonic saline and phosphate-buffered solutions.

The choice of a pharmaceutically acceptable carrier is determined in principle by the manner in which the pharmaceutical composition used according to the invention is administered. The pharmaceutical composition used according to the invention can be administered, for example, systemically. Routes for administration include, for example, transdermal, oral, parenteral, including subcutaneous or intravenous injections, topical and/or intranasal routes. The suitable amount of the pharmaceutical composition to be used can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Pharmaceutically acceptable carriers for topical application which can be used here include those which are suitable for use in lotions, creams, gels and the like. If the compound is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

According to a particular embodiment, the pharmaceutical composition used here can also be in the form of a vaccine. Without being tied to a theory, vaccination is based on the introduction of an antigen, in the present case the base-modified RNA used according to the invention and coding for (a therapeutically active) protein(s), into the organism, in particular into the cell. The base-modified RNA contained in the pharmaceutical composition used here is translated into the protein that is coded for, i.e. the protein coded for by the base-modified RNA used according to the invention is expressed, resulting in the stimulation of an immune response directed against that protein. In the present case of use as a genetic vaccine for the treatment of cancer or tumour diseases or infectious diseases, the adaptive immune response is achieved, for example, by introduction of the genetic information for a tumour or a pathogenic antigen. As a result, the cancer antigen(s) is/are expressed in the organism, resulting in the triggering of an immune response that is effectively directed against the cancer or tumour cells. Vaccines in connection with the present invention typically comprise a composition as described above for a pharmaceutical composition, the composition of such vaccines being determined in particular by the manner in which they are administered. Vaccines are preferably administered systemically, as described here. Routes for administration of such vaccines typically include transdermal, oral, parenteral, including subcutaneous or intravenous injections, topical and/or intranasal routes. Vaccines as described herein are therefore preferably formulated in liquid or solid form. Further auxiliary substances that can further increase the immunogenicity of the vaccine can optionally also be incorporated into vaccines as described herein above. Advantageously, one or more further such auxiliary substances, as defined hereinbefore, are to be chosen for the vaccines described herein, depending on other properties of the base-modified RNA used according to the invention.

According to a further preferred object of the present invention, the base-modified RNA described herein or a pharmaceutical composition as described herein, particularly preferably the vaccine described herein, is used for the treatment of indications mentioned by way of example hereinbelow. Without implying any limitation, it is possible with the described pharmaceutical composition, particularly preferably with the described vaccine, to treat, for example, diseases or conditions such as, for example, cancer or tumour diseases selected from melanomas, malignant melanomas, colon carcinomas, lymphomas, sarcomas, blastomas, renal carcinomas, gastrointestinal tumours, gliomas, prostate tumours, bladder cancer, rectal tumours, stomach cancer, oesophageal cancer, pancreatic cancer, liver cancer, mammary carcinomas (=breast cancer), uterine cancer, cervical cancer, acute myeloid leukaemia (AML), acute lymphoid leukaemia (ALL), chronic myeloid leukaemia (CML), chronic lymphocytic leukaemia (CLL), hepatomas, various virus-induced tumours such as, for example, papilloma virus-induced carcinomas (e.g. cervical carcinoma=cervical cancer), adenocarcinomas, herpes virus-induced tumours (e.g. Burkitt's lymphoma, EBV-induced B-cell lymphoma), heptatitis B-induced tumours (hepatocell carcinomas), HTLV-1- and HTLV-2-induced lymphomas, acoustic neuroma, lung carcinomas (=lung cancer=bronchial carcinoma), small-cell lung carcinomas, pharyngeal cancer, anal carcinoma, glioblastoma, rectal carcinoma, astrocytoma, brain tumours, retinoblastoma, basalioma, brain metastases, medulloblastomas, vaginal cancer, pancreatic cancer, testicular cancer, Hodgkin's syndrome, meningiomas, Schneeberger disease, hypophysis tumour, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumours, oligodendroglioma, vulval cancer, intestinal cancer, colon carcinoma, oesophageal carcinoma (=Oesophageal cancer), wart involvement, tumours of the small intestine, craniopharyngeomas, ovarian carcinoma, genital tumours, ovarian cancer (=Ovarian carcinoma), pancreatic carcinoma (=pancreatic cancer), endometrial carcinoma, liver metastases, penile cancer, tongue cancer, gall bladder cancer, leukaemia, plasmocytoma, lid tumour, prostate cancer (=prostate tumours), etc., or (viral, bacterial or protozoological) infectious diseases selected from influenza, malaria, SARS, yellow fever, AIDS, Lyme borreliosis, Leishmaniasis, anthrax, meningitis, viral infectious diseases such as AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio (childhood lameness), pseudo-croup, fifth disease, rabies, warts, West Nile fever, chickenpox, cytomegalic virus (CMV), bacterial infectious diseases such as miscarriage (prostate inflammation), anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis (colpitis), soft chancre, and infectious diseases caused by parasites, protozoa or fungi, such as amoebiasis, bilharziosis, Chagas disease, Echinococcus, fish tapeworm, fish poisoning (Ciguatera), fox tapeworm, athlete's foot, canine tapeworm, candidosis, yeast fungus spots, scabies, cutaneous Leishmaniosis, lambliasis (giardiasis), lice, malaria, microscopy, onchocercosis (river blindness), fungal diseases, bovine tapeworm, schistosomiasis, sleeping sickness, porcine tapeworm, toxoplasmosis, trichomoniasis, trypanosomiasis (sleeping sickness), visceral Leishmaniosis, nappy/diaper dermatitis or miniature tapeworm.

Another group of diseases to be treated with the base-modified RNA compositions containing the base-modified RNA of the invention relates to heart and circulatory diseases selected from coronary heart disease, arteriosclerosis, apoplexia, hypertonia, and neuronal diseases selected from Alzheimer's disease, amyotrophic lateral sclerosis, dystonia, epilepsy, multiple sclerosis and Parkinson's disease, and autoimmune diseases selected from type I autoimmune diseases or type II autoimmune diseases or type III autoimmune diseases or type IV autoimmune diseases, such as, for example, multiple sclerosis (MS), rheumatoid arthritis, diabetes, type I diabetes (Diabetes mellitus), systemic lupus erythematosus (SLE), chronic polyarthritis, Basedow's disease, autoimmune forms of chronic hepatitis, colitis ulcerosa, type I allergy diseases, type II allergy diseases, type III allergy diseases, type IV allergy diseases, fibromyalgia, hair loss, Bechterew's disease, Crohn's disease, Myasthenia gravis, neurodermitis, Polymyalgia rheumatica, progressive systemic sclerosis (PSS), psoriasis, Reiter's syndrome, rheumatic arthritis, psoriasis, vasculitis, etc, or type II diabetes.

The base-modified RNA or compositions containing the base-modified RNA may also be used to treat genetic disease, which are caused by genetic defects, e.g. due to gene mutations resulting in loss of protein activity or regulatory mutations which do not allow transcribe or translate the protein. Frequently, these disease lead to metabolic disorders or other symptoms, e.g. muscle dystrophy. Accordingly, the present invention allows to treat these diseases by providing the dysfunctional protein via the base-modified RNA, which allows sufficient level of the protein to be translated due to the increased expression rate. Insofar, the following diseases may be treated: 3-beta-hydroxysteroid dehydrogenase deficiency (type II); 3-ketothiolase deficiency; 6-mercaptopurine sensitivity; Aarskog-Scott syndrome; Abetalipoproteinemia; Acatalasemia; Achondrogenesis; Achondrogenesis-hypochondrogenesis; Achondroplasia; Achromatopsia; Acromesomelic dysplasia (Hunter-Thompson type); ACTH deficiency; Acyl-CoA dehydrogenase deficiency (short-chain, medium chain, long chain); Adenomatous polyposis coli; Adenosin-deaminase deficiency; Adenylosuccinase deficiency; Adhalinopathy; Adrenal hyperplasia, congenital (due to 11-beta-hydroxylase deficiency; due to 17-alpha-hydroxylase deficiency; due to 21-hydroxylase deficiency); Adrenal hypoplasia, congenital, with hypogonadotropic hypogonadism; Adrenogenital syndrome; Adrenoleukodystrophy; Adrenomyeloneuropathy; Afibrinogenemia; Agammaglobulinemia; Alagille syndrome; Albinism (brown, ocular, oculocutaneous, rufous); Alcohol intolerance, acute; Aldolase A deficiency; Aldosteronism, glucocorticoid-remediable; Alexander disease; Alkaptonuria; Alopecia universalis; Alpha-1-antichymotrypsin deficiency; Alpha-methylacyl-CoA racemase deficiency; Alpha-thalassemia/mental retardation syndrome; Alport syndrome; Alzheimer disease-1 (APP-related); Alzheimer disease-3; Alzheimer disease-4; Amelogenesis imperfecta; Amyloid neuropathy (familial, several allelic types); Amyloidosis (Dutch type; Finnish type; hereditary renal; renal; senile systemic); Amytrophic lateral sclerosis; Analbuminemia; Androgen insensitivity; Anemia (Diamond-Blackfan); Anemia (hemolytic, due to PK deficiency); Anemia (hemolytic, Rh-null, suppressor type); Anemia (neonatal hemolytic, fatal and nearfatal); Anemia (sideroblastic, with ataxia); Anemia (sideroblastic/hypochromic); Anemia due to G6PD deficiency; Aneurysm (familial arterial); Angelman syndrome; Angioedema; Aniridia; Anterior segment anomalies and cataract; Anterior segment mesenchymal dysgenesis; Anterior segment mesenchymal dysgenesis and cataract; Antithrombin III deficiency; Anxiety-related personality traits; Apert syndrome; Apnea (postanesthetic); ApoA-I and apoC-III deficiency (combined); Apolipoprotein A-II deficiency; Apolipoprotein B-100 (ligand-defective); Apparent mineralocorticoid excess (hypertension due to); Argininemia; Argininosuccinicaciduria; Arthropathy (progressive pseudorheumatoid, of childhood); Aspartylglucosaminuria; Ataxia (episodic); Ataxia with isolated vitamin E deficiency; Ataxia-telangiectasia; Atelosteogenesis II; ATP-dependent DNA ligase I deficiency; Atrial septal defect with atrioventricular conduction defects; Atrichia with papular lesions; Autism (succinylpurinemic); Autoimmune polyglandular disease, type I; Autonomic nervous system dysfunction; Axenfeld anomaly; Azoospermia; Bamforth-Lazarus syndrome; Bannayan-Zonana syndrome; Barthsyndrome; Bartter syndrome (type 2 or type 3); Basal cell carcinoma; Basal cell nevus syndrome; BCG infection; Beare-Stevenson cutis gyrata syndrome; Becker muscular dystrophy; Beckwith-Wiedemann syndrome; Bernard-Soulier syndrome (type B; type C); Bethlem myopathy; Bile acid malabsorption, primary; Biotinidase deficiency; Bladder cancer; Bleeding disorder due to defective thromboxane A2 receptor; Bloom syndrome; Brachydactyly (type B1 or type C); Branchiootic syndrome; Branchiootorenal syndrome; Breast cancer (invasive intraductal; lobular; male, with Reifenstein syndrome; sporadic); Breast cancer-1 (early onset); Breast cancer-2 (early onset); Brody myopathy; Brugada syndrome; Brunner syndrome; Burkitt lymphoma; Butterfly dystrophy (retinal); C1q deficiency (type A; type B; type C); C1r/C1s deficiency; C1s deficiency, isolated; C2 deficiency; C3 deficiency; C3b inactivator deficiency; C4 deficiency; C8 deficiency, type II; C9 deficiency; Campomelic dysplasia with autosomal sex reversal; Camptodactyly-arthropathy-coxa varapericarditis syndrome; Canavan disease; Carbamoylphosphate synthetase I deficiency; Carbohydrate-deficient glycoprotein syndrome (type I; type Ib; type II); Carcinoid tumor of lung; Cardioencephalomyopathy (fatal infantile, due to cytochrome c oxidase deficiency); Cardiomyopathy (dilated; X-linked dilated; familial hypertrophic; hypertrophic); Carnitine deficiency (systemic primary); Carnitine-acylcarnitine translocase deficiency; Carpal tunnel syndrome (familial); Cataract (cerulean; congenital; crystalline aculeiform; juvenile-onset; polymorphic and lamellar; punctate; zonular pulverulent); Cataract, Coppock-like; CD59 deficiency; Central core disease; Cerebellar ataxia; Cerebral amyloid angiopathy; Cerebral arteriopathy with subcortical infarcts and leukoencephalopathy; Cerebral cavernous malformations-1; Cerebrooculofacioskeletal syndrome; Cerebrotendinous xanthomatosis; Cerebrovascular disease; Ceroid lipofuscinosis (neuronal, variant juvenile type, with granular osmiophilic deposits); Ceroid lipofuscinosis (neuronal-1, infantile); Ceroid-lipofuscinosis (neuronal-3, juvenile); Char syndrome; Charcot-Marie-Tooth disease; Charcot-Marie-Tooth neuropathy; Charlevoix-Saguenay type; Chediak-Higashi syndrome; Chloride diarrhea (Finnish type); Cholestasis (benign recurrent intrahepatic); Cholestasis (familial intrahepatic); Cholestasis (progressive familial intrahepatic); Cholesteryl ester storage disease; Chondrodysplasia punctata (brachytelephalangic; rhizomelic; X-linked dominant; X-linked recessive; Grebe type); Chondrosarcoma; Choroideremia; Chronic granulomatous disease (autosomal, due to deficiency of CYBA); Chronic granulomatous disease (X-linked); Chronic granulomatous disease due to deficiency of NCF-1; Chronic granulomatous disease due to deficiency of NCF-2; Chylomicronemia syndrome, familial; Citrullinemia; classical Cockayne syndrome-1; Cleft lip, cleft jaw, cleft palate; Cleft lip/palate ectodermal dysplasia syndrome; Cleidocranial dysplasia; CMO II deficiency; Coats disease; Cockayne syndrome-2, type B; Coffin-Lowry syndrome; Colchicine resistance; Colon adenocarcinoma; Colon cancer; Colorblindness (deutan; protan; tritan); Colorectal cancer; Combined factor V and VIII deficiency, Combined hyperlipemia (familial); Combined immunodeficiency (X-linked, moderate); Complex I deficiency; Complex neurologic disorder; Cone dystrophy-3; Cone-rod dystrophy 3; Cone-rod dystrophy 6; Cone-rod retinal dystrophy-2; Congenital bilateral absence of vas deferens; Conjunctivitis, ligneous; Contractural arachnodactyly; Coproporphyria; Cornea plana congenita; Corneal clouding; Corneal dystrophy (Avellino type; gelatinous drop-like; Groenouw type I; lattice type I; Reis-Bucklers type); Cortisol resistance; Coumarin resistance; Cowden disease; CPT deficiency, hepatic (type I; type II); Cramps (familial, potassium-aggravated); Craniofacial-deafness-hand syndrome; Craniosynostosis (type 2); Cretinism; Creutzfeldt-Jakob disease; Crigler-Najjar syndrome; Crouzon syndrome; Currarino syndrome; Cutis laxa; Cyclic hematopoiesis; Cyclic ichthyosis; Cylindromatosis; Cystic fibrosis; Cystinosis (nephropathic); Cystinuria (type II; type III); Daltonism; Darier disease; D-bifunctional protein deficiency; Deafness, autosomal dominant 1; Deafness, autosomal dominant 11; Deafness, autosomal dominant 12; Deafness, autosomal dominant 15; Deafness, autosomal dominant 2; Deafness, autosomal dominant 3; Deafness, autosomal dominant 5; Deafness, autosomal dominant 8; Deafness, autosomal dominant 9; Deafness, autosomal recessive 1; Deafness, autosomal recessive 2; Deafness, autosomal recessive 21; Deafness, autosomal recessive 3; Deafness, autosomal recessive 4; Deafness, autosomal recessive 9; Deafness, nonsyndromic sensorineural 13; Deafness, X-linked 1; Deafness, X-linked 3; Debrisoquine sensitivity, Dejerine-Sottas disease; Dementia (familial Danish); Dementia (frontotemporal, with parkinsonism); Dent disease; Dental anomalies; Dentatorubro-pallidoluysian atrophy; Denys-Drash syndrome; Dermatofibrosarcoma protuberans; Desmoid disease; Diabetes insipidus (nephrogenic); Diabetes insipidus (neurohypophyseal); Diabetes mellitus (insulin-resistant); Diabetes mellitus (rare form); Diabetes mellitus (type II); Diastrophic dysplasia; Dihydropyrimidinuria; Dosage-sensitive sex reversal; Doyne honeycomb degeneration of retina; Dubin-Johnson syndrome; Duchenne muscular dystrophy; Dyserythropoietic anemia with thrombocytopenia; Dysfibrinogenemia (alpha type; beta type; gamma type); Dyskeratosis congenita-1; Dysprothrombinemia; Dystonia (DOPAresponsive); Dystonia (myoclonic); Dystonia-1 (torsion); Ectodermal dysplasia; Ectopia lentis; Ectopia pupillae; Ectrodactyly (ectodermal dysplasia, and cleft lip/palate syndrome 3); Ehlers-Danlos syndrome (progeroid form); Ehlers-Danlos syndrome (type I; type II; type III; type IV; type VI; type VII); Elastin Supravalvar aortic stenosis; Elliptocytosis-1; Elliptocytosis-2; Elliptocytosis-3; Ellis-van Creveld syndrome; Emery-Dreifuss muscular dystrophy; Emphysema; Encephalopathy, Endocardial fibroelastosis-2; Endometrial carcinoma; Endplate acetylcholinesterase deficiency; Enhanced S-cone syndrome; Enlarged vestibular aqueduct; Epidermolysis bullosa; Epidermolysis bullosa dystrophica (dominant or recessive); Epidermolysis bullosa simplex; Epidermolytic hyperkeratosis; Epidermolytic palmoplantar keratoderma; Epilepsy (generalize; juvenile; myoclonic; nocturnal frontal lobe; progressive myoclonic); Epilepsy, benign, neonatal (type 1 or type 2); Epiphyseal dysplasia (multiple); Episodic ataxia (type 2); Episodic ataxia/myokymia syndrome; Erythremias (alpha-; dysplasia); Erythrocytosis; Erythrokeratoderma; Estrogen resistance; Exertional myoglobinuria due to deficiency of LDH-A; Exostoses, multiple (type 1; type 2); Exudative vitreoretinopathy, X-linked; Fabry disease; Factor H deficiency; Factor VII deficiency; Factor X deficiency; Factor XI deficiency; Factor XII deficiency; Factor XIIIA deficiency; Factor XIIIB deficiency; Familial Mediterranean fever; Fanconi anemia; Fanconi-Bickel syndrome; Farber lipogranulomatosis; Fatty liver (acute); Favism; Fish-eye disease; Foveal hypoplasia; Fragile X syndrome; Frasier syndrome; Friedreich ataxia; fructose-bisphosphatase Fructose intolerance; Fucosidosis; Fumarase deficiency; Fundus albipunctatus; Fundus flavimaculatus; G6PD deficiency; GABA-transaminase deficiency, Galactokinase deficiency with cataracts; Galactose epimerase deficiency; Galactosemia; Galactosialidosis; GAMT deficiency; Gardner syndrome; Gastric cancer; Gaucher disease; Generalized epilepsy with febrile seizures plus; Germ cell tumors; Gerstmann-Straussler disease; Giant cell hepatitis (neonatal); Giant platelet disorder; Giant-cell fibroblastoma; Gitelman syndrome; Glanzmann thrombasthenia (type A; type B); Glaucoma 1A; Glaucoma 3A; Glioblastoma multiforme; Glomerulosclerosis (focal segmental); Glucose transport defect (blood-brain barrier); Glucose/galactose malabsorption; Glucosidase I deficiency, Glutaricaciduria (type I; type IIB; type IIC); Gluthation synthetase deficiency; Glycerol kinase deficiency; Glycine receptor (alpha-1 polypeptide); Glycogen storage disease I; Glycogen storage disease II; Glycogen storage disease III; Glycogen storage disease IV; Glycogen storage disease VI; Glycogen storage disease VII; Glycogenosis (hepatic, autosomal); Glycogenosis (X-linked hepatic); GM1-gangliosidosis; GM2-gangliosidosis; Goiter (adolescent multinodular); Goiter (congenital); Goiter (nonendemic, simple); Gonadal dysgenesis (XY type); Granulomatosis, septic; Graves disease; Greig cephalopolysyndactyly syndrome; Griscelli syndrome; Growth hormone deficient dwarfism; Growth retardation with deafness and mental retardation; Gynecomastia (familial, due to increased aromatase activity); Gyrate atrophy of choroid and retina with ornithinemia (B6 responsive or unresponsive); Hailey-Hailey disease; Haim-Munk syndrome; Hand-foot-uterus syndrome; Harderoporphyrinuria; HDL deficiency (familial); Heart block (nonprogressive or progressive); Heinz body anemia; HELLP syndrome; Hematuria (familial benign); Heme oxygenase-1 deficiency; Hemiplegic migraine; Hemochromotosis; Hemoglobin H disease; Hemolytic anemia due to ADA excess; Hemolytic anemia due to adenylate kinase deficiency; Hemolytic anemia due to band 3 defect; Hemolytic anemia due to glucosephosphate isomerase deficiency; Hemolytic anemia due to glutathione synthetase deficiency; Hemolytic anemia due to hexokinase deficiency; Hemolytic anemia due to PGK deficiency; Hemolytic-uremic syndrome; Hemophagocytic lymphohistiocytosis; Hemophilia A; Hemophilia B; Hemorrhagic diathesis due to factor V deficiency; Hemosiderosis (systemic, due to aceruloplasminemia); Hepatic lipase deficiency; Hepatoblastoma; Hepatocellular carcinoma; Hereditary hemorrhagic telangiectasia-1; Hereditary hemorrhagic telangiectasia-2; Hermansky-Pudlak syndrome; Heterotaxy (X-linked visceral); Heterotopia (periventricular); Hippel-Lindau syndrome; Hirschsprung disease; Histidine-rich glycoprotein Thrombophilia due to HRG deficiency, HMG-CoA lyase deficiency; Holoprosencephaly-2; Holoprosencephaly-3; Holoprosencephaly-4; Holoprosencephaly-5; Holt-Oram syndrome; Homocystinuria; Hoyeraal-Hreidarsson; HPFH (deletion type or nondeletion type); HPRT-related gout; Huntington disease; Hydrocephalus due to aqueductal stenosis; Hydrops fetalis; Hyperbetalipoproteinemia; Hypercholesterolemia, familial; Hyperferritinemia-cataract syndrome; Hyperglycerolemia; Hyperglycinemia; Hyperimmunoglobulinemia D and periodic fever syndrome; Hyperinsulinism; Hyperinsulinism-hyperammonemia syndrome; Hyperkalemic periodic paralysis; Hyperlipoproteinemia; Hyperlysinemia; Hypermethioninemia (persistent, autosomal, dominant, due to methionine, adenosyltransferase I/III deficiency); Hyperornithinemia-hyperammonemiahomocitrullinemia syndrome; Hyperoxaluria; Hyperparathyroidism; Hyperphenylalaninemia due to pterin-4acarbinolamine dehydratase deficiency; Hyperproinsulinemia; Hyperprolinemia; Hypertension; Hyperthroidism (congenital); Hypertriglyceridemia; Hypoalphalipoproteinemia; Hypobetalipoproteinemia; Hypocalcemia; Hypochondroplasia; Hypochromic microcytic anemia; Hypodontia; Hypofibrinogenemia; Hypoglobulinemia and absent B cells; Hypogonadism (hypergonadotropic); Hypogonadotropic (hypogonadism); Hypokalemic periodic paralysis; Hypomagnesemia; Hypomyelination (congenital); Hypoparathyroidism; Hypophosphatasia (adult; childhood; infantile; hereditary); Hypoprothrombinemia; Hypothyroidism (congenital; hereditary congenital; nongoitrous); Ichthyosiform erythroderma; Ichthyosis; Ichthyosis bullosa of Siemens; IgG2 deficiency; Immotile cilia syndrome-1; Immunodeficiency (T-cell receptor/CD3 complex); Immunodeficiency (X-linked, with hyper-IgM); Immunodeficiency due to defect in CD3-gamma; immunodeficiency-centromeric instabilityfacial anomalies syndrome; Incontinentia pigmenti; Insensitivity to pain (congenital, with anhidrosis); Insomnia (fatal familial); Interleukin-2 receptor deficiency (alpha chain); Intervertebral disc disease; Iridogoniodysgenesis; Isolated growth hormone deficiency (Illig type with absent GH and Kowarski type with bioinactive GH); Isovalericacidemia; Jackson-Weiss syndrome; Jensen syndrome; Jervell and Lange-Nielsen syndrome; Joubert syndrome; Juberg-Marsidi syndrome; Kallmann syndrome; Kanzaki disease; Keratitis; Keratoderma (palmoplantar); Keratosis palmoplantaris striata I; Keratosis palmoplantaris striata II; Ketoacidosis due to SCOT deficiency, Keutel syndrome; Klippel-Trenaurnay syndrome; Kniest dysplasia; Kostmann neutropenia; Krabbe disease; Kurzripp-Polydaktylie syndrome; Lacticacidemia due to PDX1 deficiency; Langer mesomelic dysplasia; Laron dwarfism; Laurence-Moon-Biedl-Bardet syndrome; LCHAD deficiency, Leber congenital amaurosis; Left-right axis malformation; Leigh syndrome; Leiomyomatosis (diffuse, with Alport syndrome); Leprechaunism; Leri-Weill dyschondrosteosis; Lesch-Nyhan syndrome; Leukemia (acute myeloid; acute promyelocytic; acute T-cell lymphoblastic; chronic myeloid; juvenile myelomonocytic; Leukemia-1 (T-cell acute lymphocytic); Leukocyte adhesion deficiency; Leydig cell adenoma; Lhermitte-Duclos syndrome; Liddle syndrome; L1-Fraumeni syndrome; Lipoamide dehydrogenase deficiency; Lipodystrophy; Lipoid adrenal hyperplasia; Lipoprotein lipase deficiency; Lissencephaly (X-linked); Lissencephaly-1; liver Glycogen storage disease (type 0); Long QT syndrome-1; Long QT syndrome-2; Long QT syndrome-3; Long QT syndrome-5; Long QT syndrome-6; Lowe syndrome; Lung cancer; Lung cancer (nonsmall cell); Lung cancer (small cell); Lymphedema; Lymphoma (B-cell non-Hodgkin); Lymphoma (diffuse large cell); Lymphoma (follicular); Lymphoma (MALT); Lymphoma (mantel cell); Lymphoproliferative syndrome (X-linked); Lysinuric protein intolerance; Machado-Joseph disease; Macrocytic anemia refractory (of 5q syndrome); Macular dystrophy; Malignant mesothelioma; Malonyl-CoA decarboxylase deficiency; Mannosidosis, (alpha- or beta-); Maple syrup urine disease (type Ia; type Ib; type II); Marfan syndrome; Maroteaux-Lamy syndrome; Marshall syndrome; MASA syndrome; Mast cell leukemia; Mastocytosis with associated hematologic disorder; McArdle disease; McCune-Albright polyostotic fibrous dysplasia; McKusick-Kaufman syndrome; McLeod phenotype; Medullary thyroid carcinoma; Medulloblastoma; Meesmann corneal dystrophy; Megaloblastic anemia-1; Melanoma; Membroproliferative glomerulonephritis; Meniere disease; Meningioma (NF2-related; SIS-related); Menkes disease; Mental retardation (X-linked); Mephenyloin poor metabolizer; Mesothelioma; Metachromatic leukodystrophy; Metaphyseal chondrodysplasia (Murk Jansen type; Schmid type); Methemoglobinemia; Methionine adenosyltransferase deficiency (autosomal recessive); Methylcobalamin deficiency (cbl G type); Methylmalonicaciduria (mutase deficiency type); Mevalonicaciduria; MHC class II deficiency; Microphthalmia (cataracts, and iris abnormalities); Miyoshi myopathy; MODY; Mohr-Tranebjaerg syndrome; Molybdenum cofactor deficiency (type A or type B); Monilethrix; Morbus Fabry; Morbus Gaucher; Mucopolysaccharidosis; Mucoviscidosis; Muencke syndrome; Muir-Torre syndrome; Mulibrey nanism; Multiple carboxylase deficiency (biotinresponsive); Multiple endocrine neoplasia; Muscle glycogenosis; Muscular dystrophy (congenital merosindeficient); Muscular dystrophy (Fukuyama congenital); Muscular dystrophy (limb-girdle); Muscular dystrophy) Duchenne-like); Muscular dystrophy with epidermolysis bullosa simplex; Myasthenic syndrome (slow-channel congenital); Mycobacterial infection (atypical, familial disseminated); Myelodysplastic syndrome; Myelogenous leukemia; Myeloid malignancy; Myeloperoxidase deficiency; Myoadenylate deaminase deficiency; Myoglobinuria/hemolysis due to PGK deficiency; Myoneurogastrointestinal encephalomyopathy syndrome; Myopathy (actin; congenital; desmin-related; cardioskeletal; distal; nemaline); Myopathy due to CPT II deficiency; Myopathy due to phosphoglycerate mutase deficiency; Myotonia congenita; Myotonia levior; Myotonic dystrophy; Myxoid liposarcoma; NAGA deficiency; Nailpatella syndrome; Nemaline myopathy 1 (autosomal dominant); Nemaline myopathy 2 (autosomal recessive); Neonatal hyperparathyroidism; Nephrolithiasis; Nephronophthisis (juvenile); Nephropathy (chronic hypocomplementemic); Nephrosis-1; Nephrotic syndrome; Netherton syndrome; Neuroblastoma; Neurofibromatosis (type 1 or type 2); Neurolemmomatosis; neuronal-5 Ceroid-lipofuscinosis; Neuropathy; Neutropenia (alloimmune neonatal); Niemann-Pick disease (type A; type B; type C1; type D); Night blindness (congenital stationary); Nijmegen breakage syndrome; Noncompaction of left ventricular myocardium; Nonepidermolytic palmoplantar keratoderma; Norrie disease; Norum disease; Nucleoside phosphorylase deficiency; Obesity; Occipital hornsyndrome; Ocular albinism (Nettleship-Falls type); Oculopharyngeal muscular dystorphy; Oguchi disease; Oligodontia; Omenn syndrome; Opitz G syndrome; Optic nerve coloboma with renal disease; Ornithine transcarbamylase deficiency; Oroticaciduria; Orthostatic intolerance; OSMED syndrome; Ossification of posterior longitudinal ligament of spine; Osteoarthrosis; Osteogenesis imperfecta; Osteolysis; Osteopetrosis (recessive or idiopathic); Osteosarcoma; Ovarian carcinoma; Ovarian dysgenesis; Pachyonychia congenita (Jackson-Lawler type or Jadassohn-Lewandowsky type); Paget disease of bone; Pallister-Hall syndrome; Pancreatic agenesis; Pancreatic cancer; Pancreatitis; Papillon-Lefevre syndrome; Paragangliomas; Paramyotonia congenita; Parietal foramina; Parkinson disease (familial or juvenile); Paroxysmal nocturnal hemoglobinuria; Pelizaeus-Merzbacher disease; Pendred syndrome; Perineal hypospadias; Periodic fever; Peroxisomal biogenesis disorder; Persistent hyperinsulinemic hypoglycemia of infancy; Persistent Mullerian duct syndrome (type II); Peters anomaly; Peutz-Jeghers syndrome; Pfeiffer syndrome; Phenylketonuria; Phosphoribosyl pyrophosphate synthetaserelated gout; Phosphorylase kinase deficiency of liver and muscle; Piebaldism; Pilomatricoma; Pinealoma with bilateral retinoblastoma; Pituitary ACTH secreting adenoma; Pituitary hormone deficiency; Pituitary tumor; Placental steroid sulfatase deficiency; Plasmin inhibitor deficiency; Plasminogen deficiency (types I and II); Plasminogen Tochigi disease; Platelet disorder; Platelet glycoprotein IV deficiency; Platelet-activating factor acetylhydrolase deficiency; Polycystic kidney disease; Polycystic lipomembranous osteodysplasia with sclerosing leukenencephalophathy; Polydactyl), postaxial; Polyposis; Popliteal pterygium syndrome; Porphyria (acute hepatic or acute intermittent or congenital erythropoietic); Porphyria cutanea tarda; Porphyria hepatoerythropoietic; Porphyria variegata; Prader-Willi syndrome; Precocious puberty; Premature ovarian failure; Progeria Typ I; Progeria Typ II; Progressive external opthalmoplegia; Progressive intrahepatic cholestasis-2; Prolactinoma (hyperparathyroidism, carcinoid syndrome); Prolidase deficiency; Propionicacidemia; Prostate cancer; Protein S deficiency; Proteinuria; Protoporphyria (erythropoietic); Pseudoachondroplasia; Pseudohermaphroditism; Pseudohypoaldosteronism; Pseudohypoparathyroidism; Pseudovaginal perineoscrotal hypospadias; Pseudovitamin D deficiency rickets; Pseudoxanthoma elasticum (autosomal dominant; autosomal recessive); Pulmonary alveolar proteinosis; Pulmonary hypertension; Purpura fulminans; Pycnodysostosis; Pyropoikilocytosis; Pyruvate carboxylase deficiency; Pyruvate dehydrogenase deficiency; Rabson-Mendenhall syndrome; Refsum disease; Renal cell carcinoma; Renal tubular acidosis; Renal tubular acidosis with deafness; Renal tubular acidosis-osteopetrosis syndrome; Reticulosis (familial histiocytic); Retinal degeneration; Retinal dystrophy; Retinitis pigmentosa; Retinitis punctata albescens; Retinoblastoma; Retinol binding protein deficiency; Retinoschisis; Rett syndrome; Rh(mod) syndrome; Rhabdoid predisposition syndrome; Rhabdoid tumors; Rhabdomyosarcoma; Rhabdomyosarcoma (alveolar); Rhizomelic chondrodysplasia punctata; Ribbing-Syndrome; Rickets (vitamin D-resistant); Rieger anomaly; Robinow syndrome; Rothmund-Thomson syndrome; Rubenstein-Taybi syndrome; Saccharopinuria; Saethre-Chotzen syndrome; Salla disease; Sandhoff disease (infantile, juvenile, and adult forms); Sanfilippo syndrome (type A or type B); Schindler disease; Schizencephaly; Schizophrenia (chronic); Schwannoma (sporadic); SCID (autosomal recessive, T-negative/B positive type); Secretory pathway w/TMD; SED congenita; Segawa syndrome; Selective T-cell defect; SEMD (Pakistani type); SEMD (Strudwick type); Septooptic dysplasia; Severe combined immunodeficiency (B cell negative); Severe combined immunodeficiency (T-cell negative, B-cell/natural killer cell-positive type); Severe combined immunodeficiency (Xlinked); Severe combined immunodeficiency due to ADA deficiency; Sex reversal (XY, with adrenal failure); Sezary syndrome; Shah-Waardenburg syndrome; Short stature; Shprintzen-Goldberg syndrome; Sialic acid storage disorder; Sialidosis (type I or type II); Sialuria; Sickle cell anemia; Simpson-Golabi-Behmel syndrome; Situs ambiguus; Sjogren-Larsson syndrome; Smith-Fineman-Myers syndrome; Smith-Lemli-Opitz syndrome (type I or type II); Somatotrophinoma; Sorsby fundus dystrophy; Spastic paraplegia; Spherocytosis; Spherocytosis-1; Spherocytosis-2; Spinal and bulbar muscular atrophy of Kennedy; Spinal muscular atrophy; Spinocerebellar ataxia; Spondylocostal dysostosis; Spondyloepiphyseal dysplasia tarda; Spondylometaphyseal dysplasia (Japanese type); Stargardt disease-1; Steatocystoma multiplex; Stickler syndrome; Sturge-Weber syndrome; Subcortical laminal heteropia; Subcortical laminar heterotopia; Succinic semialdehyde dehydrogenase deficiency; Sucrose intolerance; Sutherland-Haan syndrome; Sweat chloride elevation without CF; Symphalangism; Synostoses syndrome; Synpolydactyly; Tangier disease; Tay-Sachs disease; T-cell acute lymphoblastic leukemia; T-cell immunodeficiency; T-cell prolymphocytic leukemia; Thalassemia (alpha- or delta-); Thalassemia due to Hb Lepore; Thanatophoric dysplasia (types I or II); Thiamine-responsive megaloblastic anemia syndrome; Thrombocythemia; Thrombophilia (dysplasminogenemic); Thrombophilia due to heparin cofactor II deficiency; Thrombophilia due to protein C deficiency; Thrombophilia due to thrombomodulin defect; Thyroid adenoma; Thyroid hormone resistance; Thyroid iodine peroxidase deficiency; Tietz syndrome; Tolbutamide poor metabolizer; Townes-Brocks syndrome; Transcobalamin II deficiency; Treacher Collins mandibulofacial dysostosis; Trichodontoosseous syndrome; Trichorhinophalangeal syndrome; Trichothiodystrophy; Trifunctional protein deficiency (type I or type II); Trypsinogen deficiency; Tuberous sclerosis-1; Tuberous sclerosis-2; Turcot syndrome; Tyrosine phosphatase; Tyrosinemia; Ulnar-mammary syndrome; Urolithiasis (2,8-dihydroxyadenine); Usher syndrome (type 1B or type 2A); Venous malformations; Ventricular tachycardia; Virilization; Vitamin K-dependent coagulation defect; VLCAD deficiency; Vohwinkel syndrome; von Hippel-Lindau syndrome; von Willebrand disease; Waardenburg syndrome; Waardenburg syndrome/ocular albinism; Waardenburg-Shah neurologic variant; Waardenburg-Shah syndrome; Wagner syndrome; Warfarin sensitivity; Watson syndrome; Weissenbacher-Zweymuller syndrome; Werner syndrome; Weyers acrodental dysostosis; White sponge nevus; Williams-Beuren syndrome; Wilms tumor (type 1); Wilson disease; Wiskott-Aldrich syndrome; Wolcott-Rallison syndrome; Wolfram syndrome; Wolman disease; Xanthinuria (type I); Xeroderma pigmentosum; X-SCID; Yemenite deaf-blind hypopigmentation syndrome; ypocalciuric hypercalcemia (type I); Zellweger syndrome; Zlotogora-Ogur syndrome;

Preferred diseases to be treated which have a genetic inherited background and which are typically caused by a single gene defect and are inherited according to Mendel's laws are preferably selected from the group consisting of autosomal-recessive inherited diseases, such as, for example, adenosine deaminase deficiency, familial hypercholesterolaemia, Canavan's syndrome, Gaucher's disease, Fanconi anaemia, neuronal ceroid lipofuscinoses, mucoviscidosis (cystic fibrosis), sickle cell anaemia, phenylketonuria, alcaptonuria, albinism, hypothyreosis, galactosaemia, alpha-1-anti-trypsin deficiency, Xeroderma pigmentosum, Ribbing's syndrome, mucopolysaccharidoses, cleft lip, jaw, palate, Laurence Moon Biedl Bardet sydrome, short rib polydactylia syndrome, cretinism, Joubert's syndrome, type II progeria, brachydactylia, adrenogenital syndrome, and X-chromosome inherited diseases, such as, for example, colour blindness, e.g. red/green blindness, fragile X syndrome, muscular dystrophy (Duchenne and Becker-Kiener type), haemophilia A and B, G6PD deficiency, Fabry's disease, mucopolysaccharidosis, Norrie's syndrome, Retinitis pigmentosa, septic granulomatosis, X-SCID, ornithine transcarbamylase deficiency, Lesch-Nyhan syndrome, or from autosomal-dominant inherited diseases, such as, for example, hereditary angiooedema, Marfan syndrome, neurofibromatosis, type I progeria, Osteogenesis imperfecta, Klippel-Trenaumay syndrome, Sturge-Weber syndrome, Hippel-Lindau syndrome and tuberosis sclerosis.

The present invention may also provide therapeutic approaches to treat autoimmune diseases. Accordingly, the base-modified RNA or a composition containing a base-modified RNA may be used for the treatment of for the preparation of a medicament for the treatment of autoimmune diseases. Autoimmune diseases can be broadly divided into systemic and organ-specific or localised autoimmune disorders, depending on the principal clinico-pathologic features of each disease. Autoimmune disease may be divided into the categories of systemic syndromes, including systemic lupus erythematosus (SLE), Sjögren's syndrome, Scleroderma, Rheumatoid Arthritis and polymyositis or local syndromes which may be endocrinologic (type I diabetes (Diabetes mellitus Type 1), Hashimoto's thyroiditis, Addison's disease etc.), dermatologic (pemphigus vulgaris), haematologic (autoimmune haemolytic anaemia), neural (multiple sclerosis) or can involve virtually any circumscribed mass of body tissue. The autoimmune diseases to be treated may be selected from the group consisting of type I autoimmune diseases or type II autoimmune diseases or type III autoimmune diseases or type IV autoimmune diseases, such as, for example, multiple sclerosis (MS), rheumatoid arthritis, diabetes, type I diabetes (Diabetes mellitus Type 1), chronic polyarthritis, Basedow's disease, autoimmune forms of chronic hepatitis, colitis ulcerosa, type I allergy diseases, type II allergy diseases, type III allergy diseases, type IV allergy diseases, fibromyalgia, hair loss, Bechterew's disease, Crohn's disease, Myasthenia gravis, neurodermitis, Polymyalgia rheumatica, progressive systemic sclerosis (PSS), Reiter's syndrome, rheumatic arthritis, psoriasis, vasculitis, etc, or type II diabetes. While the exact mode as to why the immune system induces a immune reaction against autoantigens has not been elucidated so far, there are several findings with regard to the etiology. Accordingly, the autoreaction may be due to a T-Cell bypass. A normal immune system requires the activation of B-cells by T-cells before the former can produce antibodies in large quantities. This requirement of a T-cell can be by-passed in rare instances, such as infection by organisms producing super-antigens, which are capable of initiating polyclonal activation of B-cells, or even of T-cells, by directly binding to the β-subunit of T-cell receptors in a non-specific fashion. Another explanation deduces autoimmune diseases from a Molecular Mimicry. An exogenous antigen may share structural similarities with certain host antigens; thus, any antibody produced against this antigen (which mimics the self-antigens) can also, in theory, bind to the host antigens and amplify the immune response. The most striking form of molecular mimicry is observed in Group A beta-haemolytic streptococci, which shares antigens with human myocardium, and is responsible for the cardiac manifestations of Rheumatic Fever. The present invention allows therefore to provide an inventive composition containing containing an base-modified RNA coding for an autoantigen, which typically allows the immune system to be desensitized, or may also provide an (immunostimulatory) composition according to the invention (which does not contain an autoantigen).

The invention therefore relates also to the use of a base-modified RNA as described herein, or of a pharmaceutical composition as described herein, particularly preferably the vaccine described herein, for the treatment of indications or diseases mentioned above. It also includes in particular the use of the base-modified RNA described herein for inoculation or the use of the described pharmaceutical composition as an inoculant.

According to a further object of the present invention, a method for treating the above-mentioned diseases, or an inoculation method for preventing the above-mentioned diseases, is provided, which method comprises administering the described pharmaceutical composition to a patient, in particular to a human being.

The present invention relates also to an in vitro transcription method for the preparation of base-modified RNA, comprising the following steps:
a) preparation/provision of a nucleic acid coding for a protein of interest, in particular as described above;
b) addition of the (desoxy)ribonucleic acid to an in vitro transcription medium comprising a RNA polymerase, a suitable buffer, a nucleic acid mix, comprising one or more base-modified nucleotides as described above as replacement for one or more of the naturally occurring nucleotides A, G, C and/or U, and optionally one or more naturally occurring nucleotides A, G, C or U if not all of the naturally occurring nucleotides A, G, C or U are to be replaced, and optionally a RNase inhibitor;
c) incubation of the nucleic acid in the in vitro transcription medium and in vitro transcription of the nucleic acid;
d) optional purification and removal of the unincorporated nucleotides from the in vitro transcription medium.

A nucleic acid as described in step a) of the in vitro transcription method according to the invention can be any nucleic acid as described above that codes for a protein of interest, in particular as mentioned herein, preferably a diagnostically relevant protein, a therapeutically active protein, or any other protein used or usable for laboratory or research purposes. There are used for this purpose typically DNA sequences, for example genomic DNA or fragments thereof, or plasmids, coding for a protein as described above, or RNA sequences (corresponding thereto), for example mRNA sequences, preferably in linearised form. The in vitro transcription can usually be carried out using a vector having a RNA polymerase binding site. To this end there can be used any vectors known in the art, for example commercially available vectors (see above). Preference is given, for example, to those vectors that have a SP6 or a T7 or T3 binding site upstream and/or downstream of the cloning site. Accordingly, the nucleic acid sequences used can be transcribed later, as desired, depending on the chosen RNA polymerase. A nucleic acid sequence used for in vitro transcription and coding for a protein as defined above is typically cloned into a vector, for example via a multiple cloning site of the vector used. Before the transcription, the clone is typically cleaved with restriction enzymes at the site at which the future 3′ end of the RNA is to be located, using a suitable restriction enzyme, and the fragment is purified. This prevents the RNA from containing vector sequences, and a RNA of defined length is obtained. It is preferred not to use any restriction enzymes that produce 3′-protruding ends (such as, for example, Aat II, Apa I, Ban II, Bgl I, Bsp 1286, BstX I, Cfo I, Hae II, HgiA I, Hha I, Kpn I, Pst I, Pvu I, Sac I, Sac II, Sfi I, Sph I, etc.). If such restriction enzymes are nevertheless to be used, the 3′-protruding end is preferably filled, for example with Klenow or T4-DNA polymerase.

Alternatively, it is also possible to prepare the nucleic acid as transcription template by polymerase chain reaction (PCR). To this end, one of the primers used typically contains the sequence of a RNA polymerase binding site. It is further preferred for the 5′ end of the primer used to have a length of approximately from 10 to 50 further nucleotides, more preferably from 15 to 30 further nucleotides and most preferably of approximately 20 nucleotides.

Prior to the in vitro transcription, the nucleic acid, for example the nucleic acid, e.g. the DNA or RNA template, is typically purified and freed of RNase in order to ensure a high yield. Purification can be carried out by any process known in the art, for example with a caesium chloride gradient or ion-exchange process.

According to method step b), the nucleic acid is added to an in vitro transcription medium. A suitable in vitro transcription medium first contains a nucleic acid as prepared under step a), for example approximately from 0.1 to 10 μg, preferably approximately from 1 to 5 μg, more preferably 2.5 μg and most preferably approximately 1 μg, of such a nucleic acid. A suitable in vitro transcription medium further optionally contains a reducing agent, e.g. DTT, more preferably approximately from 1 to 20 μl of 50 mM DTT, yet more preferably approximately 5 μl of 50 mM DTT. The in vitro transcription medium further contains nucleotides, for example a nucleotide mix, in the case of the present invention consisting of base-modified nucleotides as defined above (typically approximately from 0.1 to 10 mM per nucleotide, preferably from 0.1 to 1 mM per nucleotide, preferably approximately 4 mM in total), and optionally unmodified nucleotides. Base-modified nucleotides as described above (approximately 1 mM per nucleotide, preferably approximately 4 mM in total), e.g. pseudouridine-5′-triphosphate, 5-methylcytidine-5′-triphosphate, etc., are typically added in such an amount that the base-modified nucleotide is replaced completely by the native nucleotide. It is, however, also possible to use mixtures of one or more base-modified nucleotides and one or more naturally occurring nucleotides instead of a particular nucleotide, that is to say one or more base-modified nucleotides as described above can occur as a replacement for one or more of the naturally occurring nucleotides A, G, C or U and optionally additionally one or more naturally occurring nucleotides A, G, C or U, if not all the naturally occurring nucleotides A, G, C or U are to be replaced. By selective addition of the desired base to the in vitro transcription medium, the content, that is to say the occurrence and amount, of the desired base modification in the transcribed base-modified RNA sequence can therefore be controlled. A suitable in vitro transcription medium likewise contains a RNA polymerase, e.g. T7-RNA polymerase (e.g. T7-Opti mRNA Kit, CureVac, Tübingen, Germany), T3-RNA polymerase or SP6, typically approximately from 10 to 500 U, preferably approximately from 25 to 250 U, more preferably approximately from 50 to 150 U, and most preferably approximately 100 U of RNA polymerase. The in vitro transcription medium is further preferably kept free of RNase in order to avoid degradation of the transcribed RNA. A suitable in vitro transcription medium therefore optionally contains in addition a RNase inhibitor.

In a step c), the nucleic acid is incubated and transcribed in the in vitro transcription medium, typically for approximately from 30 to 120 minutes, preferably for approximately from 40 to 90 minutes and most preferably for approximately 60 minutes, at approximately from 30 to 45° C., preferably at from 37 to 42° C. The incubation temperature is governed by the RNA polymerase that is used, for example in the case of T7 RNA polymerase it is approximately 37° C. The nucleic acid obtained by the transcription is preferably a RNA, more preferably a mRNA.

After the incubation, purification of the reaction can optionally take place in step d) of the in vitro transcription method according to the invention. To this end, any suitable process known in the art can be used, for example chromatographic purification processes, e.g. affinity chromatography, gel filtration, etc. By means of the purification, non-incorporated, i.e. excess, nucleotides can be removed from the in vitro transcription medium.

The present invention relates also to an in vitro transcription and translation method for increasing the expression of a protein, comprising the following steps:
a) preparation/provision of a nucleic acid coding for a protein of interest, in particular as described above;
b) addition of the nucleic acid to an in vitro transcription medium comprising a RNA polymerase, a suitable buffer, a nucleic acid mix, comprising one or more base-modified nucleotides as described above as replacement for one or more of the naturally occurring nucleotides A, G, C and/or U, and optionally one or more naturally occurring nucleotides A, G, C or U if not all the naturally occurring nucleotides A, G, C or U are to be replaced, and optionally a RNase inhibitor;
c) incubation of the nucleic acid in the in vitro transcription medium and in vitro transcription of the nucleic acid;
d) optional purification and removal of the unincorporated nucleotides from the in vitro transcription medium;
e) addition of the base-modified nucleic acid obtained in step c) (and optionally in step d)) to an in vitro translation medium;
f) incubation of the base-modified nucleic acid in the in vitro translation medium and in vitro translation of the protein coded for by the base-modified nucleic acid;
g) optional purification of the protein translated in step f).

Steps a), b), c) and d) of the in vitro transcription and translation method according to the invention for increasing the expression of a protein are identical with steps a), b), c) and d) of the above-described in vitro transcription method according to the invention.

In step e) of the in vitro transcription and translation method according to the invention for increasing the expression of a protein, the base-modified nucleic acid obtained in step c) (and optionally in step d)) is added to a suitable in vitro translation medium. A suitable in vitro translation medium comprises, for example, reticulocyte lysate, wheatgerm extract, etc. Such a medium conventionally further comprises an amino acid mix. The amino acid mix typically comprises (all) naturally occurring amino acids and, optionally, modified amino acids, e.g. ³⁵S-methionine (e.g. for controlling the translation efficiency via autoradiography). A suitable in vitro translation medium further comprises a reaction buffer. In vitro translation media are described, for example, by Krieg and Melton (1987) (P. A. Krieg and D. A. Melton (1987) In vitro RNA synthesis with SP6 RNA polymerase Methods Enzymol 155:397-415), the disclosure of which is incorporated into the present invention by reference in its entirety.

In a step f) of the in vitro transcription and translation method according to the invention for increasing the expression of a protein, the base-modified nucleic acid is incubated in the in vitro translation medium, and the protein coded for by the base-modified nucleic acid is translated in vitro. The incubation time is typically approximately from 30 to 120 minutes, preferably approximately from 40 to 90 minutes and most preferably approximately 60 minutes. The incubation temperature is typically in a range of approximately from 20 to 40° C., preferably approximately from 25 to 35° C. and most preferably approximately 30° C.

Steps b) to f) of the in vitro transcription and translation method according to the invention for increasing the expression of a protein, or individual steps of steps b) to f), can be combined with one another, that is to say can be carried out together. It is preferred to add all the necessary components together at the beginning or to add them to the reaction medium in succession during the reaction according to the sequence of the described steps b) to f).

In an optional step g), the translated protein obtained in step f) can be purified. Purification can be carried out by processes known to a person skilled in the art from the art, for example chromatography, such as, for example, affinity chromatography (HPLC, FPLC, etc.), ion-exchange chromatography, gel chromatography, size exclusion chromatography, gas chromatography, or antibody detection, or biophysical processes, such as, for example, NMR analyses, etc. (see e.g. Maniatis et al. (2001) supra). Chromatography processes, including affinity chromatography processes, can suitably use tags for purification, as described above, for example a hexahistidine tag (HIS tag, polyhistidine tag), a streptavidin tag (strep tag), a SBP tag (streptavidin binding tag), a GST (glutathione S-transferase) tag, etc.). The purification can further take place via an antibody epitope, (antibody binding tag), for example a Myc tag, a Swal 1 epitope, a FLAG tag, a HA tag, etc., that is to say by recognition of the epitope via the (immobilised) antibody.

The present invention relates also to an in vitro transcription and translation method for increasing the expression of a protein in a host cell, comprising the following steps:
a) preparation/procision of a (desoxy)ribonucleic acid coding for a protein of interest, in particular as described above;
b) addition of the nucleic acid to an in vitro transcription medium comprising a RNA polymerase, a suitable buffer, one or more base-modified nucleotides as described above as replacement for one or more of the naturally occurring nucleotides A, G, C and/or U, and optionally one or more naturally occurring nucleotides A, G, C or U if not all the naturally occurring nucleotides A, G, C or U are to be replaced;
c) incubation of the nucleic acid in the in vitro transcription medium and in vitro transcription of the nucleic acid;
d) optional purification and removal of the unincorporated nucleotides from the in vitro transcription medium;
e′) transfection of the base-modified nucleic acid obtained in step c) (and optionally d)) into a host cell;
f′) incubation of the base-modified nucleic acid in the host cell and translation of the protein coded for by the base-modified nucleic acid in the host cell;
g′) optional isolation and/or purification of the protein translated in step f′).

Steps a), b), c) and d) of the in vitro transcription and translation method for increasing the expression of a protein in a host cell are identical with steps a), b), c) and d) of the above-described in vitro transcription method according to the invention and of the above-described in vitro transcription and translation method according to the invention for increasing the expression of a protein.

According to step e′) of the in vitro transcription and translation method according to the invention, the transfection of the base-modified nucleic acid obtained in step c) (and optionally d)) into a host cell takes place. The transfection is generally carried out by transfection methods known in the art (see e.g. Maniatis et al. (2001) Molecular Cloning: A laboratory manual, Cold Spring Harbor Laboratory Press, Cold Spring Harbor, N.Y.). Suitable transfection methods include, without implying any limitation, for example electroporation methods, including modified electroporation methods (e.g. nucleofection), calcium phosphate methods, e.g. the calcium co-precipitation method, the DEAE-dextran method, the lipofection method, e.g. the transferrin-mediated lipofection method, polyprene transfection, particle bombardment, nanoplexes, e.g. PLGA, polyplexes, e.g. PEI, protoplast fusion and the microinjection method, the lipofection method in particular having been found to be a suitable method.

In connection with step e′) of the in vitro transcription and translation method according to the invention for increasing the expression of a protein in a host cell, a (suitable) host cell includes any cell that permits expression of the base-modified RNA used according to the invention, preferably any cultivated eukaryotic cell (e.g. yeast cells, plant cells, animal cells and human cells) or prokaryotic cell (bacterial cells). Cells of multicellular organisms are preferably chosen for the expression of the protein coded for by the base-modified RNA used according to the invention, if posttranslational modifications, e.g. glycosylation of the encoded protein, are required (N- and/or O-coupled). Unlike prokaryotic cells, such (higher) eukaryotic cells permit the posttranslational modification of the synthesised protein. The person skilled in the art knows a large number of such higher eukaryotic cells or cell lines. e.g. 293T (embryonic liver cell line), HeLa (human cervical carcinoma cells), CHO (cells from the ovaries of Chinese hamsters) and further cell lines, including cells and cell lines developed for laboratory purposes, such as, for example, hTERT-MSC, HEK293, Sf9 or COS cells. Suitable eukaryotic cells further include cells or cell lines that are impaired by diseases or infections, for example cancer cells, in particular cancer cells of any of the cancer types mentioned herein in the description, cells impaired by HIV and/or cells of the immune system or of the central nervous system (CNS). Particularly preferred eukaryotic cells are human cells or animal cells. Suitable host cells can likewise be derived from eukaryotic microorganisms such as yeast, e.g. Saccharomyces cerevisiae (Stinchcomb et al., Nature, 282:39, (1997)), Schizosaccharomyces pombe, Candida, Pichia, and filamentous fungi of the genera Aspergillus, Penicillium, etc. Suitable host cells likewise include prokaryotic cells, such as, for example, bacterial cells, for example from Escherichia coli or from bacteria of the genera Bacillus, Lactococcus, Lactobacillus, Pseudomonas, Streptomyces, Streptococcus, Staphylococcus, preferably E. coli, etc.

In step f′) of the in vitro transcription and translation method according to the invention for increasing the expression of a protein in a host cell, incubation of the base-modified nucleic acid in the host cell and translation of the protein coded for the by base-modified nucleic acid in the host cell take place. To this end, expression mechanisms inherent in the host cell are preferably used, e.g. by translation of the (m)RNA in the host cell via ribosomes and tRNAs. The incubation temperatures used thereby are governed by the host cell systems used in a particular case.

In an optional step g′), the translated protein obtained in step f′) can be isolated and/or purified. Isolation of the translated (expressed) protein typically comprises separating the protein from reaction constituents and can be carried out by processes known to a person skilled in the art, for example by cell lysis, ultrasonic decomposition, or similar methods. Purification can be carried out by methods as described for step e) of the in vitro transcription and translation method according to the invention for increasing the expression of a protein.

Independently of steps (a) to (d), the nucleic acid used according to the invention can also be expressed by an in vitro translation method of steps (e′) to (g′), which, as such, also forms part of the present invention.

The present invention relates also to an in vitro transcription and in vivo translation method for increasing the expression of a (therapeutically active) protein in an organism, comprising the following steps:
a) preparation/provision of a (desoxy)ribonucleic acid coding for a protein of interest, in particular as described above;
b) addition of the nucleic acid to an in vitro transcription medium comprising a RNA polymerase, a suitable buffer, a nucleic acid mix, comprising one or more base-modified nucleotides as described above as replacement for one or more of the naturally occurring nucleotides A, G, C and/or U, and optionally one or more naturally occurring nucleotides A, G, C or U if not all the naturally occurring nucleotides A, G, C or U are to be replaced, and optionally a RNase inhibitor;
c) incubation of the nucleic acid in the in vitro transcription medium and in vitro transcription of the nucleic acid;
d) optional purification and removal of the unincorporated nucleotides from the in vitro transcription medium;
e″) transfection of the base-modified nucleic acid obtained in step c) (and optionally d)) into a host cell, and transplantation of the transfected host cell into an organism;
f″) translation of the protein coded for by the base-modified nucleic acid in the organism.

Steps a), b), c) and d) of the in vitro transcription and in vivo translation method according to the invention for increasing the expression of a protein in an organism are identical with steps a), b), c) and d) of the above-described in vitro transcription method according to the invention, of the above-described in vitro transcription and translation method according to the invention for increasing the expression of a protein, and of the above-described in vitro transcription and translation method according to the invention for increasing the expression of a protein in a host cell.

Host cells in step e″) can here also include autologous cells, i.e. cells that are removed from a patient and returned again (cells belonging to the body). Such autologous cells reduce the risk of rejection by the immune system in in vivo applications. In the case of autologous cells, (healthy or diseased) cells from the affected body regions/organs of the patient are preferably used. Transfection methods are preferably those as described above for step e). In step e″), transplantation of the host cell into an organism is carried out in addition to step e). An organism or a living being in connection with the present invention is typically an animal, including cattle, pigs, mice, dogs, cats, rodents, hamsters, rabbits, etc., as well as humans. Alternatively to steps e″) and f″), the isolation and/or purification according to steps f)/f′) and/or g)/g′) and subsequent administration of the translated (therapeutically active) protein to the living being can be carried out. The administration can be carried out as described for pharmaceutical compositions.

In step f″), the translation of the protein coded for by the base-modified nucleic acid is carried out in the organism. The translation takes place by host-cell-specific systems in dependence on the host cell used.

Independently of steps (a) to (d), the nucleic acid used according to the invention can also be expressed by an in vitro translation method of steps (e″) to (g″), which, as such, also forms part of the present invention.

Another embodiment of the present invention refers to cell-based approaches for therapeutic purposes. Accordingly, cells explanted from the body of the organism, in particular humans, are cultured in vitro. These cells are transfected by an base-modified RNA as disclosed herein. The base-modified RNA is provided as described herein elsewhere. In more detail, transfection of the cells or tissues in vitro or in vivo is in general carried out by adding the base-modified RNA provided and/or prepared according to step a) to the cells or tissue. Preferably, the complexed RNA then enters the cells by using cellular mechanisms, e.g. endocytosis. Addition of the complexed RNA to the cells or tissues may occur directly without any further additional components. Alternatively, addition of the base-modified RNA provided and/or prepared according to step a) id added to the cells or tissues may occur as a composition as defined herein, (optionally containing further additional components).

Cells (or host cells) in this context for transfection of the base-modified RNA (provided and/or prepared according to step a)) in vitro includes any cell, and preferably, with out being restricted thereto, cells, which allow expression of a protein encoded by the base-modified RNA. Cells in this context preferably include cultured eukaryotic cells (e.g. yeast cells, plant cells, animal cells and human cells) or prokaryotic cells (e.g. bacteria cells etc.). Cells of multicellular organisms are preferably chosen if posttranslational modifications, e.g. glycosylation of the encoded protein, are necessary (N- and/or O-coupled). In contrast to prokaryotic cells, such (higher) eukaryotic cells render possible posttranslational modification of the protein synthesized. The person skilled in the art knows a large number of such higher eukaryotic cells or cell lines, e.g. 293T (embryonal kidney cell line), HeLa (human cervix carcinoma cells), CHO (cells from the ovaries of the Chinese hamster) and further cell lines, including such cells and cell lines developed for laboratory purposes, such as, for example, hTERT-MSC, HEK293, Sf9 or COS cells. Suitable eukaryotic cells furthermore include cells or cell lines which are impaired by diseases or infections, e.g. cancer cells, in particular cancer cells of any of the types of cancer mentioned here in the description, cells impaired by HIV, and/or cells of the immune system or of the central nervous system (CNS). Suitable cells can likewise be derived from eukaryotic microorganisms, such as yeast, e.g. Saccharomyces cerevisiae (Stinchcomb et al., Nature, 282:39, (1997)), Schizosaccharomyces pombe, Candida, Pichia, and filamentous fungi of the genera Aspergillus, Penicillium, etc. Human cells or animal cells, e.g. of animals as mentioned here, are particularly preferred as eukaryotic cells. Furthermore, antigen presenting cells (APCs) may be used for ex vivo transfection of the bas-modified RNA according to the present invention. Also included herein are dendritic cells, which may be used for ex vivo transfection of the complexed RNA according to the present invention. These APCs, in particular dendritic cells are particularly useful, if the base-modified RNA codes for an antigen of a pathogenic organism or a tumor antigen. Hereby, the retransplanted APCs are able to express the antigen in vivo and to provoke an adequate, adaptive immune response in vivo. Accordingly, the retransplanted, preferably in to the blood, APCs trigger an adequate immune response which allows the organism to immunologically attack the tumor or the pathogenic organism. This method may also allow to treat autoimmune diseases, since the autoantigen presented after transfection on the APCs may desensitize the organism (if an adequate administration protocol is followed) and thereby suppresses the Organism's immune response.

Suitable cells likewise include prokaryotic cells, such as e.g. bacteria cells, e.g. from Escherichia coli or from bacteria of the general Bacillus, Lactococcus, Lactobacillus, Pseudomonas, Streptomyces, Streptococcus, Staphylococcus, preferably E. coli, etc.

In summary, this embodiment allows to pursue a cell-based gene therapeutic approach, whereby (a) base-modified RNA or a composition containing a base-modified RNA is provided, (b) cells are explanted from a multicellular organism (if required), (c) cells are transfected by a base-modified RNA of the invention and (d) cells are retransplanted into the organism. This approach holds, if autologous cells are used. If there is no need to use autologous cells, also allogenic cells may be used (e.g. established cell lines), which are then transfected and re-implanted. Accordingly, the allogenic cells may allow to skip step (b). While the ex vivo method is one embodiment, the invention encompasses also the use of a base-modified RNA for extracellular transfection of cells or tissues as disclosed above.

The present invention also provides a process for the preparation of a RNA library or compositions containing an RNA library, comprising the steps:
(a) preparation/provision of a cDNA library, or a part thereof, from any cell or tissue, in particular a tumour tissue of a patient,
(b) preparation/provision of a matrix for in vitro transcription of a base-modified RNA according to the invention with the aid of the cDNA library or a part thereof and
(c) in vitro transcribing of the matrix.

The any tissue of the patient can be obtained e.g. by a simple biopsy (e.g. a tumoue tissue). However, it can also be provided by surgical removal of e.g. tumour-invaded tissue. The preparation/provision of the cDNA library or a part thereof according to step (a) of the preparation process of the present invention can moreover be carried out after the corresponding tissue has been deep-frozen for storage, preferably at temperatures below −70° C. For preparation of the cDNA library or a part thereof, isolation of the total RNA, e.g. from a tumour tissue biopsy, is first carried out. Processes for this are described e.g. in Maniatis et al., supra. Corresponding kits are furthermore commercially obtainable for this, e.g. from Roche AG (e.g. the product “High Pure RNA Isolation Kit”). The corresponding poly(A⁺) RNA is isolated from the total RNA in accordance with processes known to a person skilled in the art (cf. e.g. Maniatis et al., supra). Appropriate kits are also commercially obtainable for this. An example is the “High Pure RNA Tissue Kit” from Roche AG. Starting from the poly(A⁺) RNA obtained in this way, the cDNA library is then prepared (in this context cf. also e.g. Maniatis et al., supra). For this step in the preparation of the cDNA library also, commercially obtainable kits are available to a person skilled in the art, e.g. the “SMART PCR cDNA Synthesis Kit” from Clontech Inc. The individual sub-steps from the poly(A⁺) RNA to the double-stranded cDNA may be carried out in accordance with the “SMART PCR cDNA Synthesis Kit” from Clontech Inc.

According to step (b) of the above preparation process, starting from the cDNA library (or a part thereof), a matrix is synthesized for the in vitro transcription. According to the invention, this is effected in particular by cloning the cDNA fragments obtained into a suitable RNA production vector, e.g. a plasmid. For in vitro transcription of the matrix prepared in step (b) according to the invention, these are first linearized with a corresponding restriction enzyme, if they are present as circular plasmid (c)DNA. Preferably, the construct cleaved in this way is purified once more, e.g. by appropriate phenol/chloroform and/or chloroform/phenol/isoamyl alcohol mixtures, before the actual in vitro transcription. By this means it is ensured in particular that the DNA matrix is in a protein-free form. The enzymatic synthesis of the RNA is then carried out starting from the purified matrix. This sub-step takes place in an appropriate reaction mixture comprising the linearized, protein-free DNA matrix in a suitable buffer, to which a ribonuclease inhibitor is preferably added, using a mixture of the required ribonucleotide triphosphates (rATP, RCTP, rUTP and RGTP) either in native form or as base-modified nucleotides and a sufficient amount of a RNA polymerase, e.g. T7 polymerase. Accordingly, an RNA library may be prepared which contains exclusively a specific base modified form of rATP, rCTP, rUTP or rGTP. Also any combination of base-modified nucleotides may be obtained, e.g. base-modified adenosine nucleotides and base modified cytidine nucleotides (e.g. 7-Deazaguanosine-TP and Pseudouridin-TP). Alternatively or additionally, the library may also contain only a certain amount of a base-modified nucleotides of one or more types of the 4 types of nucleotides, which may be influenced by the initial ratio of base-modified/unmodified nucleotides added to the transcription reaction medium (e.g. 20% 7-Deazaguanosine-TP and 80% native Guanosin-TP). Still further, there may be also a combination of different base-modified nucleotides of one or more of the 4 nucleotide types existing, the ration again depending on the initial ratio of the modified nucleotides added to the medium (e.g. a combination of 30% 5-Bromo-cytidin-triphosphat and 70% of 5-Methylcytidin-triphosphat). The reaction mixture is present here in RNase-free water. Preferably, a CAP analogue is also added during the actual enzymatic synthesis of the RNA. After an incubation of an appropriately long period, e.g. 2 h, at 37° C., the DNA matrix is degraded by addition of RNase-free DNase, incubation preferably being carried out again at 37° C.

Preferably, the RNA prepared in this way is precipitated by means of ammonium acetate/ethanol and, where appropriate, washed once or several times with RNase-free ethanol. Finally, the RNA purified in this way is dried and, according to a preferred embodiment, is taken up in RNase-free water. The RNA prepared in this way can moreover be subjected to several extractions with phenol/chloroform or phenol/chloroform/isoamyl alcohol.

According to a further preferred embodiment of the preparation process defined above, only a part of a total cDNA library is obtained and converted into corresponding mRNA molecules. According to the invention, a so-called subtraction library can therefore also be used as part of the total cDNA library in order to provide the mRNA molecules according to the invention. A preferred part of the cDNA library of any tissue (e.g. a tumour tissue) codes for specific proteins of particular interest, while other proteins may be less relevant. E.g. it may be advantageous to prepare a subtraction library of tumour-specific antigens, while house-keeping proteins occurring in any cell may be preferred to be subtracted. For certain tumours, the corresponding antigens are known. According to a further preferred embodiment, the part of the cDNA library which codes for the (tumour) specific antigens can first be defined (i.e. before step (a) of the process defined above). This is preferably effected by determining the sequences of the (tumour)-specific antigens by an alignment with a corresponding cDNA library from healthy tissue. Similar methods may be used to establish RNA libraries containing base-modified RNA sequences, if certain antigens derived from pathogens shall be presented by an inventive RNA library. These antigens may be isolated similarly, subtracting the normal proteins of an infected tissue.

The alignment according to the invention comprises in particular a comparison of the expression pattern of the healthy tissue with that of the (tumour) tissue in question. Corresponding expression patterns can be determined at the nucleic acid level e.g. with the aid of suitable hybridization experiments. For this e.g. the corresponding (m)RNA or cDNA libraries of the tissue can in each case be separated in suitable agarose or polyacrylamide gels, transferred to membranes and hybridized with corresponding nucleic acid probes, preferably oligonucleotide probes, which represent the particular genes (northern and southern blots, respectively). A comparison of the corresponding hybridizations thus provides those genes which are expressed either exclusively by the tumour tissue or to a greater extent therein.

According to a further preferred embodiment, the hybridization experiments mentioned are carried out with the aid of a diagnosis by microarrays (one or more microarrays). A corresponding DNA microarray comprises a defined arrangement, in particular in a small or very small space, of nucleic acid, in particular oligonucleotide, probes, each probe representing e.g. in each case a gene, the presence or absence of which is to be investigated in the corresponding (m)RNA or cDNA library. In an appropriate microarrangement, hundreds, thousands and even tens to hundreds of thousands of genes can be represented in this way. For analysis of the expression pattern of the particular tissue, either the poly(A⁺) RNA or, which is preferable, the corresponding cDNA is then marked with a suitable marker, in particular fluorescence markers are used for this purpose, and brought into contact with the microarray under suitable hybridization conditions. If a cDNA species binds to a probe molecule present on the microarray, in particular an oligonucleotide probe molecule, a more or less pronounced fluorescence signal, which can be measured with a suitable detection apparatus, e.g. an appropriately designed fluorescence spectrometer, is accordingly observed. The more the cDNA (or RNA) species is represented in the library, the greater will be the signal, e.g. the fluorescence signal. The corresponding microarray hybridization experiment (or several or many of these) is (are) carried out separately for the tumour tissue and the healthy tissue. The genes expressed exclusively or to an increased extent by the tumour tissue can therefore be concluded from the difference between the signals read from the microarray experiments. Such DNA microarray analyses are described e.g. in Schena (2002), Microarray Analysis, ISBN 0-471-41443-3, John Wiley & Sons, Inc., New York, the disclosure content in this respect of this document being included in its full scope in the present invention.

However, the establishing of (tumour) tissue-specific expression patterns is in no way limited to analyses at the nucleic acid level. Methods known from the prior art which serve for expression analysis at the protein level are of course also familiar to a person skilled in the art. There may be mentioned here in particular techniques of 2D gel electrophoresis and mass spectrometry, whereby these techniques advantageously also can be combined with protein biochips (i.e., microarrays at the protein level, in which e.g. a protein extract from healthy or tumour tissue is brought into contact with antibodies and/or peptides applied to the microarray substrate). With regard to the mass spectroscopy methods, MALDI-TOF (“matrix assisted laser desorption/ionization-time of flight”) methods are to be mentioned in this respect. The techniques mentioned for protein chemistry analysis to obtain the expression pattern of tumour tissue in comparison with healthy tissue are described e.g. in Rehm (2000) Der Experimentator: Proteinbiochemie/Proteomics [The Experimenter: Protein Biochemistry/Proteomics], Spektrum Akademischer Verlag, Heidelberg, 3rd ed., to the disclosure content of which in this respect reference is expressly made expressis verbis in the present invention. With regard to protein microarrays, reference is moreover again made to the statements in this respect in Schena (2002), supra.

Any RNA library (cRNA) containing base-modified nucleotides is encompassed by the present invention. An inventive RNA library may also represent only part of the transcriptom (all transcribed mRNA molecule of a cell/tissue) by subtracting the certain mRNA molecules from the original number of RNA molecules. In particular, any RNA library obtainable according to the above method of the invention is also encompassed by the present invention.

The following Examples and Figures are intended to explain and illustrate the preceding description in greater detail, without being limited thereto.

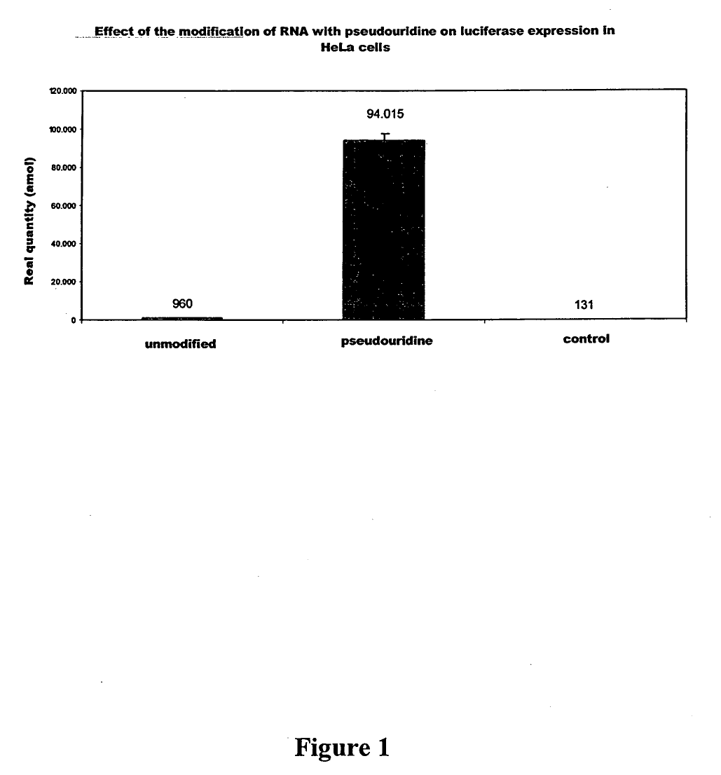

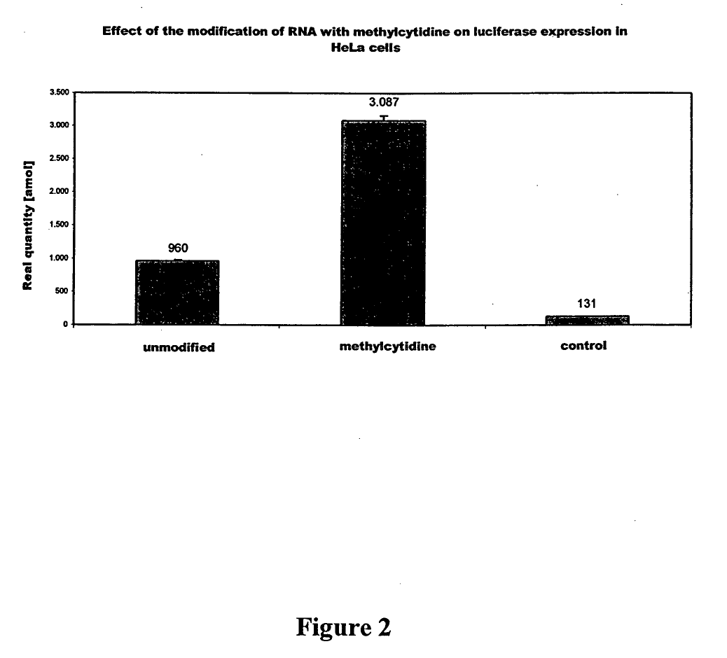

FIG. 1 shows the results of the base modification of luciferase RNA with pseudouridine-5′-triphosphate and subsequent transfection in HeLa cells (see Example 2A). As can be seen in FIG. 2, the overexpression of luciferase was substantially improved (960 amol (attomol) real quantity of the unmodified mRNA sequence compared with 94015 amol real quantity of the base-modified RNA sequence).

FIG. 2 shows the results of the base modifications of luciferase RNA with 5-methylcytidine-5′-triphosphate and subsequent transfection into HeLa cells (see Example 2B). As will be seen in FIG. 2, the overexpression of luciferase was likewise substantially improved (960 amol real quantity of the unmodified mRNA sequence compared with 3087 amol real quantity of the base-modified mRNA sequence).

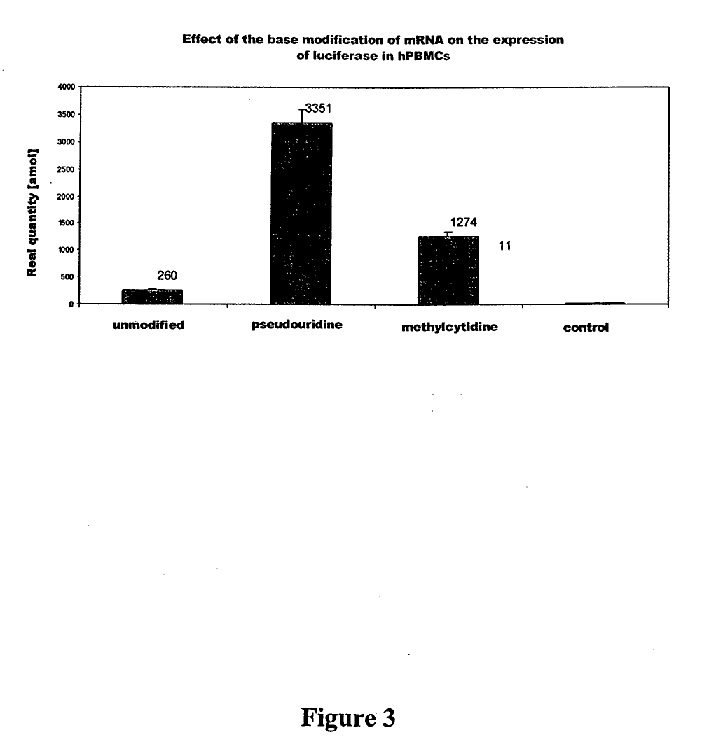

FIG. 3 shows the results of the base modifications of luciferase RNA with pseudouridine-5′-triphosphate and in parallel with 5-methylcytidine-5′-triphosphate and subsequent transfection into hPBMC cells (see Example 3B). As will be seen in FIG. 3, here too the overexpression of luciferase was substantially improved (260 amol real quantity of the unmodified mRNA sequence compared with 3351 amol real quantity of the mRNA sequence modified with pseudouridine-5′-triphosphate and 1274 amol real quantity of the mRNA sequence modified with 5-methylcytidine-5′-triphosphate).

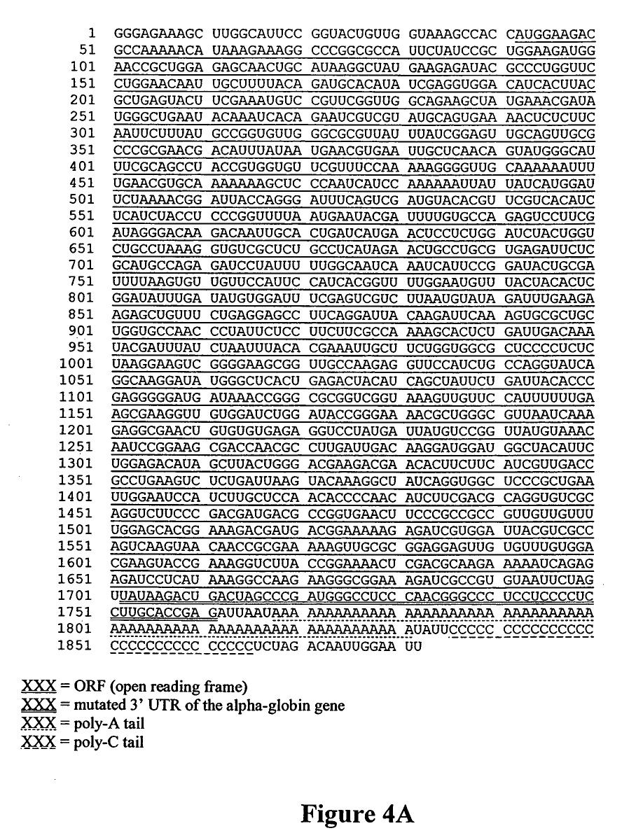

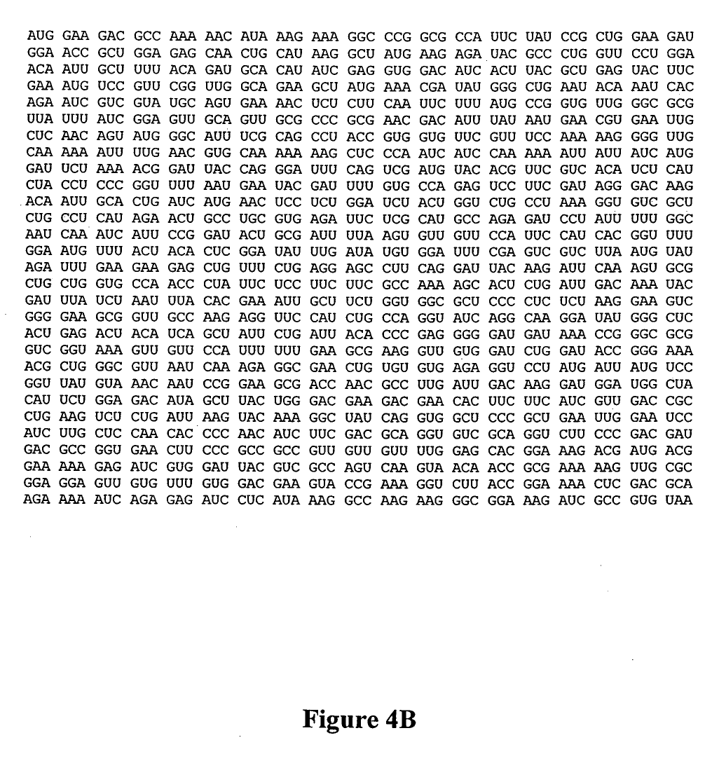

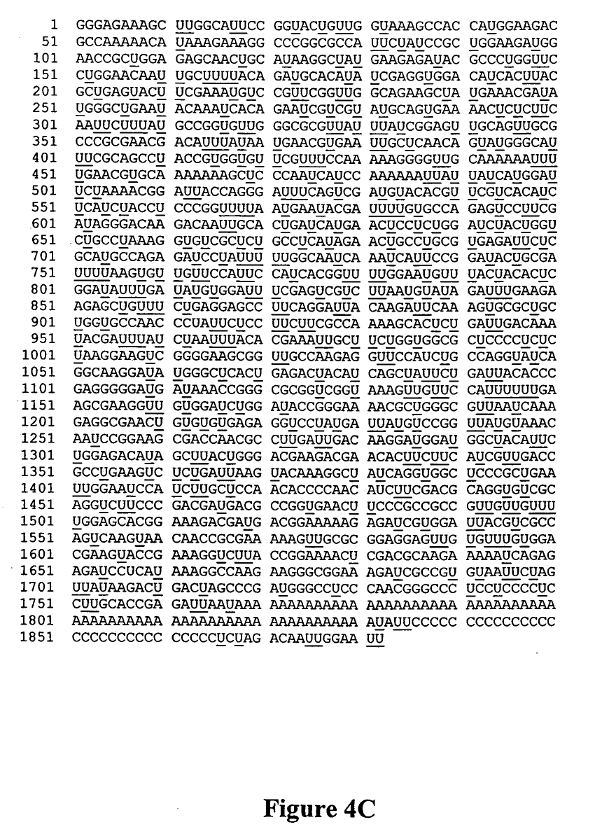

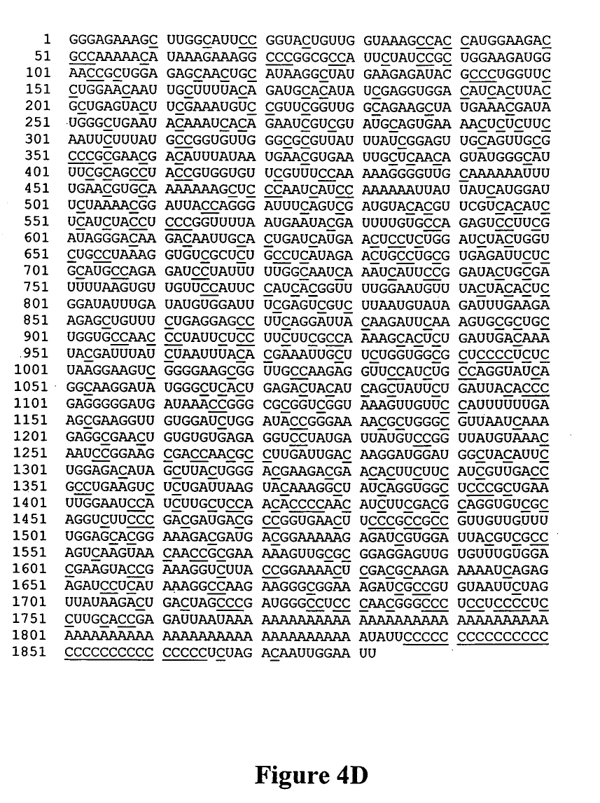

FIG. 4A shows the mRNA sequence of luciferase (SEQ ID NO: 3) with the following further modifications (see Example 1A):
stabilising sequences from alpha-globin gene
poly-A tail of 70 adenosines at the 3′ end
poly-A tail of 30 cytosines at the 3′ end.

FIG. 4B shows the natural coding mRNA sequence of luciferase (SEQ ID NO: 4) (see Example 1A)

FIG. 4C shows the mRNA sequence of luciferase modified with pseudouridine (SEQ ID NO: 5) with the following further modifications (see Example 1B):
stabilising sequences from alpha-globin gene
poly-A tail of 70 adenosines at the 3′ end
poly-A tail of 30 cytosines at the 3′ end

FIG. 4D shows the methylcytidine-modified mRNA sequence of luciferase (SEQ ID NO: 6) with the following further modifications (see Example 1B):
stabilising sequences from alpha-globin gene
poly-A tail of 70 adenosines at the 3′ end
poly-A tail of 30 cytosines at the 3′ end

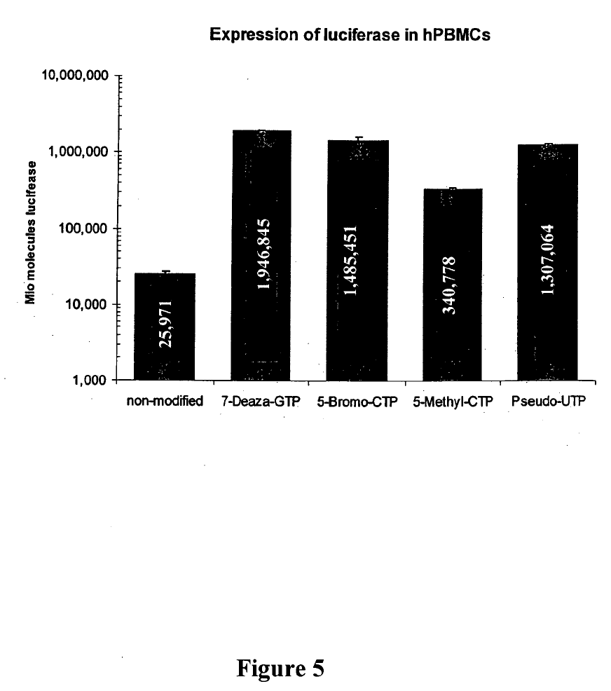

FIG. 5 is a bar graph showing the results of a transfection experiment. hPBMCs were transfected with non-modified or modified mRNA coding for luciferase and luciferase activity was measured 16 h after transfection. The data show that substitution of CTP with 5-Bromo-CTP or 5-Methyl-CTP, substitution of GTP with 7-Deaza-GTP or substitution of UTP with Pseudo-UTP increases the activity of luciferase encoded by modified mRNA compared with luciferase activity in cells which were transfected with non-modified mRNA.

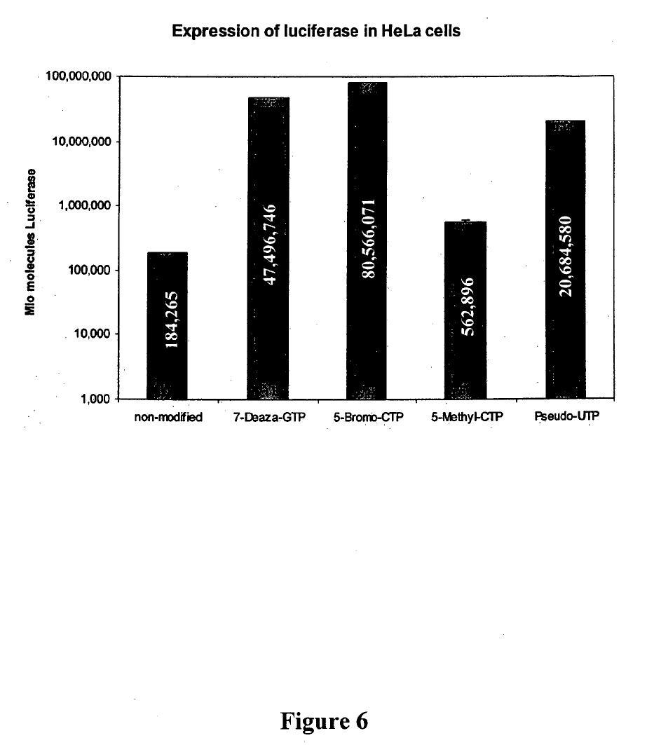

FIG. 6 is a bar graph showing the results of a transfection experiment. HeLa cells were transfected with non-modified or modified mRNA coding for luciferase and luciferase activity was measured 16 h after transfection. The data show that substitution of CTP with 5-Bromo-CTP or 5-Methyl-CTP, substitution of GTP with 7-Deaza-GTP or substitution of UTP with Pseudo-UTP increases the activity of luciferase encoded by modified mRNA compared with luciferase activity in cells which were transfected with non-modified mRNA.

The following Examples illustrate the invention in greater detail, without limiting it.

### EXAMPLE 1

### Base Modifications of RNA

A) mRNA Constructs
A luciferase construct (CAP-Ppluc(wt)-muag-A70-C30) was first produced as template for the base modification (see FIG. 4A, SEQ ID NO: 3), which contained the following modifications in addition to the native coding sequence (SEQ ID NO: 4, see FIG. 4B):
   stabilising sequences from alpha-globin gene
   poly-A tail of about 70 adenosines at the 3′ end
   poly-A tail of 30 cytosines at the 3′ end

### B) In Vitro Transcription

For the introduction of base modifications used according to the invention, the luciferase construct (CAP-Ppluc(wt)-muag-A70-C30, see FIG. 4A, SEQ ID NO: 3) was transcribed by means of T7 polymerase (T7-Opti mRNA Kit, CureVac, Tübingen, Germany). To this end, modified nucleotides were acquired from TriLink (San Diego, USA). All mRNA transcripts contained a poly-A tail about 70 bases long and a 5′-cap structure. The cap structure was obtained by addition of an excess of N7-methylguanosine-5′-triphosphate-5′-guanosine. Pseudouridine-5′-triphosphate-modified mRNA was obtained by adding pseudouridine-5′-triphosphate to the in vitro transcription reaction instead of uridine triphosphate (SEQ ID NO: 5, FIG. 4C) (see below). 5-Methylcytidine-5′-triphosphate-modified RNA was obtained by adding 5-methylcytidine-5′-triphosphate to the in vitro transcription reaction instead of cytidine triphosphate (SEQ ID NO: 6, FIG. 4D) (see below).

### EXAMPLE 2

### Effect of Base Modifications on the Expression of Luciferase in HeLa Cells

A) Modification with pseudouridine-5′-triphosphate
In order to study the effect of various base modifications on the expression of the protein coded for by the mRNA, a plasmid coding for luciferase was subjected to an in vitro transcription using a medium containing pseudouridine-5′-triphosphate instead of uridine-5′-triphosphate. The transcribed mRNA was then transfected into HeLa cells (see above). The expression of luciferase was measured by means of a luminometer after lysis of the cells. The overexpression of luciferase was substantially improved (960 amol real quantity of the unmodified mRNA sequence compared with 94015 amol real quantity of the base-modified mRNA sequence) (see FIG. 1).
   B) Modification with 5-methylcytidine-5′-triphosphate
Alternatively, a plasmid coding for luciferase was subjected to an in vitro transcription using a medium containing 5-methylcytidine-5′-triphosphate instead of cytidine-5′-triphosphate. The transcribed mRNA was then transfected into HeLa cells (see above). The expression of luciferase was measured by means of a luminometer after lysis of the cells. The overexpression of luciferase was substantially improved (960 amol real quantity of the unmodified mRNA sequence compared with 3087 amol real quantity of the base-modified mRNA sequence) (see FIG. 2).

### EXAMPLE 3

### Comparison Tests Relating to the Effect of Base Modifications on the Expression of Luciferase

A) Measurement of Luciferase Expression in HeLa Cells and hPBMCs after Electroporation with Unmodified and Base-Modified mRNA, Coding for Luciferase According to Example 1, HeLa cells and hPBMCs were transfected with 10 μg of unmodified or base-modified RNA by means of the EasyjecT Plus (Peqlab, Erlangen, Germany). 16 hours after the transfection, the cells were lysed with lysis buffer (25 mM Tris-PO₄, 2 mM EDTA, 10% glycerol, 1% Triton-X 100, 2 mM DTT). The supernatants were mixed with luciferin buffer (25 mM glycylglycine, 15 mM MgSO₄, 5 mM ATP, 62.5 μM luciferin) and the luminescence was determined by means of a luminometer (Lumat LB 9507 (Berthold Technologies, Bad Wildbad, Germany)).
B) In a comparison test, a mRNA coding for luciferase and 1) pseudouridine-5′-triphosphate instead of uridine-5′-triphosphate and 2) 5-methylcytidine-5′-triphosphate instead of cytidine-5′-triphosphate were subjected to an in vitro transcription and transfected in hPBMC cells. The expression of luciferase was measured by means of a luminometer after lysis of the cells. Here too, the overexpression of luciferase was substantially improved (260 amol real quantity of the unmodified mRNA sequence compared with 3351 amol real quantity of the mRNA sequence modified with pseudouridine-5′-triphosphate and 1274 amol real quantity of the mRNA sequence modified with 5-methylcytidine-5′-triphosphate) (see FIG. 3).

In summary, luciferase is expressed about 3 times more in HeLa cells and 5 times more in hPBMCs with methylcytidine as base modification of the mRNA in comparison with the unmodified mRNA. The modification of the mRNA with pseudouridine has an even greater effect on the expression of the encoded luciferase. In HeLa cells, for example, luciferase is expressed about 100 times more and in hPBMCs about 13 times more compared with the unmodified mRNA. The effect of the increased overexpression of the protein coded for by a base-modified RNA used according to the invention is accordingly also independent of the chosen host cell.

Corresponding experiments were carried out for comparative purposes luciferase coding base-modified RNA having the base modifications 5-Bromo-CTP (instead of CTP), 5-Methyl-CTP (instead of CTP), 7-Deaza-GTP (instead of GTP) or Pseudo-UTP (instead of UTP). The expression of transfected hPBMCs (FIG. 5) and of transfected HeLa cells (FIG. 6) is shown (Mio molecules luciferase, in logarithmic presentation). Luciferase activity was measured 16 hours after transfection. FIG. 5 shows that the luciferase mRNA was translated in the hPBMCs. Substitution of CTP with 5-Bromo-CTP or 5-Methyl-CTP, substitution of GTP with 7-Deaza-GTP or substitution of UTP with Pseudo-UTP increases the activity of luciferase encoded by modified mRNA considerably (at least 12-fold) compared with luciferase activity in cells which were transfected with non-modified mRNA. The experiments in HeLa cells reflect these findings and show even more clearly the increased expression rate of base-modified RNA according to the invention.

1. Use of a base-modified RNA sequence for increasing the expression of a protein, wherein the base-modified RNA sequence contains at least one base modification and codes for at least one protein.

2. Use according to claim 1, wherein the base-modified RNA is single-stranded or double-stranded, linear or circular, in the form of rRNA, tRNA or mRNA.

3. Use according to claim 1, wherein the base-modified RNA is an mRNA.

4. Use according to claim 1, wherein the base-modified RNA codes for at least one protein selected from the group proteins that are produced by recombinant methods or occur naturally, consisting of growth hormones or growth factors, including TGFα, IGFs (insulin-like growth factors), proteins that influence the metabolism and/or haematopoiesis, including α-anti-trypsin, LDL receptor, erythropoietin (EPO), insulin, GATA-1, or proteins of the blood coagulation system, including factors VIII and XI, etc., [beta]-galactosidase (lacZ), DNA restriction enzymes, including EcoRI, HindIII, lysozymes, or proteases, including papain, bromelain, keratinases, trypsin, chymotrypsin, pepsin, renin (chymosin), suizyme, nortase, or proteins that stimulate the signal transmission of the cell, including cytokines, cytokines of class I of the cytokine family that contain 4 position-specific conserved cysteine residues (CCCC) and a conserved sequence motif Trp-Ser-X-Trp-Ser (WSXWS), including IL-3, IL-5, GM-CSF, the IL-6 sub-family, including IL-6, IL-11, IL-12, or the IL-2 sub-family, including IL-2, IL-4, IL-7, IL-9, IL-15, or the cytokines IL-1α, IL-1β, IL-10, cytokines of class II of the cytokine family (interferon receptor family), which likewise contain 4 position-specific conserved cysteine residues (CCCC) but no conserved sequence motif Trp-Ser-X-Trp-Ser (WSXWS), including IFN-α, IFN-β, IFN-γ, cytokines of the tumour necrosis family, including TNF-α, TNF-β, TNF-RI, TNF-RII, CD40, Fas, or cytokines of the chemokine family, which contain 7 transmembrane helices and interact with G-protein, including IL-8, MIP-1, RANTES, CCR5, CXR4, or apoptosis factors or apoptosis-related or -linked proteins, including AIF, Apaf, for example Apaf-1, Apaf-2, Apaf-3, or APO-2 (L), APO-3 (L), apopain, Bad, Bak, Bax, Bcl-2, Bcl-x_{L}, Bcl-x_{S}, bik, CAD, calpain, caspases, for example caspase-1, caspase-2, caspase-3, caspase-4, caspase-5, caspase-6, caspase-7, caspase-8, caspase-9, caspase-10, caspase-11, ced-3, ced-9, c-Jun, c-Myc, crm A, cytochrome C, CdR1, DcR1, DD, DED, DISC, DNA-PK_{CS}, DR3, DR4, DR5, FADD/MORT-1, FAK, Fas (Fas ligand CD95/fas (receptor)), FLICE/MACH, FLIP, fodrin, fos, G-actin, Gas-2, gelsolin, granzymes A/B, ICAD, ICE, JNK, lamin A/B, MAP, MCL-1, Mdm-2, MEKK-1, MORT-1, NEDD, NF-_{κB, NuMa, p}53, PAK-2, PARP, perforin, PITSLRE, PKCδ, pRb, presenilin, prICE, RAIDD, Ras, RIP, sphingomyelin ase, thymidine kinase from Herpes simplex, TRADD, TRAF2, TRAIL, TRAIL-R1, TRAIL-R2, TRAIL-R3, transglutaminase, or antigens, including tumour-specific surface antigens (TSSAs), including 5T4, α5β1-integrin, 707-AP, AFP, ART-4, B7H4, BAGE, β-catenin/m, Bcr-abl, MN/C IX antigen, CA125, CAMEL, CAP-1, CASP-8, β-catenin/m, CD4, CD19, CD20, CD22, CD25, CDC27/m, CD 30, CD33, CD52, CD56, CD80, CDK4/m, CEA, CT, Cyp-B, DAM, EGFR, ErbB3, ELF2M, EMMPRIN, EpCam, ETV6-AML1, G250, GAGE, GnT-V, Gp100, HAGE, HER-2/new, HLA-A*02011-R170I, HPV-E7, HSP70-2M, HAST-2, hTERT (or hTRT), iCE, IGF-1R, IL-2R, IL-5, KIAA0205, LAGE, LDLR/FUT, MAGE, MART-1/melan-A, MART-2/Ski, MC1R, myosin/m, MUC1, MUM-1, -2, -3, NA88-A, PAP, proteinase-3, p190 minor bcr-abl, Pml/RARα, PRAME, PSA, PSM, PSMA, RAGE, RU1 or RU2, SAGE, SART-1 or SART-3, survivin, TEL/AML1, TGFβ, TPI/m, TRP-1, TRP-2, TRP-2/INT2, VEGE and WT1, or sequences including NY-Eso-1 or NY-Eso-B, or proteins or protein sequences that have a sequence identity of at least 80% with one of the above-described proteins.

5. Use according to claim 1, wherein the base-modified RNA contains at least one base modification selected from the group consisting of 2-amino-6-chloropurineriboside-5′-triphosphate, 2-aminoadenosine-5′-triphosphate, 2thiocytidine-5′-triphosphate, 2-thiouridine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-iodocytidine-5′-triphosphate, 5-iodouridine-5′-triphosphate, 5-methylcytidine-5′-triphosphate, 5-methyluridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, puromycin-5′-triphosphate, and xanthosine-5′-triphosphate.

6. Use according to claim 1, wherein the base modification is selected from the group consisting of 5-methylcytidine-5′-triphosphate, 7-deazaguanosine-5′-triphosphate, 5-bromocytidine-5′-triphosphate and pseudouridine-5′-triphosphate.

7. Use according to claim 1, wherein the base-modified mRNA does not contain any backbone and sugar modifications.

8. Use according to claim 1, wherein the base-modified mRNA contains at least one backbone and/or at least one sugar modification.

9. Use according to claim 1, wherein the base-modified mRNA additionally has a G/C content in the coding region of the base-modified RNA that is greater than the G/C content of the coding region of the native RNA sequence, the amino acid sequence that is coded for being unchanged as compared with the wild type.

10. Use according to claim 1, wherein the coding region of the base-modified RNA is changed as compared with the coding region of the native RNA in such a manner that at least one codon of the native RNA coding for a tRNA that is relatively rare in the cell is replaced by a codon coding for a tRNA that is relatively frequent in the cell and that carries the same amino acid as the relatively rare tRNA.

11. Use according to claim 1, wherein the base-modified RNA additionally contains a 5′-cap structure selected from the group consisting of m7G(5′)ppp(5′(A,G(5′)ppp(5′)A and G(5′)ppp(5′)G.

12. Use according to claim 1, wherein the base-modified RNA additionally contains a poly-A tail of at least 50 nucleotides.

13. Use according to claim 1, wherein the base-modified RNA contains a poly-A tail of at least 20 nucleotides.

14. Use according to claim 1, wherein the base-modified RNA additionally codes for a tag for purification selected from the group consisting of a hexahistidine tag (HIS tag, polyhistidine tag), a streptavidin tag (strep tag), a SBP tag (streptavidin binding tag) or a GST (glutathione S-transferase) tag, or for a tag for purification via an antibody epitope selected from the group consisting of antibody binding tags, a Myc tag, a Swal 1 epitope, a FLAG tag and a HA tag.

15. Use according to claim 1, wherein the base-modified RNA contains a lipid modification.

16. Use of a base-modified RNA sequence as defined in claim 1 for the preparation of a pharmaceutical composition for the treatment of tumours and cancer diseases, heart and circulatory diseases, infectious diseases, autoimmune diseases or monogenetic diseases.

17. Use according to claim 16, wherein the pharmaceutical composition additionally contains an adjuvant selected from the group comprising cationic peptides or polypeptides, including protamine, nucleoline, spermine or spermidine, and cationic polysaccharides, including chitosan, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium oxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyldodecanoyl-amide hydroacetate); CALCITRIOL™ (1α,25-dihydroxy-vitamin D3); calcium phosphate gel; CAPTM (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine); ImmTherT™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-γ; interleukin-1β; interleukin-2; interleukin-7; interleukin-12; ISCOMS™ (“Immune Stimulating Complexes”); ISCOPREP 7.0.3.™; liposomes; LOXORIBINE™ (7-allyl-8-oxoguanosine); LT oral adjuvant (E. coli labile enterotoxin-protoxin); microspheres and microparticles of any composition; MF59™; (squalene-water emulsion); MONTANIDE ISA 51™ (purified incomplete Freund's adjuvant); MONTANIDE ISA 720™ (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))ethylamide, monosodium salt); MURAMETIDE™ (Nac-Mur-L-Ala-D-Gln-OCH₃); MURAPALMITINE™ and D-MURAPALMITINE™ (Nac-Mur-L-Thr-D-isoGIn-sn-glyceroldipalmitoyl); NAGO (neuraminidase-galactose oxidase); nanospheres or nanoparticles of any composition; NISVs (non-ionic surfactant vesicles); PLEURAN™ (β-glucan); PLGA, PGA and PLA (homo- and co-polymers of lactic acid and glycolic acid; micro-/nano-spheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON™ (QS-21); Quil-A (Quil-A saponin); S-28463 (4-amino-otec-dimethyl-2-ethoxymethyl-1H-imidazo[4,5-c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, or a nucleic-acid-based adjuvant selected from CpG and/or RNA oligonucleotides, or Toll-like receptor ligands selected from ligands of TLR1, TLR2, TLR3, TLR4, TLR5, TLR6, TLR7, TLR8, TLR9, TLR10, TLR11, TLR12 or TLR13 or homologues thereof.

18. Use according to claim 16, wherein the pharmaceutical composition is a vaccine.

19. Use according to claim 16, wherein the cancer or tumour diseases are selected from the group consisting of melanomas, malignant melanomas, colon carcinomas, lymphomas, sarcomas, blastomas, renal carcinomas, gastrointestinal tumours, gliomas, prostate tumours, bladder cancer, rectal tumours, stomach cancer, oesophageal cancer, pancreatic cancer, liver cancer, mammary carcinomas (=breast cancer), uterine cancer, cervical cancer, acute myeloid leukaemia (AML), acute lymphoid leukaemia (ALL), chronic myeloid leukaemia (CML), chronic lymphocytic leukaemia (CLL), hepatomas, various virus-induced tumours such as, for example, papilloma virus-induced carcinomas (e.g. cervical carcinoma=cervical cancer), adenocarcinomas, herpes virus-induced tumours (e.g. Burkitt's lymphoma, EBV-induced B-cell lymphoma), heptatitis B-induced tumours (hepatocell carcinomas), HTLV-1- and HTLV-2-induced lymphomas, acoustic neuroma, lung carcinomas (=lung cancer=bronchial carcinoma), small-cell lung carcinomas, pharyngeal cancer, anal carcinoma, glioblastoma, rectal carcinoma, astrocytoma, brain tumours, retinoblastoma, basalioma, brain metastases, medulloblastomas, vaginal cancer, pancreatic cancer, testicular cancer, Hodgkin's syndrome, meningiomas, Schneeberger disease, hypophysis tumour, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumours, oligodendroglioma, vulval cancer, intestinal cancer, colon carcinoma, oesophageal carcinoma (=Oesophageal cancer), wart involvement, tumours of the small intestine, craniopharyngeomas, ovarian carcinoma, genital tumours, ovarian cancer (═Ovarian carcinoma), pancreatic carcinoma (=pancreatic cancer), endometrial carcinoma, liver metastases, penile cancer, tongue cancer, gall bladder cancer, leukaemia, plasmocytoma, lid tumour and prostate cancer (=prostate tumours).

20. Use according to claim 16, wherein the infectious diseases are selected from the group consisting of influenza, malaria, SARS, yellow fever, AIDS, Lyme borreliosis, Leishmaniasis, anthrax, meningitis, viral infectious diseases such as AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio (childhood lameness), pseudo-croup, fifth disease, rabies, warts, West Nile fever, chickenpox, cytomegalic virus (CMV), bacterial infectious diseases such as miscarriage (prostate inflammation), anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis (colpitis), soft chancre, and infectious diseases caused by parasites, protozoa or fungi, such as amoebiasis, bilharziosis, Chagas disease, Echinococcus, fish tapeworm, fish poisoning (Ciguatera), fox tapeworm, athlete's foot, canine tapeworm, candidosis, yeast fungus spots, scabies, cutaneous Leishmaniosis, lambliasis (giardiasis), lice, malaria, microscopy, onchocercosis (river blindness), fungal diseases, bovine tapeworm, schistosomiasis, sleeping sickness, porcine tapeworm, toxoplasmosis, trichomoniasis, trypanosomiasis (sleeping sickness), visceral Leishmaniosis, nappy/diaper dermatitis, or infections caused by miniature tapeworm.

21. Use according to claim 16, wherein the heart and circulatory diseases are selected from the group consisting of coronary heart disease, arteriosclerosis, apoplexia, hypertonia, and neuronal diseases selected from Alzheimer's disease, amyotrophic lateral sclerosis, dystonia, epilepsy, multiple sclerosis and Parkinson's disease.

22. Use according to claim 16, wherein the auto immune diseases are selected from the group consisting of type I autoimmune diseases or type II autoimmune diseases or type III autoimmune diseases or type IV autoimmune diseases, such as, for example, multiple sclerosis (MS), rheumatoid arthritis, diabetes, type I diabetes (Diabetes mellitus), systemic lupus erythematosus (SLE), chronic polyarthritis, Basedow's disease, autoimmune forms of chronic hepatitis, colitis ulcerosa, type I allergy diseases, type II allergy diseases, type III allergy diseases, type IV allergy diseases, fibromyalgia, hair loss, Bechterew's disease, Crohn's disease, Myasthenia gravis, neurodermitis, Polymyalgia rheumatica, progressive systemic sclerosis (PSS), psoriasis, Reiter's syndrome, rheumatic arthritis, psoriasis and vasculitis.

23. Use according to claim 16, wherein the monogenetic diseases are selected from the group consisting of autosomal-recessive inherited diseases, such as, for example, adenosine deaminase deficiency, familial hypercholesterolaemia, Canavan's syndrome, Gaucher's disease, Fanconi anaemia, neuronal ceroid lipofuscinoses, mucoviscidosis (cystic fibrosis), sickle cell anaemia, phenylketonuria, alcaptonuria, albinism, hypothyreosis, galactosaemia, alpha-1-anti-trypsin deficiency, Xeroderma pigmentosum, Ribbing's syndrome, mucopolysaccharidoses, cleft lip, jaw, palate, Laurence Moon Biedl Bardet sydrome, short rib polydactylia syndrome, cretinism, Joubert's syndrome, type II progeria, brachydactylia, adrenogenital syndrome, and X-chromosome inherited diseases, such as, for example, colour blindness, e.g. red/green blindness, fragile X syndrome, muscular dystrophy (Duchenne and Becker-Kiener type), haemophilia A and B, G6PD deficiency, Fabry's disease, mucopolysaccharidosis, Norrie's syndrome, Retinitis pigmentosa, septic granulomatosis, X-SCID, ornithine transcarbamylase deficiency, Lesch-Nyhan syndrome, or from autosomal-dominant inherited diseases, such as, for example, hereditary angiooedema, Marfan syndrome, neurofibromatosis, type I progeria, Osteogenesis imperfecta, Klippel-Trenaurnay syndrome, Sturge-Weber syndrome, Hippel-Lindau syndrome and tuberosis sclerosis.

24. Base-modified RNA sequence according to claim 1.

25. In vitro transcription method for the preparation of base-modified RNA, comprising the following steps:
a) provision of a (desoxy)ribonucleic acid coding for a protein of interest;
b) addition of the nucleic acid to an in vitro transcription medium comprising a RNA polymerase, a buffer, a nucleic acid mix, comprising one or more base-modified nucleotides as defined in claim 5 as replacement for one or more of the naturally occurring nucleotides A, G, C and/or U, and optionally one or more naturally occurring nucleotides A, G, C or U if not all of the naturally occurring nucleotides A, G, C or U are to be replaced, and optionally a RNase inhibitor;
c) incubation of the nucleic acid in the in vitro transcription medium and in vitro transcription of the nucleic acid;
d) optional purification and removal of the unincorporated nucleotides from the in vitro transcription medium.

26. In vitro transcription and translation method for increasing the expression of a protein, comprising the following steps:
a) provision of a (desoxy)ribonucleic acid coding for a protein of interest;
b) addition of the nucleic acid to an in vitro transcription medium comprising a RNA polymerase, a buffer, a nucleic acid mix, comprising one or more base-modified nucleotides as defined in claim 5 as replacement for one or more of the naturally occurring nucleotides A, G, C and/or U, and optionally one or more naturally occurring nucleotides A, G, C or U if not all of the naturally occurring nucleotides A, G, C or U are to be replaced, and optionally a RNase inhibitor;
c) incubation of the nucleic acid in the in vitro transcription medium and in vitro transcription of the nucleic acid;
d) optional purification and removal of the unincorporated nucleotides from the in vitro transcription medium;
e) addition of the base-modified nucleic acid obtained in step c) (and optionally in step d)) to an in vitro translation medium;
f) incubation of the base-modified nucleic acid in the in vitro translation medium and in vitro translation of the protein coded for by the base-modified nucleic acid;
g) optional purification of the protein translated in step f).

27. In vitro transcription and translation method for increasing the expression of a protein in a host cell, comprising the following steps:
a) provision of a (desoxy)ribonucleic acid coding for a protein of interest;
b) addition of the nucleic acid to an in vitro transcription medium comprising a RNA polymerase, a buffer, a nucleic acid mix, comprising one or more base-modified nucleotides as defined in claim 5 as replacement for one or more of the naturally occurring nucleotides A, G, C and/or U, and optionally one or more naturally occurring nucleotides A, G, C or U if not all of the naturally occurring nucleotides A, G, C or U are to be replaced, and optionally a RNase inhibitor;
c) incubation of the nucleic acid in the in vitro transcription medium and in vitro transcription of the nucleic acid;
d) optional purification and removal of the unincorporated nucleotides from the in vitro transcription medium;
e) transfection of the base-modified nucleic acid obtained in step c) (and optionally d)) into a host cell;
f) incubation of the base-modified nucleic acid in the host cell and translation of the protein coded for by the base-modified nucleic acid in the host cell;
g) optional isolation and/or purification of the protein translated in step f′).

28. Ex vivo therapy method comprising:
(a) optionally explantation of the cells or tissues from a patient;
(b) transfection of the cultured cells/tissues or cells/tissues obtained by step (a) by a base-modified RNA according to claim 24;
(e) optionally transplanting the transfected cells of step (b) into the patient.

29. Method according to claim 28, whereby the transfected cells are antigen presenting cells (APCs).

30. An RNA library containing base-modified RNA sequences according to claim 24.

31. An RNA library according to claim 30, whereby the RNA library is a subtraction library representing a part of the cell/tissue transcriptom.

32. An RNA library obtainable from a method, characterized by
(a) preparation/provision of a cDNA library, or a part thereof, from any cell or tissue,
(b) preparation/provision of a matrix for in vitro transcription of a base-modified RNA according to the invention with the aid of the cDNA library or a part thereof and
(c) in vitro transcription of the matrix.


SEQUENCE LISTING (6 sequences)

SEQ ID NO: 1 - RNA, 13 nt, Artificial sequence
  Description of Artificial sequence Kozak-sequence (see description p. 22)
gccgccaccaugg

SEQ ID NO: 2 - RNA, 15 nt, Artificial sequence
  Description of Artificical sequence generic stabilizing sequence (see description p. 23)
nccancccnnucncc

SEQ ID NO: 3 - RNA, 1882 nt, Artificial sequence
  CAP-Ppluc(wt)-muag-A70-C30, coding luciferase (Fig. 4A)
gggagaaagcuuggcauuccgguacuguugguaaagccaccauggaagacgccaaaaacauaaagaaaggcccggcgccauucuauccgcuggaagauggaaccgcuggagagcaacugcauaaggcuaugaagagauacgcccugguuccuggaacaauugcuuuuacagaugcacauaucgagguggacaucacuuacgcugaguacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugggcauuucgcagccuaccgugguguucguuuccaaaaagggguugcaaaaaauuuugaacgugcaaaaaaagcucccaaucauccaaaaaauuauuaucauggauucuaaaacggauuaccagggauuucagucgauguacacguucgucacaucucaucuaccucccgguuuuaaugaauacgauuuugugccagaguccuucgauagggacaagacaauugcacugaucaugaacuccucuggaucuacuggucugccuaaaggugucgcucugccucauagaacugccugcgugagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuucugaggagccuucaggauuacaagauucaaagugcgcugcuggugccaacccuauucuccuucuucgccaaaagcacucugauugacaaauacgauuuaucuaauuuacacgaaauugcuucugguggcgcuccccucucuaaggaagucggggaagcgguugccaagagguuccaucugccagguaucaggcaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucaaagaggcgaacugugugugagagguccuaugauuauguccgguuauguaaacaauccggaagcgaccaacgccuugauugacaaggauggauggcuacauucuggagacauagcuuacugggacgaagacgaacacuucuucaucguugaccgccugaagucucugauuaaguacaaaggcuaucagguggcucccgcugaauuggaauccaucuugcuccaacaccccaacaucuucgacgcaggugucgcaggucuucccgacgaugacgccggugaacuucccgccgccguuguuguuuuggagcacggaaagacgaugacggaaaaagagaucguggauuacgucgccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaagaucgccguguaauucuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaauauuccccccccccccccccccccccccccccccucuagacaauuggaauu

SEQ ID NO: 4 - RNA, 1653 nt, Pyrocoelia rufa
  Luciferase - coding sequence (Fig. 4B)
auggaagacgccaaaaacauaaagaaaggcccggcgccauucuauccgcuggaagauggaaccgcuggagagcaacugcauaaggcuaugaagagauacgcccugguuccuggaacaauugcuuuuacagaugcacauaucgagguggacaucacuuacgcugaguacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugggcauuucgcagccuaccgugguguucguuuccaaaaagggguugcaaaaaauuuugaacgugcaaaaaaagcucccaaucauccaaaaaauuauuaucauggauucuaaaacggauuaccagggauuucagucgauguacacguucgucacaucucaucuaccucccgguuuuaaugaauacgauuuugugccagaguccuucgauagggacaagacaauugcacugaucaugaacuccucuggaucuacuggucugccuaaaggugucgcucugccucauagaacugccugcgugagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuucugaggagccuucaggauuacaagauucaaagugcgcugcuggugccaacccuauucuccuucuucgccaaaagcacucugauugacaaauacgauuuaucuaauuuacacgaaauugcuucugguggcgcuccccucucuaaggaagucggggaagcgguugccaagagguuccaucugccagguaucaggcaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucaaagaggcgaacugugugugagagguccuaugauuauguccgguuauguaaacaauccggaagcgaccaacgccuugauugacaaggauggauggcuacauucuggagacauagcuuacugggacgaagacgaacacuucuucaucguugaccgccugaagucucugauuaaguacaaaggcuaucagguggcucccgcugaauuggaauccaucuugcuccaacaccccaacaucuucgacgcaggugucgcaggucuucccgacgaugacgccggugaacuucccgccgccguuguuguuuuggagcacggaaagacgaugacggaaaaagagaucguggauuacgucgccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaagaucgccguguaa

SEQ ID NO: 5 - RNA, 1882 nt, Artificial sequence; identical to SEQ ID NO: 3
  CAP-Ppluc(wt)-muag-A70-C30, modified with pseudouridine (Fig. 4C)

SEQ ID NO: 6 - RNA, 1882 nt, Artificial; identical to SEQ ID NO: 3
  CAP-Ppluc(wt)-muag-A70-C30, modified with methylcytidine (Fig. 4D)